(19) United States
(12) Patent Application Publication
(10) Pub. No.: US 2025/0075002 A1
(43) Pub. Date: Mar. 6, 2025

(54) MUC1 and CD16A Antibodies and Methods of Use

(71) Applicant: BeiGene Switzerland GmbH, Basel (CH)

(72) Inventors: Hui LI, Beijing (CN); Qiansheng REN, Beijing (CN); Liang QU, Beijing (CN); Ming JIANG, Beijing (CN); Qi LIU, Beijing (CN); Xin CHEN, Beijing (CN); Yun CHEN, Shanghai (CN); Liu XUE, Beijing (CN); Wenjie WANG, Beijing (CN); Jie PAN, Shanghai (CN); Zhuo LI, Shanghai (CN); Xiaoyan TANG, Beijing (CN); Chichi HUANG, Cambridge, MA (US); Ting SHAO, Shanghai (CN)

(73) Assignee: BeiGene Switzerland GmbH, Basel (CH)

(21) Appl. No.: 18/826,629

(22) Filed: Sep. 6, 2024

Related U.S. Application Data
(63) Continuation of application No. PCT/CN24/79593, filed on Mar. 1, 2024.

(30) Foreign Application Priority Data
Mar. 3, 2023 (WO) PCT/CN23/79507
Jul. 17, 2023 (WO) PCT/CN23/107724

(51) Int. Cl. C07K 16/30; A61K 47/18; A61K 47/26; A61K 47/36; A61P 35/00; C07K 16/28

(52) U.S. Cl. CPC C07K 16/3092; A61K 47/183; A61K 47/26; A61K 47/36; A61P 35/00; C07K 16/283; C07K 2317/31; C07K 2317/40; C07K 2317/565; C07K 2317/71; C07K 2317/732; C07K 2317/92

(57) ABSTRACT

The present disclosure provides for multi-specific antibodies and antigen-binding fragments thereof that bind to human MUC1 and CD16A, pharmaceutical compositions comprising said antibodies, and use of the antibodies or the compositions for treating a disease, such as cancer.

### CROSS-REFERENCE TO RELATED APPLICATIONS

This application is a continuation application of PCT Application No. PCT/CN2024/079593, filed on Mar. 1, 2024, entitled “MUC1 and CD16A Antibodies and Methods of Use,” which claims the benefit of priority of PCT Application No. PCT/CN2023/079507, filed Mar. 3, 2023, entitled “MUC1 and CD16A Antibodies and Methods of Use,” and PCT Application No. PCT/CN2023/107724, filed Jul. 17, 2023, entitled “MUC1 and CD16A Antibodies and Methods of Use,” which are hereby incorporated by reference in their entireties.

### SEQUENCE LISTING

The present application is filed with a Sequence Listing in electronic format. The Sequence Listing is provided as a file entitled “01368-0054-00PCT_SL” created on Feb. 27, 2024, which is 179,126 bytes in size. The information in the electronic format of the sequence listing is incorporated herein by reference in its entirety.

### FIELD OF THE DISCLOSURE

Disclosed herein are multi-specific antibodies or antigen-binding fragments thereof that bind to human MUC1 and human CD16A and a composition comprising said antibody.

### BACKGROUND

Mucin 1 (MUC1; also known as CA 15-3, EMA, MCD, PEM, PUM, KL-6, MAM6, MCKD, PEMT, CD227, H23AG, MCKD1, ADMCKD, ADTKD2), belonging to the mucin family, is a heavily glycosylated heterodimeric membrane-tethered protein normally expressed on the apical surface of glandular or luminal epithelial cells of different tissues that lubricate and hydrate epithelial cell surfaces and protect them against pathogens. However, in most human epithelial cancers including pancreatic, breast, ovarian, lung, and colon carcinomas, MUC1 is hypoglycosylated and aberrantly overexpressed. Additionally, MUC1 is expressed on the entire surface of tumor cells due to loss of apicobasal polarity in cancer cells. Given such properties, MUC1 has been considered a promising therapeutic target for human cancers.

The MUC1 heterodimer consists of a longer N-terminal extracellular domain (ECD) (MUC1-N) and a shorter subunit (MUC1-C) which contains a C-terminal cytoplasmic domain with 69 amino acids, a hydrophobic transmembrane domain with 28 amino acids, and a short ECD with 58 amino acids. The two subunits are non-covalently bonded by hydrogen interactions. The MUC1-N is often shed from the surface of cells and released into the circulation. There is increased shed MUC1 level in the serum of patients with various cancers. Previous multiple MUC1-N targeting therapeutics such as AS1402 (huHMFG-1) and BrevaRex (AR-20.5) failed in clinic, which was mostly due to the shed MUC1 sequestering these anti-MUC1 antibodies, thereby hindering their ability to bind to MUC1 on the surface of cancer cells. To overcome this issue, antibodies binding to MUC1-C could be a more promising strategy for efficient targeting of MUC1-expressing cancer cells.

CD16A (also known as FcγRIIIa), is the low-affinity receptor for IgG1 and IgG3, expressed by natural killer (NK) cells, macrophages, and some circulating monocytes. CD16A can induce activation signals by itself and killing of target cells opsonized by antibodies through antibody-dependent cell-mediated cytotoxicity (ADCC).

The ADCC mechanisms contribute to therapeutic efficacy of multiple widely used tumor-targeting monoclonal antibodies (mAbs) such as trastuzumab and rituximab. And such the role of CD16A is supported by the evidence of clinical response to therapeutic antibodies affected by polymorphisms of CD16A. Patients with homozygous high-affinity variant (CD16A-158VN) display a better clinical response than those heterozygous (CD16A-158V/F), or homozygous for low-affinity variant (CD16A-158F/F) in multiple indications.

So far, there are different strategies to potentiate NK cell response to tumor-targeting therapeutics. The most common strategies are genetic manipulation and glycoengineering of the antibody Fc region to enhance their interaction with CD16A. Another strategy is to work on the format of the antibodies. Bispecific killer engagers or tri-specific killer engagers are generated to efficiently redirect innate immune cells such as NK cells toward tumor cells. These formats can better bridge NK cells and tumor cells and allow high-affinity engagement of CD16A, resulting in superior killing activity to antibodies.

The current disclosure provides for targeting MUC1-C and CD16A via multi-specific antibodies that recruit innate immune cells to MUC1-expressing cells and that would be useful in the treatment of MUC1-expressing cancer.

### SUMMARY OF THE DISCLOSURE

The present disclosure is directed to multi-specific anti-MUC1×CD16A antibodies and antigen-binding fragments thereof.

In embodiments, the present disclosure is directed to a multi-specific antibody or antigen-binding fragment thereof that includes a first antigen binding domain that specifically binds to human MUC1 and a second antigen binding domain that specifically binds to human CD16A.

In embodiments, the present disclosure is directed to a multi-specific antibody or antigen-binding fragment, wherein the first antigen binding domain has high selectivity over human CD16B.

In embodiments, the present disclosure is directed to a multi-specific antibody or antigen-binding fragment, wherein the first antigen binding domain that specifically binds to human MUC1 comprises:
(i). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29;
(ii). a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29.
(iii). a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9; or
(iv). a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19.

In embodiments, the present disclosure is directed to a multi-specific antibody or antigen-binding fragment, wherein the first antigen binding domain comprises:
(i). a heavy chain variable region (VH) comprising an amino acid sequence at least 90, 91, 92, 93, 94, 95, 96, 97, 98, or 99% identical to SEQ ID NO: 30, and a light chain variable region (VL) comprising an amino acid sequence at least 90, 91, 92, 93, 94, 95, 96, 97, 98 or 99% identical to SEQ ID NO: 31;
(ii). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98 or 99% identical to SEQ ID NO: 62;
(iii). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98 or 99% identical to SEQ ID NO: 66;
(iv). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98 or 99% identical to SEQ ID NO: 68;
(v). a heavy chain variable region (VH) comprising an amino acid sequence at least 90, 91, 92, 93, 94, 95, 96, 97, 98, or 99% identical to SEQ ID NO: 10, and a light chain variable region (VL) comprising an amino acid sequence at least 90, 91, 92, 93, 94, 95, 96, 97, 98 or 99% identical to SEQ ID NO: 11; or
(vi). a heavy chain variable region (VH) comprising an amino acid sequence at least 90, 91, 92, 93, 94, 95, 96, 97, 98, or 99% identical to SEQ ID NO: 20, and a light chain variable region (VL) comprising an amino acid sequence at least 90, 91, 92, 93, 94, 95, 96, 97, 98 or 99% identical to SEQ ID NO: 21.

In embodiments, the present disclosure is directed to a multi-specific antibody or antigen-binding fragment, wherein one, two, three, four, five, six, seven, eight, nine, or ten amino acids within one or more of SEQ ID NO: 30, 31, 61, 62, 66, 68, 10, 11, 20, and 21 have been inserted, deleted or substituted.

In embodiments, the present disclosure is directed to a multi-specific antibody or antigen-binding fragment, wherein the first antigen binding domain comprises:
(i). a heavy chain variable region (VH) that comprises SEQ ID NO: 30, and a light chain variable region (VL) that comprises SEQ ID NO: 31;
(ii). a heavy chain variable region (VH) that comprises SEQ ID NO: 61, and a light chain variable region (VL) that comprises SEQ ID NO: 62.
(iii). a heavy chain variable region (VH) that comprises SEQ ID NO: 61, and a light chain variable region (VL) that comprises SEQ ID NO: 66;
(iv). a heavy chain variable region (VH) that comprises SEQ ID NO: 61, and a light chain variable region (VL) that comprises SEQ ID NO: 68;
(v). a heavy chain variable region (VH) that comprises SEQ ID NO: 10, and a light chain variable region (VL) that comprises SEQ ID NO: 11; or
(vi). a heavy chain variable region (VH) that comprises SEQ ID NO: 20, and a light chain variable region (VL) that comprises SEQ ID NO: 21.

In embodiments, the present disclosure is directed to a multi-specific antibody or antigen-binding fragment, wherein the second antigen binding domain that specifically binds to human CD16A comprises:
(i). a heavy chain variable region that comprises (a) a HCDR1 of SEQ ID NO: 109, (b) a HCDR2 of SEQ ID NO: 110, and (c) a HCDR3 of SEQ ID NO: 111; or
(ii). a heavy chain variable region that comprises (a) a HCDR1 of SEQ ID NO: 109, (b) a HCDR2 of SEQ ID NO: 114, and (c) a HCDR3 of SEQ ID NO: 111.

In embodiments, the present disclosure is directed to a multi-specific antibody or antigen-binding fragment, wherein the second antigen binding domain comprises:
(i). a heavy chain variable region (VH) comprising an amino acid sequence at least 90, 91, 92, 93, 94, 95, 96, 97, 98 or 99% identical to SEQ ID NO: 112;
(ii). a heavy chain variable region (VH) comprising an amino acid sequence at least 90, 91, 92, 93, 94, 95, 96, 97, 98 or 99% identical to SEQ ID NO: 115;
(iii). a heavy chain variable region (VH) comprising an amino acid sequence at least 90, 91, 92, 93, 94, 95, 96, 97, 98 or 99% identical to SEQ ID NO: 117; or
(iv). a heavy chain variable region (VH) comprising an amino acid sequence at least 90, 91, 92, 93, 94, 95, 96, 97, 98 or 99% identical to SEQ ID NO: 119.

In embodiments, the present disclosure is directed to a multi-specific antibody or antigen-binding fragment, wherein one, two, three, four, five, six, seven, eight, nine, or ten amino acids within one or more of SEQ ID NO: 112, 115, 117, and 119 have been inserted, deleted, or substituted.

The multi-specific antibody or antigen-binding fragment, wherein the second antigen binding domain comprises:
(i). a heavy chain variable region (VH) that comprises SEQ ID NO: 112;
(ii). a heavy chain variable region (VH) that comprises SEQ ID NO: 115;
(iii). a heavy chain variable region (VH) that comprises SEQ ID NO: 117; or
(iv). a heavy chain variable region (VH) that comprises SEQ ID NO: 119.

In embodiments, the present disclosure is directed to a multi-specific antibody or antigen-binding fragment, wherein;
(i). the first antigen binding domain that specifically binds to human MUC1 comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(ii). the first antigen binding domain that specifically binds to human MUC1 comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(iii). the first antigen binding domain that specifically binds to human MUC1 comprises: a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(iv). a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19. and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(v). the first antigen binding domain that specifically binds to human MUC1 comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (c) a LCDR2 of SEQ ID NO: 28, and (f) a LCDR3 of SEQ ID NO: 29. and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111;
(vi). the first antigen binding domain that specifically binds to human MUC1 comprises: a heavy chain variable region that comprises (a) a HCDR1 of SEQ ID NO: 24, (b) a HCDR2 of SEQ ID NO: 25, and (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111;
(vii). the first antigen binding domain that specifically binds to human MUC1 comprises: a heavy chain variable region that comprises (a) a HCDR1 of SEQ ID NO: 4, (b) a HCDR2 of SEQ ID NO: 5, and (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111; or
(viii). a heavy chain variable region that comprises (a) a HCDR1 of SEQ ID NO: 14, (b) a HCDR2 of SEQ ID NO: 15, and (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111.

In embodiments, the present disclosure is directed to a multi-specific antibody or antigen-binding fragment, wherein;
(i). the first antigen binding domain that specifically binds to human MUC1 comprises:
   a) a heavy chain variable region (VH) that comprises SEQ ID NO: 30, and a light chain variable region (VL) that comprises SEQ ID NO: 31;
   b) a heavy chain variable region (VH) that comprises SEQ ID NO: 61, and a light chain variable region (VL) that comprises SEQ ID NO: 62;
   c) a heavy chain variable region (VH) that comprises SEQ ID NO: 61, and a light chain variable region (VL) that comprises SEQ ID NO: 66;
   d) a heavy chain variable region (VH) that comprises SEQ ID NO: 61, and a light chain variable region (VL) that comprises SEQ ID NO: 68;
   e) a heavy chain variable region (VH) that comprises SEQ ID NO: 10, and a light chain variable region (VL) that comprises SEQ ID NO: 11; or
   f) a heavy chain variable region (VH) that comprises SEQ ID NO: 20, and a light chain variable region (VL) that comprises SEQ ID NO: 21;
(ii). and the second antigen binding domain that specifically binds to human CD16A comprises:
   a) a heavy chain variable region that comprises (a) a HCDR1 of SEQ ID NO: 109, (b) a HCDR2 of SEQ ID NO: 110, (c) a HCDR3 of SEQ ID NO: 111; or
   b) a heavy chain variable region that comprises (a) a HCDR1 of SEQ ID NO: 109, (b) a HCDR2 of SEQ ID NO: 114, (c) a HCDR3 of SEQ ID NO: 111.

In embodiments, the present disclosure is directed to a multi-specific antibody or antigen-binding fragment, wherein;
(i). the first antigen binding domain that specifically binds to human MUC1 comprises:
   a) a heavy chain variable region that comprises (a) a HCDR1 of SEQ ID NO: 24, (b) a HCDR2 of SEQ ID NO: 25, (c) a HCDR3 of SEQ ID NO: 26, and a light chain variable region that comprises: (d) a LCDR1 of SEQ ID NO: 27, (e) a LCDR2 of SEQ ID NO: 28, and (f) a LCDR3 of SEQ ID NO: 29;
   b) a heavy chain variable region that comprises (a) a HCDR1 of SEQ ID NO: 24, (b) a HCDR2 of SEQ ID NO: 25, (c) a HCDR3 of SEQ ID NO: 26, and a light chain variable region that comprises: (d) a LCDR1 of SEQ ID NO: 27, (e) a LCDR2 of SEQ ID NO: 65, and (f) a LCDR3 of SEQ ID NO: 29;
   c) a heavy chain variable region that comprises (a) a HCDR1 of SEQ ID NO: 4, (b) a HCDR2 of SEQ ID NO: 5, (c) a HCDR3 of SEQ ID NO: 6, and a light chain variable region that comprises: (d) a LCDR1 of SEQ ID NO: 7, (e) a LCDR2 of SEQ ID NO: 8, and (f) a LCDR3 of SEQ ID NO: 9;
   d) a heavy chain variable region that comprises (a) a HCDR1 of SEQ ID NO: 14, (b) a HCDR2 of SEQ ID NO: 15, (c) a HCDR3 of SEQ ID NO: 16, and a light chain variable region that comprises: (d) a LCDR1 of SEQ ID NO: 17, (e) a LCDR2 of SEQ ID NO: 18, and (f) a LCDR3 of SEQ ID NO: 19;
(ii). and the second antigen binding domain that specifically binds to human CD16A comprises:
   a) a heavy chain variable region (VH) that comprises SEQ ID NO: 112;
   b) a heavy chain variable region (VH) that comprises SEQ ID NO: 115;
   c) a heavy chain variable region (VH) that comprises SEQ ID NO: 117; or
   d) a heavy chain variable region (VH) that comprises SEQ ID NO: 119.

In embodiments, the present disclosure is directed to a multi-specific antibody or antigen-binding fragment, wherein:
(i). the first antigen binding domain that specifically binds to human MUC1 comprises:
   a) a heavy chain variable region (VH) that comprises SEQ ID NO: 30, and a light chain variable region (VL) that comprises SEQ ID NO: 31;
   b) a heavy chain variable region (VH) that comprises SEQ ID NO: 61, and a light chain variable region (VL) that comprises SEQ ID NO: 62;
   c) a heavy chain variable region (VH) that comprises SEQ ID NO: 61, and a light chain variable region (VL) that comprises SEQ ID NO: 66;
   d) a heavy chain variable region (VH) that comprises SEQ ID NO: 61, and a light chain variable region (VL) that comprises SEQ ID NO: 68;
   e) a heavy chain variable region (VH) that comprises SEQ ID NO: 10, and a light chain variable region (VL) that comprises SEQ ID NO: 11; or
   f) a heavy chain variable region (VH) that comprises SEQ ID NO: 20, and a light chain variable region (VL) that comprises SEQ ID NO: 21;
(ii). and the second antigen binding domain that specifically binds to human CD16A comprises:
   a) a heavy chain variable region (VH) that comprises SEQ ID NO: 112;
   b) a heavy chain variable region (VH) that comprises SEQ ID NO: 115;
   c) a heavy chain variable region (VH) that comprises SEQ ID NO: 117; or
   d) a heavy chain variable region (VH) that comprises SEQ ID NO: 119.

In embodiments, the present disclosure is directed to a multi-specific antibody or antigen-binding fragment, which is a monoclonal antibody, a chimeric antibody, a humanized antibody, a human engineered antibody, a single chain antibody (scFv), a Fab fragment, a Fab′ fragment, or a F(ab′)2 fragment.

In embodiments, the present disclosure is directed to a multi-specific antibody or antigen-binding fragment, wherein the multi-specific antibody is a bispecific antibody.

In embodiments, the present disclosure is directed to a multi-specific antibody or antigen-binding fragment, wherein the multi-specific antibody is BG1222P (SEQ ID NO: 143, SEQ ID NO: 145, and SEQ ID NO: 147).

In embodiments, the present disclosure is directed to a multi-specific antibody or antigen-binding fragment, wherein the antibody or antigen-binding fragment thereof has antibody dependent cellular cytotoxicity (ADCC) or complement dependent cytotoxicity (CDC).

In embodiments, the present disclosure is directed to a multi-specific antibody or antigen-binding fragment, wherein the antibody or antigen-binding fragment thereof has reduced glycosylation or no glycosylation or is hypofucosylated.

In embodiments, the present disclosure is directed to a multi-specific antibody or antigen-binding fragment, wherein the antibody or antigen-binding fragment thereof comprises increased bisecting GlcNac structures.

In embodiments, the present disclosure is directed to a multi-specific antibody or antigen-binding fragment, wherein the Fc domain is an IgG1 with reduced effector function.

In embodiments, the present disclosure is directed to a multi-specific antibody or antigen-binding fragment, wherein the Fc domain is an IgG4.

In embodiments, the present disclosure is directed to a multi-specific antibody or antigen-binding fragment thereof, which includes an amino acid sequence at least 90, 91, 92, 93, 94, 95, 96, 97, 98, or 99% identical to the amino acid sequences of CDR, VH, VL, or the full chain as disclosed herein.

In embodiments, the present disclosure is directed to a multi-specific antibody or antigen-binding fragment thereof, wherein one, two, three, four, five, six, seven, eight, nine, or ten amino acids within the amino acid sequences of one or more of CDR, VH, VL, or the full chain, as disclosed herein, have been inserted, deleted, or substituted.

In embodiments, the present disclosure is directed to a pharmaceutical composition including a multi-specific antibody or antigen-binding fragment thereof as disclosed herein, and a pharmaceutically acceptable carrier. The pharmaceutical composition may include histidine/histidine HCl, trehalose dihydrate, and polysorbate 20.

In embodiments, the present disclosure is directed to a method of treating cancer including administering to a patient in need an effective amount of a multi-specific antibody or antigen-binding fragment as disclosed herein. In embodiments, the present disclosure is directed to an isolated nucleic acid that encodes a multi-specific antibody or antigen-binding fragment disclosed herein.

In embodiments, the present disclosure is directed to a vector including the nucleic acid disclosed herein.

In embodiments, the present disclosure is directed to a host cell including the nucleic acid disclosed herein or the vector disclosed herein.

In embodiments, the present disclosure is directed to a process for producing a multi-specific antibody or antigen-binding fragment thereof including cultivating a host cell disclosed herein and recovering the antibody or antigen-binding fragment from the culture.

In embodiments, the multi-specific antibody of the present disclosure is of IgG1, IgG2, IgG3, or IgG4 isotype. In one embodiment, the antibody of the present disclosure comprises an Fc domain of wild-type human IgG1 (also referred as human IgG1wt or huIgG1) or IgG2.

In one embodiment, the multi-specific antibody of the present disclosure binds to MUC1 with a binding affinity (K_{D}) of from 1×10⁻⁶ M to 1×10⁻¹⁰ M. In another embodiment, the antibody of the present disclosure binds to MUC1 with a binding affinity (K_{D}) of about 1×10⁻⁶ M, about 1×10⁻⁷ M, about 1×10⁻⁸ M, about 1×10⁻⁹ M, or about 1×10⁻¹⁰ M.

In embodiments, the anti-human MUC1 multi-specific antibody of the present disclosure shows a cross-species binding activity to cynomolgus MUC1.

In embodiments, antibodies of the present disclosure have strong Fc-mediated effector functions. In some embodiments, the antibodies mediate antibody-dependent cellular cytotoxicity (ADCC) against MUC1 expressing target cells.

### BRIEF DESCRIPTION OF THE DRAWINGS

FIG. 1 is a schematic diagram of MUC1-SEA-mIgG2a (top) and MUC1-SEA-huIgG1 (bottom), in which “N” is the N-terminus and “C” is the C-terminus.

FIGS. 2A-2F show the binding affinity of purified MUC1 antibodies with human and cyno MUC1 overexpression cells by FACS assay with human IgG1 as a negative control. FIGS. 2A and 2B show chimeric anti-MUC1 monoclonal antibody BG138P binding affinity with human and cyno cells that overexpress MUC1. FIGS. 2C and 2D show chimeric BG346P binding affinity with human and cyno cells that overexpress MUC1. FIGS. 2E and 2F show chimeric BG219P binding affinity with human and cyno cells that overexpress MUC.

FIGS. 3A-3C show epitope binning determination by competitive SPR assays, in which purified human MUC1-mFc antigen was flowed over a chip surface and captured by anti-mouse IgG antibody.

FIG. 4A-4F show the effect of soluble MUC1 on MUC1 antibodies binding to MUC1 expressing cells. FIGS. 4A-C show the binding profiles of chBG138P and chBG219P at different concentrations (30, 3, and 0.3 μg/ml, respectively) in the presence of soluble MUC1, with HMFG1 as a positive control, and mlgG and higG1 as negative controls. FIGS. 4D-F show the binding profiles of chBG138P and chBG346P at different concentrations (30, 3, and 0.3 μg/ml, respectively) in the presence of soluble MUC1, with HMFG1 as a positive control, and migG and hlgG1 as negative controls.

FIGS. 5A-5C show that anti-MUC1 monoclonal antibody chBG138P, which targets the MUC1 membrane proximal region, binds to MUC1-positive cancer cell lines. FIGS. 5A-5C, show that chBG138P binds to MUC1-expressing tumor cell lines HCC827 (FIG. 5A), H1975 (FIG. 5B), and T-47D (FIG. 5C) in a dose dependent manner (with human IgG1 as a negative control).

FIG. 6 shows the schema of a FACS gating strategy for a T cell binding assay. The dashed boxes show the proportion of antibody-bound T cells.

FIGS. 7A-7H show that chimeric anti-MUC1 monoclonal antibodies targeting the MUC1 membrane proximal region, chBG138P (FIGS. 7A, 7B, 7E, and 7F), chBG219P (FIGS. 7E and 7F), or chBG346P (FIGS. 7E and 7F) do not bind to activated T cells, while MUC1 membrane distal portion targeting antibody HMFG1 (FIGS. 7C and 7D) and 16A (FIGS. 7G and 7H) can bind to normal T cells.

FIG. 8A-8D depict that chBP138P and humanized MUC1 antibodies, BG138P-hz2 and BG138P-hz4 (FIGS. 8A and 8B), do not bind to normal T cells, while MUC1 membrane distal portion targeting antibody HMFG1 (FIGS. 8C and 8D) binds to normal activated T cells.

FIG. 9 shows that humanized antibodies huBG219P-Bz0, -E39, and -E43 bind comparably to MUC1-overexpressing cell line ZR-75-1 comparably to compared with chimeric antibody chBG219P.

FIG. 10 shows the ELISA analysis results of a representative top clone BG523P compared to LS21.

FIGS. 11A-11C show the FACS analysis of a representative top clone BG523P compared with LS21.

FIGS. 12A-12B: FIG. 12A shows the FACS binding of BG523P and BG524P to the CD16A 158F overexpressing cell line, NK92mi/CD16A 158F; and FIG. 12B shows the FACS binding of BG523P, BG525P, and BG526P to NK92mi/CD16A F158 cells.

FIG. 13 shows the FACS binding signals of BG523P, BG525P, and BG526P at 300 nM to CD16B overexpressing cell lines, NK92mi/CD16B NA1 and NK92mi/CD16B NA2.

FIGS. 14A-14C show FACS-based human IgG competition for NK92mi/CD16A 158F binding for BG523P and its humanized VHHs (BG525P and BG526P) in the presence or absence of 10 mg/mL recombinant CB6 human IgG1. FIG. 14A shows the IgG competition effect on BG523P binding to NK92mi/CD16A 158F; FIG. 14B shows the IgG competition effect on BG525P binding to NK92mi/CD16A 158F; FIG. 14C shows the IgG competition effect on BG526P binding to NK92mi/CD16A 158F.

FIG. 15 is a schematic diagram of MUC1×CD16A multi-specific antibody BG1222P format.

FIG. 16 shows binding affinity of MUC1×CD16A multi-specific antibody BG1222P to MUC1-expressing tumor cell line T47D.

FIG. 17 shows the comparison of MUC1×CD16A multi-specific antibody BG1222P and huBG219P-E39-AF binding to human CD16A F158 or V158 over-expressing NK92mi cells without (FIGS. 17A, C) or with human IgG competition (FIGS. 17B, D).

FIGS. 18A-18H show the comparison of antibody-dependent cellular cytotoxic activity of MUC1×CD16A multi-specific antibody BG1222P and huBG219P-E39-AF mediated by NK92mi/CD16A F158. The activity was characterized in T47D, HCC827, H358, and MDA-MB-453 cells without (FIGS. 18A, B, C, D) or with (FIGS. 18E, F, G, H) human IgG competition.

FIGS. 19A-19H show the comparison of antibody-dependent cellular cytotoxic activity of MUC1×CD16A multi-specific antibody BG1222P and huBG219P-E39-AF mediated by NK92mi/CD16A V158. The activity was characterized in T47D, HCC827, H358, and MDA-MB-453 cells without (FIGS. 19A, B, C, D) or with (FIG. 19E, F, G, H) human IgG competition.

FIG. 20 shows the comparison of cell lysis activity of MUC1×CD16A multi-specific antibody BG1222P and various anti-MUC1 afucosylated antibodies in human whole blood on cancer cell line T47D.

FIGS. 21A-21D show the comparison of cell lysis activity of MUC1×CD16A multi-specific antibody BG1222P and huBG219P-E39-AF in human whole blood on T47D (FIG. 21A), HCC827 (FIG. 21B), H358 (FIG. 21C), and MDA-MB-453 (FIG. 21D) cells.

FIGS. 22A-22B shows the comparison of phagocytotic activity of MUC1×CD16A multi-specific antibody BG1222P and huBG219P-E39-AF mediated by human M2 macrophage on T47D (FIG. 22A) and MDA-MB-453 (FIG. 22B) cells.

FIGS. 23A and 23B show MUC1×CD16A multi-specific antibody BG1222P and daratumumab induced NK fratricide in NK cells.

FIG. 24 shows the pharmacokinetics profile of MUC1×CD16A multi-specific antibody BG1222P in Cynomolgus after i.v infusion of 5 mg/kg and 25 mg/kg BG1222P.

FIGS. 25A-25C show the effect of soluble MUC1 on MUC1×CD16A multi-specific antibody binding to MUC1 expressing cells. briefly, human MUC1 expressing cells were incubated with 30, 3 and 0.3 μg/ml BG1222P in the presence of serially diluted soluble MUC1 (Shanghai Linc-Bio Science Co. LTD), respectively, then after washing and incubation with anti-human IgG secondary Ab, fluorescence was measured by flow cytometry. FIGS. 25A-C show the binding profile of BG1222P at different concentrations (30, 3, and 0.3 μg/ml, respectively) in the presence of soluble MUC1, with HuVH-HMFG1 as a positive control, and mIgG and hIgG1 as negative controls.

### DEFINITIONS

Unless specifically defined below or elsewhere in this document, all other technical and scientific terms used herein have the meaning commonly understood by one of ordinary skill in the art.

As used herein, including in the appended claims, the singular forms of words such as “a,” “an,” and “the” include their corresponding plural references unless the context clearly dictates otherwise.

The term “or” is used to mean, and is used interchangeably with, the term “and/or” unless the context clearly dictates otherwise.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” can mean within one or more than one standard deviation per the practice in the art. “About” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” should be assumed to be within an acceptable error range for that particular value or composition.

The term “MUC1” or “Mucin 1,” also known as CA 15-3, EMA, MCD, PEM, PUM, KL-6, MAM6, MCKD, PEMT, CD227, H23AG, MCKD1, ADMCKD, or ADTKD2, is one member of the mucin family. The amino acid sequence of human MUC1 is listed as SEQ ID NO: 1 and can also be found at accession number P15941.

The term “CD16A” refers to a type I membrane protein with two Ig-like domains that have low affinity for IgG, and is also known as FCGRIIIA and FCGR3A. The amino acid sequence of human CD16A (P08637) can be found with Uniprot P08637 in Uniprot Database.

The terms “administration,” and “administering,” as used herein, when applied to an animal, human, subject, cell, tissue, organ, or biological fluid, means contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell.

The term “subject” or “patient” herein includes any organism, preferably an animal, more preferably a mammal (e.g., rat, mouse, dog, cat, rabbit, primate) and most preferably a human (e.g., a patient comprising, or at risk of having, a disorder described herein).

“Treating” any disease or disorder refers in one aspect to ameliorating the disease or disorder (i.e., slowing or arresting or reducing the development of the disease or at least one of the clinical symptoms thereof). In another aspect, “treat,” “treating,” or “treatment” refers to alleviating or ameliorating at least one physical parameter including those which may not be discernible by the patient. In yet another aspect, “treat,” “treating,” or “treatment” refers to modulating the disease or disorder, either physically (e.g., stabilization of a discernible symptom), physiologically (e.g., stabilization of a physical parameter), or both.

The term “affinity” as used herein refers to the strength of interaction between antibody and antigen. Within the antigen, the variable regions of the antibody interact through non-covalent forces with the antigen at numerous sites. In general, the more interactions, the stronger the affinity.

The term “antibody” as used herein refers to a polypeptide of the immunoglobulin family that can bind a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL or Vκ) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four framework regions (FRs) arranged from amino-terminus to carboxyl-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The positions of the CDRs and framework regions can be determined using various well known definitions in the art, e.g., Kabat, Chothia, AbM and IMGT (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Al-Lazikani et al., J. Mol. Biol., 273:927-748 (1997); Lefranc, M.-P., The Immunologist, 7, 132-136 (1999); Lefranc, M.-P. et al., Dev. Comp. Immunol., 27, 55-77 (2003)).

The term “antibody” includes, but is not limited to, monoclonal antibodies, human antibodies, humanized antibodies, chimeric antibodies, and anti-idiotypic (anti-Id) antibodies. The antibodies can be of any isotype/class (e.g., IgG, IgE, IgM, IgD, IgA, and IgY), or subclass (e.g., IgG1, IgG2, IgG3, IgG4, IgA1, and IgA2).

In some embodiments, the anti-MUC1 antibodies comprise at least one antigen-binding site. In some embodiments, the anti-MUC1 antibodies comprise an antigen-binding fragment from an MUC1 antibody described herein. In some embodiments, the anti-MUC1 antibody is isolated or recombinant.

In some embodiments, the anti-CD16A antibodies comprise at least one antigen-binding site, at least a variable region. In some embodiments, the anti-CD16A antibodies comprise an antigen-binding fragment from an CD16A antibody described herein. In some embodiments, the anti-CD16A antibody is isolated or recombinant.

The term “monoclonal antibody” or “mAb” or “Mab” herein means a population of substantially homogeneous antibodies, i.e., the antibody molecules in the population are identical in amino acid sequence except for possible naturally occurring mutations that can be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies and is not to be construed as requiring production of the antibody by any particular method. Monoclonal antibodies (mAbs) can be obtained by methods known to those skilled in the art. See, for example Kohler et al., Nature 1975 256:495-497; U.S. Pat. No. 4,376,110: Ausubel et al., CURRENT PROTOCOLS IN MOLECULAR BIOLOGY 1992; Harlow et al., ANTIBODIES: A LABORATORY MANUAL, Cold spring Harbor Laboratory 1988; and Colligan et al., CURRENT PROTOCOLS IN IMMUNOLOGY 1993. The antibodies disclosed herein can be of any immunoglobulin class including IgG, IgM, IgD, IgE, IgA, and any subclass thereof such as IgG1, IgG2, IgG3, IgG4. A hybridoma producing a monoclonal antibody can be cultivated in vitro or in vivo. High titers of monoclonal antibodies can be obtained in in vivo production where cells from the individual hybridomas are injected intraperitoneally into mice, such as pristine-primed Balb/c mice to produce ascites fluid containing high concentrations of the desired antibodies. Monoclonal antibodies of isotype IgM or IgG can be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one “light chain” (about 25 kDa) and one “heavy chain” (about 50-70 kDa). The amino-terminal portion of each chain includes a variable region of about 100 to 110 or more amino acids primarily responsible for antigen recognition. The carboxy-terminal portion of the heavy chain can define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively.

Within light and heavy chains, the variable and constant regions are joined by a “J” region of about 12 or more amino acids, with the heavy chain also including a “D” region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same in primary sequence.

Typically, the variable domains of both the heavy and light chains comprise three hypervariable regions, also called “complementarity determining regions (CDRs),” which are located between relatively conserved framework regions (FR). The CDRs are usually aligned by the framework regions, enabling binding to a specific epitope. In general, from N-terminal to C-terminal, both light and heavy chain variable domains comprise FR-1 (or FR1), CDR-1 (or CDR1), FR-2 (FR2), CDR-2 (CDR2), FR-3 (FR3), CDR-3 (CDR3), and FR-4 (FR4). The positions of the CDRs and framework regions can be determined using various well known definitions in the art, e.g., Kabat, Chothia, AbM, and IMGT (see, e.g., Johnson et al., Nucleic Acids Res., 29:205-206 (2001): Chothia and Lesk, J. Mol. Biol., 196:901-917 (1987); Chothia et al., Nature. 342:877-883 (1989); Chothia et al., J. Mol. Biol., 227:799-817 (1992); Al-Lazikani et al., J. Mol. Biol., 273:927-748 (1997) ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme)). Definitions of antigen combining sites are also described in the following: Ruiz et al., Nucleic Acids Res., 28:219-221 (2000); and Lefranc, M. P., Nucleic Acids Res., 29:207-209 (2001); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). For example, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (HCDR1), 51-57 (HCDR2), and 93-102 (HCDR3), and the CDR amino acid residues in the VL are numbered approximately 27-32 (LCDR1), 50-52 (LCDR2), and 89-97 (LCDR3) (numbering according to Kabat). Under IMGT, the CDR regions of an antibody can be determined using the program IMGT/DomainGap Align.

The term “hypervariable region” means the amino acid residues of an antibody that are responsible for antigen-binding. The hypervariable region comprises amino acid residues from a “CDR” (e.g., LCDR1, LCDR2, and LCDR3 in the light chain variable domain and HCDR1, HCDR2, and HCDR3 in the heavy chain variable domain). See, Kabat et al., (1991) Sequences of Proteins of Immunological Interest. 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term “framework” or “FR” residues means those variable domain residues other than the hypervariable region residues defined herein as CDR residues.

Unless otherwise indicated, an “antigen-binding fragment” means antigen-binding fragments of antibodies, i.e. antibody fragments that retain the ability to bind specifically to the antigen bound by the full-length antibody, e.g., fragments that retain one or more CDR regions. Examples of antigen-binding fragments include, but are not limited to, Fab, Fab′, F(ab′)2, and Fv fragments; diabodies; linear antibodies; single-chain antibody molecules, e.g., single chain Fv (ScFv); nanobodies (or VHH antibody); multi-specific antibodies formed from antibody fragments; and bicyclic peptides (Hurov, K. et al, 2021. Journal for ImmunoTherapy of Cancer, 9(11)).

As used herein, an antibody or antigen-binding antibody fragment “specifically binds” to an antigen (e.g., a protein), meaning the antibody exhibits preferential binding to that target as compared to other proteins, but this specificity does not require absolute binding specificity. A “specific” or “selective” binding reaction is determinative of the presence of the antigen in a heterogeneous population of proteins and other biologics, for example, in a biological sample, blood, serum, plasma or tissue sample. Thus, under certain designated immunoassay conditions, the antibodies or antigen-binding fragments thereof specifically bind to a particular antigen at least two times when compared to the background level and do not specifically bind in a significant amount to other antigens present in the sample. In one aspect, under designated immunoassay conditions, the antibody or antigen-binding fragment thereof, specifically bind to a particular antigen at least ten times when compared to the background level of binding and does not specifically bind in a significant amount to other antigens present in the sample.

“Antigen-binding domain” as used herein, comprises at least six CDRs (or three CDRs in terms of single domain antibody) and specifically binds to an epitope. An “antigen-binding domain” of a multi-specific antibody (e.g., a bispecific antibody) comprises a first antigen binding domain that specifically binds to a first epitope and a second antigen binding domain also comprised of at least three CDRs that specifically binds to a second epitope. Multi-specific antibodies can be bispecific, trispecific, tetraspecific, etc., with antigen binding domains directed to each specific epitope. Multi-specific antibodies can be multivalent (e.g., a bispecific tetravalent antibody) that comprises multiple antigen binding domains, for example, 2, 3, 4, or more antigen binding domains that specifically bind to a first epitope and 2, 3, 4, or more antigen binding domains that specifically bind a second epitope.

The term “human antibody” herein means an antibody that comprises only human immunoglobulin protein sequences. A human antibody can contain murine carbohydrate chains if produced in a mouse, in a mouse cell, or in a hybridoma derived from a mouse cell. Similarly, “mouse antibody” or “rat antibody” mean an antibody that comprises only mouse or rat immunoglobulin protein sequences, respectively.

The term “humanized” or “humanized antibody” means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum,” “hu,” “Hu,” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions can be included to increase affinity, increase stability of the humanized antibody, remove a post-translational modification or for other reasons.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable region, or other combinations of sequences or subsequences. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence. In addition, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The term “equilibrium dissociation constant” or “KD” or “M” refers to the dissociation rate constant (kd, time⁻¹) divided by the association rate constant (ka, time⁻¹, M⁻¹). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M. in some aspects, less than about 10⁻¹¹ M, 10₋₁₂ M or 10⁻¹³ M.

The term “cancer” or “tumor” used herein has the broadest meaning as understood in the art and refers to the physiological condition in mammals that is typically characterized by unregulated cell growth. In the context of the present disclosure, the cancer is not limited to a certain type or location.

In the context of the present disclosure, when reference is made to an amino acid sequence, the term “conservative substitution” means substitution of the original amino acid by a new amino acid that does not substantially alter the chemical, physical, and/or functional properties of the antibody or fragment, e.g., its binding affinity to MUC1 or to CD16A. Common conservative substations of amino acids are well known in the art.

The term “knob-into-hole” technology as used herein refers to amino acids that direct the pairing of two polypeptides together either in vitro or in vivo by introducing a spatial protuberance (knob) into one polypeptide and a socket or cavity (hole) into the other polypeptide at an interface in which they interact. For example, knob-into-holes have been introduced in the Fc:Fc binding interfaces, C_{L}:C_{H}I interfaces or VH/VL interfaces of antibodies (see, e.g., US 2011/0287009, US2007/0178552, WO 96/027011, WO 98/050431, and Zhu et al., 1997, Protein Science, 6:781-788). In some embodiments, knob-into-holes ensure the correct pairing of two different heavy chains together during the manufacture of multi-specific antibodies. For example, multi-specific antibodies having knob-into-hole amino acids in their Fc regions can further comprise single variable domains linked to each Fc region, or further comprise different heavy chain variable domains that pair with similar or different light chain variable domains. Knob-into-hole technology can also be used in the VH or VL regions to also ensure correct pairing.

Examples of algorithms that are suitable for determining percent sequence identity and sequence similarity are the BLAST algorithms, which are described in Altschul et al., Nuc. Acids Res. 25:3389-3402, 1977; and Altschul et al., J. Mol. Biol. 215:403-410, 1990. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as values for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of I1, an expectation (E) of 10, M=5, N=−4 and a comparison of both strands. For amino acid sequences, the BLAST program uses as defaults a word length of 3, and expectation (E) of 10, and the BLOSUM62 scoring matrix (see Henikoff and Henikoff, (1989) Proc. Natl. Acad. Sci. USA 89:10915), alignments (B) of 50, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988), which has been incorporated into the ALIGN program (version 2.0), using a PAM120 weight residue table, a gap length penalty of 12, and a gap penalty of 4. In addition, the percent identity between two amino acid sequences can be determined using the Needleman and Wunsch, J. Mol. Biol. 48:444-453, (1970), algorithm which has been incorporated into the GAP program in the GCG software package using either a BLOSUM62 matrix or a PAM250 matrix, and a gap weight of 16, 14, 12, 10, 8, 6, or 4 and a length weight of 1, 2, 3, 4, 5, or 6.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

In some aspects, the present disclosure provides compositions, e.g., pharmaceutically acceptable compositions, which include anti-MUC1×CD16A multi-specific antibodies as described herein, formulated together with at least one pharmaceutically acceptable excipient. As used herein, the term “pharmaceutically acceptable excipient” includes any and all solvents, dispersion media, isotonic and absorption delaying agents, and the like that are physiologically compatible. The excipient can be suitable for intravenous, intramuscular, subcutaneous, parenteral, rectal, spinal or epidermal administration (e.g. by injection or infusion).

The compositions disclosed herein can be in a variety of forms. These include, for example, liquid, semi-solid and solid dosage forms, such as liquid solutions (e.g., injectable and infusion solutions), dispersions or suspensions, liposomes, and suppositories. A suitable form depends on the intended mode of administration and therapeutic application. One suitable mode of administration is parenteral (e.g., intravenous, subcutaneous, intraperitoneal, intramuscular). In some embodiments, the antibody is administered by intravenous infusion or injection. In certain embodiments, the antibody is administered by intramuscular or subcutaneous injection.

The term “therapeutically effective amount” as herein used, refers to the amount of an antibody that, when administered to a subject for treating a disease, or at least one of the clinical symptoms of a disease or disorder, is sufficient to effect such treatment for the disease, disorder, or symptom. The “therapeutically effective amount” can vary with the antibody, the disease, disorder, and/or symptoms of the disease or disorder, severity of the disease, disorder, and/or symptoms of the disease or disorder, the age of the subject to be treated. and/or the weight of the subject to be treated. An appropriate amount in any given instance can be apparent to those skilled in the art or can be determined by routine experiments. In the case of combination therapy, the “therapeutically effective amount” refers to the total amount of the combination components.

The term “combination therapy” refers to the administration of two or more therapeutic agents to treat a therapeutic condition or disorder. Such administration encompasses co-administration of these therapeutic agents in a substantially simultaneous manner. Such administration also encompasses co-administration in multiple or in separate containers or formulations (e.g., capsules, powders, and liquids) for each active ingredient. Powders and/or liquids can be reconstituted or diluted to a desired dose prior to administration. In addition, “combination therapy” encompasses use of each type of therapeutic agent in a sequential manner, either at approximately the same time or at different times. In either case, the treatment regimen will provide beneficial effects of the drug combination in treating the conditions or disorders described herein.

As used herein, the phrase “in combination with” means that an anti-MUC1×CD16A multi-specific antibody is administered to the subject at the same time as, just before, or just after administration of an additional therapeutic agent. In certain embodiments, an anti-MUC1×CD16A multi-specific antibody is administered as a co-formulation with an additional therapeutic agent.

### DETAILED DESCRIPTION

The present disclosure provides for antibodies, antigen-binding fragments, and anti-MUC1×CD16A multi-specific antibodies. Furthermore, the present disclosure provides antibodies that have desirable pharmacokinetic characteristics and other desirable attributes, and thus can be used for reducing the likelihood of or treating cancer. The present disclosure further provides pharmaceutical compositions comprising the antibodies and methods of making and using such pharmaceutical compositions for the prevention and treatment of cancer and associated disorders.

### Anti-MUC1 Antibodies

The present disclosure provides for antibodies or antigen-binding fragments thereof that specifically bind to MUC1. Antibodies or antigen-binding fragments of the present disclosure include, but are not limited to, the antibodies or antigen-binding fragments thereof, generated as described below.

The present disclosure provides antibodies or antigen-binding fragments that specifically bind to MUC1, wherein said antibodies or antibody fragments (e.g., antigen-binding fragments) comprise a VH domain having an amino acid sequence listed in Table 2 and/or 8. The present disclosure also provides antibodies or antigen-binding fragments that specifically bind MUC1, wherein said antibodies or antigen-binding fragments comprise a HCDR having an amino acid sequence of any one of the HCDRs listed in Tables 2 and 8. In one aspect, the present disclosure provides antibodies or antigen-binding fragments that specifically bind to MUC1, wherein said antibodies comprise (or alternatively, consist of) one, two, three, or more HCDRs having an amino acid sequence of any of the HCDRs listed in Tables 2 and 8.

The present disclosure provides for antibodies or antigen-binding fragments that specifically bind to MUC1, wherein said antibodies or antigen-binding fragments comprise a VL domain having an amino acid sequence listed in Table 2 and/or 8. The present disclosure also provides antibodies or antigen-binding fragments that specifically bind to MUC1, wherein said antibodies or antigen-binding fragments comprise a LCDR having an amino acid sequence of any one of the LCDRs listed in Tables 2 and 8. In particular. the disclosure provides for antibodies or antigen-binding fragments that specifically bind to MUC1, said antibodies or antigen-binding fragments comprise (or alternatively, consist of) one, two, three, or more LCDRs having an amino acid sequence of any of the LCDRs listed in Tables 2 and 8.

Other antibodies or antigen-binding fragments thereof of the present disclosure include amino acids that have been changed, yet have at least 60%, 70%, 80%, 90%, 95%, or 99% identity in the CDR regions with the CDR regions disclosed in Tables 2 and 8. In some aspects, it includes amino acid changes wherein no more than 1, 2, 3, 4, or 5 amino acids have been changed in the CDR regions when compared with the CDR regions depicted in the sequences in Table 2 and 8.

Other antibodies of the present disclosure include those in which the amino acids or nucleic acids encoding the amino acids have been changed, yet have at least 60%, 70%, 80%, 90/c, 95%, or 99% identity to the sequences described in Tables 2 and 8. In some aspects, it includes changes in the amino acid sequences wherein no more than 1, 2, 3, 4, or 5 amino acids have been changed in the variable regions when compared with the variable regions depicted in the sequences described in Table 2s and 8, while retaining substantially the same therapeutic activity.

The present disclosure also provides nucleic acid sequences that encode VH, VL, the full length heavy chain, and the full length light chain of the antibodies that specifically bind to MUC1. Such nucleic acid sequences can be optimized for expression in mammalian cells.

The present disclosure provides antibodies and antigen-binding fragments thereof that bind to an epitope of human MUC1. In certain aspects the antibodies and antigen-binding fragments can bind to the same epitope of MUC1.

The present disclosure also provides for antibodies and antigen-binding fragments thereof that bind to the same epitope as do the anti-MUC1 antibodies described in Tables 2 and 8. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to MUC1 demonstrates that the test antibody can compete with that antibody or antigen-binding fragments thereof for binding to MUC1. Such an antibody can, without being bound to any one theory, bind to the same or a related (e.g., a structurally similar or spatially proximal) epitope on MUC1 as the antibody or antigen-binding fragments thereof with which it competes. In a certain aspect, the antibody that binds to the same epitope on MUC1 as the antibodies or antigen-binding fragments thereof of the present disclosure is a human or humanized monoclonal antibody. Such human or humanized monoclonal antibodies can be prepared and isolated as described herein.

### Anti-CD16A Antibodies

The present disclosure provides for antibodies or antigen-binding fragments thereof that specifically bind to CD16A. Antibodies or antigen-binding fragments of the present disclosure include, but are not limited to, the antibodies or antigen-binding fragments thereof, generated as described below.

The present disclosure provides antibodies or antigen-binding fragments that specifically bind to CD16A, wherein said antibodies or antibody fragments (e.g., antigen-binding fragments) comprise a VH domain having an amino acid sequence listed in Table 23. The present disclosure also provides antibodies or antigen-binding fragments that specifically bind CD16A, wherein said antibodies or antigen-binding fragments comprise a HCDR having an amino acid sequence of any one of the HCDRs listed in Table 23. In one aspect, the present disclosure provides antibodies or antigen-binding fragments that specifically bind to CD16A, wherein said antibodies comprise (or alternatively, consist of) one, two, three, or more HCDRs having an amino acid sequence of any of the HCDRs listed in Table 23.

Other antibodies or antigen-binding fragments thereof of the present disclosure include amino acids that have been changed, yet have at least 60%, 70%, 80%, 90%, 95%, or 99% identity in the CDR regions with the CDR regions disclosed in Table 23. In some aspects, it includes amino acid changes wherein no more than 1, 2, 3, 4, or 5 amino acids have been changed in the CDR regions when compared with the CDR regions depicted in the sequences in Table 23.

Other antibodies of the present disclosure include those where the amino acids or nucleic acids encoding the amino acids have been changed, yet have at least 600, 70%, 80%, 90%, 95%, or 99% identity to the sequences described in Table 23. In some aspects, it includes changes in the amino acid sequences wherein no more than 1, 2, 3, 4, or 5 amino acids have been changed in the variable regions when compared with the variable regions depicted in the sequences in Table 23, while retaining substantially the same therapeutic activity.

The present disclosure also provides nucleic acid sequences that encode VH, VL, the full length heavy chain, and the full length light chain of the antibodies that specifically bind to CD16A. Such nucleic acid sequences can be optimized for expression in mammalian cells.

The present disclosure provides antibodies and antigen-binding fragments thereof that bind to an epitope of human CD16A. In certain aspects the antibodies and antigen-binding fragments can bind to the same epitope of CD16A.

The present disclosure also provides for antibodies and antigen-binding fragments thereof that bind to the same epitope as do the anti-CD16A antibodies described in Table 23. Additional antibodies and antigen-binding fragments thereof can therefore be identified based on their ability to cross-compete (e.g., to competitively inhibit the binding of, in a statistically significant manner) with other antibodies in binding assays. The ability of a test antibody to inhibit the binding of antibodies and antigen-binding fragments thereof of the present disclosure to CD16A demonstrates that the test antibody can compete with that antibody or antigen-binding fragments thereof for binding to CD16A. Such an antibody can, without being bound to any one theory, bind to the same or a related (e.g., a structurally similar or spatially proximal) epitope on CD16A as the antibody or antigen-binding fragments thereof with which it competes. In a certain aspect, the antibody that binds to the same epitope on CD16A as the antibodies or antigen-binding fragments thereof of the present disclosure is a human or humanized monoclonal antibody. Such human or humanized monoclonal antibodies can be prepared and isolated as described herein.

### Anti-MUC1×CD16A Multi-Specific Antibodies

In embodiments, the anti-MUC1 and anti-CD16A antibodies disclosed herein may be incorporated into an anti-MUC1×CD16A multi-specific antibody. An antibody is a multi-specific antibody if, for example, it comprises a number of antigen binding domains, wherein at least one antigen binding domain sequence specifically binds MUC1 as a first epitope and a second antigen binding domain sequence specifically binds CD16A as a second epitope. In embodiments, the multi-specific antibody comprises a third, fourth, or fifth antigen binding domain. In embodiments, the multi-specific antibody is a bispecific antibody, a tri-specific antibody, or tetra-specific antibody. In each embodiment, the multi-specific antibody comprises at least one anti-MUC1 antigen binding domain and at least one anti-CD16A antigen binding domain.

In one embodiment, the multi-specific antibody is a bispecific antibody. As used herein, a bispecific antibody specifically binds only two antigens. The bispecific antibody comprises a first antigen binding domain that specifically binds MUC1 and a second antigen binding domain that specifically binds CD16A. Included is a bispecific antibody comprising a heavy chain variable domain and a light chain variable domain that specifically bind MUC1 as a first epitope and a heavy chain variable domain that specifically binds CD16A as a second epitope. In another embodiment, the bispecific antibody comprises an antigen binding fragment that specifically binds MUC1 and an antigen binding fragment that specifically binds CD16A. When the bispecific antibody comprises antigen binding fragments, the antigen-binding fragment can be a Fab, F(ab′)2, Fv, or a single chain Fv (scFv).

Previous experimentation (Coloma and Morrison, Nature Biotech. 15: 159-163 (1997)) described a tetravalent bispecific antibody that was engineered by fusing DNA encoding a single chain anti-dansyl antibody Fv (scFv) after the C terminus (CH3-scFv) or after the hinge (hinge-scFv) of an IgG3 anti-dansyl antibody. The present disclosure provides multivalent antibodies (e.g. tetravalent antibodies) with at least two antigen binding domains, that can be readily produced by recombinant expression of nucleic acid encoding the polypeptide chains of the antibody. The multivalent antibody herein comprises three to eight, but preferably four, antigen binding domains that specifically bind at least two antigens.

### Linkers

The domains and/or regions of the polypeptide chains of the bispecific tetravalent antibodies disclosed herein may be separated by linker regions of various lengths. In some embodiments, the antigen binding domains are separated from each other, a CL, CH1, hinge, CH2, CH3, or the entire Fc region by a linker region. For example, the polypeptide chains may include the sequence VL1-CL-(linker) VH2-CH1, VH-linker-VL. Such linker regions may comprise a random assortment of amino acids or a restricted set of amino acids. Such linker regions can be flexible or rigid (see US2009/0155275).

Multi-specific antibodies have been constructed by genetically fusing two single chain Fv (scFv) or Fab fragments with or without the use of flexible linkers (Mallender et al., J. Biol. Chem. 1994 269:199-206; Mack et al., Proc. Natl. Acad. Sci. USA. 1995 92:7021-5; Zapata et al., Protein Eng. 1995 8.1057-62); via a dimerization device such as leucine zipper (Kostelny et al., J. Immunol. 1992148:1547-53; de Kruifetal J. Biol. Chem. 1996 271:7630-4) and Ig C/CH1 domains (Muller et al., FEBS Lett. 422:259-64); by diabody (Holliger et al., (1993) Proc. Nat. Acad. Sci. USA. 1998 90:6444-8; Zhu et al., Bio/Technology (NY) 1996 14:192-6); Fab-scFv fusion (Schoonjans et al., J. Immunol. 2000 165:7050-7); and mini antibody formats (Pack et al., Biochemistry 1992.31:1579-84; Pack et al., Bio/Technology 1993 11:1271-7).

The bispecific tetravalent antibodies as disclosed herein comprise a linker region of at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, or more amino acid residues between one or more of their antigen binding domains, CL domains, CH1 domains, hinge regions, CH2 domains, CH3 domains, or Fc regions. In some embodiments, the linker region is comprised of the amino acids glycine and serine. The linker may include the sequence GS, GGS, GSG, SGG, GGG, GGGS (SEQ ID NO: 149), SGGG (SEQ ID NO: 150), GGGGS (SEQ ID NO: 151), GGGGSGS (SEQ ID NO: 152), GGGGSGS (SEQ ID NO: 153), GGGGSGGS (SEQ ID NO: 154), GGGGSGGGGS (SEQ ID NO: 155), GGGGSGGGGSGGGGS (SEQ ID NO: 156), AKTfPKLEEGEFSEAR (SEQ ID NO: 157), AKTfPKLEEGEFSEARV (SEQ ID NO: 158), AKTTPKLGG (SEQ ID NO: 159), SAKTTPKLGG (SEQ ID NO: 160), AKTTPKLEEGEFSEARV (SEQ ID NO: 161), SAKTTP (SEQ ID NO: 162), SAKTTPKLGG (SEQ ID NO: 163), RADAAP (SEQ ID NO: 164), RADAAPTVS (SEQ ID NO: 165), RADAAAAGGPGS (SEQ ID NO: 166), RADAAAA(G4S)4 (SEQ ID NO: 167), SAKTTP (SEQ ID NO: 168), SAKTTPKLGG (SEQ ID NO: 169), SAKTTPKLEEGEFSEARV (SEQ ID NO: 170), ADAAP (SEQ ID NO: 171), ADAAPTVSIFPP (SEQ ID NO: 172), TVAAP (SEQ ID NO: 173), TVAAPSVFIFPP (SEQ ID NO: 174), QPKAAP (SEQ ID NO: 175), QPKAAPSVTLFPP (SEQ ID NO: 176), AKTTPP (SEQ ID NO: 177), AKTTPPSVTPLAP (SEQ ID NO: 178), AKTTAP (SEQ ID NO: 179), AKTTAPSVYPLAP (SEQ ID NO: 180), ASTKGP (SEQ ID NO: 181), ASTKGPSVFPLAP (SEQ ID NO: 182), GENKVEYAPALMALS (SEQ ID NO: 183), GPAKELTPLKEAKVS (SEQ ID NO: 184), or GHEAAAVMQVQYPAS (SEQ ID NO: 185), or any combination thereof (see WO2007/024715).

### Dimerization-Specific Amino Acids

In one embodiment, the multivalent antibody comprises at least one dimerization-specific amino acid change. The dimerization-specific amino acid change may result in “knob-into-hole” interactions, and may increase the likelihood of correct assembly of desired multivalent antibodies. The dimerization-specific amino acids may be within the CH1 domain or the CL domain or combinations thereof. Suitable dimerization-specific amino acids used to pair CH1 domains with other CH1 domains (CH1-CH1) and CL domains with other CL domains (CL-CL) may be found at least in the disclosures of WO2014082179, WO2015181805, and WO2017059551. The dimerization-specific amino acids can also be within the Fc domain and can be in combination with dimerization-specific amino acids within the CH1 or CL domains. In one embodiment, the present disclosure provides a bispecific antibody comprising at least one dimerization-specific amino acid pair.

### Further Alteration of the Framework of Fc Region

In aspects, the Fc region is altered by replacing at least one amino acid residue with a different amino acid residue to alter the effector functions of the antibody. For example, one or more amino acids can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in, e.g., U.S. Pat. Nos. 5,624,821 and 5,648,260. both by Winter et al.

In another aspect, one or more amino acid residues can be replaced with one or more different amino acid residues such that the antibody has altered C1q binding and/or reduced or abolished complement dependent cytotoxicity (CDC). This approach is described in, e.g., U.S. Pat. No. 6,194,551 by Idusogie et al.

In yet another aspect. one or more amino acid residues are changed to thereby alter the ability of the antibody to fix complement. This approach is described in, e.g., the publication WO 94/29351 by Bodmer et al. In a specific aspect, one or more amino acids of an antibody or antigen-binding fragment thereof of the present disclosure are replaced by one or more allotypic amino acid residues for the IgG1 subclass and the kappa isotype. Allotypic amino acid residues also include, but are not limited to, the constant region of the heavy chain of the IgG1, IgG2, and IgG3 subclasses as well as the constant region of the light chain of the kappa isotype as described by Jefferis et al., MAbs. 1:332-338 (2009).

In another aspect, the Fc region is modified to increase the ability of the antibody to mediate antibody dependent cellular cytotoxicity (ADCC) and/or to increase the affinity of the antibody for an Fcγ receptor by modifying one or more amino acids. This approach is described in, e.g., the publication WO00/42072 by Presta. Moreover, the binding sites on human IgG1 for FcγRI, FcγRII, FcγRIII, and FcRn have been mapped and variants with improved binding have been described (see Shields et al., J. Biol. Chem. 276:6591-6604, 2001).

In still another aspect, the glycosylation of the multi-specific antibody is modified. For example, an aglycosylated antibody can be made (i.e., the antibody lacks or has reduced glycosylation). Glycosylation can be altered to, for example, increase the affinity of the antibody for “antigen.” Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation can increase the affinity of the antibody for an antigen. Such an approach is described in, e.g., U.S. Pat. Nos. 5,714,350 and 6,350,861 by Co et al.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with an altered glycosylation pathway. Cells with altered glycosylation pathways have been described in the art and can be used as host cells in which to express recombinant antibodies to thereby produce an antibody with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. Publication WO 03/035835 by Presta describes a variant CHO cell line, Lecl3 cells, with reduced ability to attach fucose to Asn (297)-linked carbohydrates, also resulting in hypofucosylation of antibodies expressed in that host cell (see also Shields et al., (2002) J. Biol. Chem. 277:26733-26740). WO99/54342 by Umana et al. describes cell lines engineered to express glycoprotein-modifying glycosyl transferases (e.g., beta(1,4)-N acetylglucosaminyltransferase III (GnTIII)) such that antibodies expressed in the engineered cell lines exhibit increased bisecting GlcNac structures, which results in increased ADCC activity of the antibodies (see also Umana et al., Nat. Biotech. 17:176-180, 1999).

In another aspect, if a reduction of ADCC is desired, human antibody subclass IgG4 was shown in many previous reports to have only modest ADCC and almost no CDC effector function (Moore G L, et al., 2010 MAbs, 2:181-189). However, natural IgG4 was found less stable in stress conditions such as in acidic buffer or under increasing temperature (Angal, S. 1993 Mol Immunol, 30:105-108; Dall'Acqua, W. et al., 1998 Biochemistry, 37:9266-9273; Aalberse et al., 2002 Immunol, 105:9-19). Reduced ADCC can be achieved by operably linking the antibody to an IgG4 Fc engineered with combinations of alterations that reduce FcγR binding or C1q binding activities, thereby reducing or eliminating ADCC and CDC effector functions. Considering the physicochemical properties of an antibody as a biological drug, one of the less desirable, intrinsic properties of IgG4 is dynamic separation of its two heavy chains in solution to form half antibody, which lead to bi-specific antibodies generated in vivo via a process called “Fab arm exchange” (Van der Neut Kolfschoten M, et al., 2007 Science, 317:1554-157). The mutation of serine to proline at position 228 (EU numbering system) appeared inhibitory to the IgG4 heavy chain separation (Angal, S. 1993 Mol Immunol, 30:105-108; Aalberse et al., 2002 Immunol, 105:9-19). Some of the amino acid residues in the hinge and γFc region were reported to have impact on antibody interaction with Fcγ receptors (Chappel S M, et al., 1991 Proc. Natl. Acad. Sci. USA, 88:9036-9040: Mukherjee, J. et al., 1995 FASEB J, 9:115-119; Armour, K. L. et al., 1999 Eur J Immunol, 29:2613-2624: Clynes, R. A. et al., 2000 Nature Medicine. 6:443-446; Arnold J. N., 2007 Annu Rev immunol, 25:21-50). Furthermore, some rarely occurring IgG4 isoforms in human population can also elicit different physicochemical properties (Brusco, A. et al., 1998 Eur J Immunogenet, 25:349-55; Aalberse et al., 2002 Immunol, 105:9-19). To generate multi-specific antibodies with low ADCC and CDC but with good stability, it is possible to modify the hinge and Fc region of human IgG4 and introduce a number of alterations. These modified IgG4 Fc molecules can be found in SEQ ID NOs: 83-88, U.S. Pat. No. 8,735,553 to Li et al.

### Antibody Production

Antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The present disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 12, SEQ ID NO: 22, SEQ ID NO: 32, and SEQ ID NO: 63. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NO: 13, SEQ ID NO: 23, SEQ ID NO: 33, and SEQ ID NO: 64.

The polynucleotides of the present disclosure can encode the variable region sequence of an anti-MUC1×CD16A antibody. They can also encode both a variable region and a constant region of the antibody. Some of the sequences encode a polypeptide that comprises variable regions of both the heavy chain and the light chain of the exemplified anti-MUC1×CD16A antibodies.

Also provided in the present disclosure are expression vectors and host cells for producing the anti-MUC1×CD16A antibodies. The choice of expression vector depends on the intended host cells in which the vector is to be expressed. The expression vectors may contain a promoter and other regulatory sequences (e.g., enhancers) that are operably linked to the polynucleotides encoding an anti-MUC1×CD16A antibody chain or antigen-binding fragment. In some aspects, an inducible promoter is employed to prevent expression of inserted sequences except under the control of inducing conditions. Inducible promoters include, e.g., arabinose, lacZ, metallothionein promoter, or a heat shock promoter. Cultures of transformed organisms can be expanded under non-inducing conditions without biasing the population for coding sequences whose expression products are better tolerated by the host cells. In addition to promoters, other regulatory elements can also be included for efficient expression of an anti-MUC1×CD16A antibody or antigen-binding fragment thereof. These elements may include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the anti-MUC1×CD16A antibody vectors can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters may be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-MUCxCD16A antibodies. Insect cells in combination with baculovirus vectors can also be used.

In other aspects, mammalian host cells are used to express and produce the anti-MUC1×CD16A antibodies of the present disclosure. Examples include a hybridoma cell line expressing endogenous immunoglobulin genes or a mammalian cell line harboring an exogenous expression vector. These include any normal mortal or normal or abnormal immortal animal or human cells. For example, several suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### Production of Bispecific Antibodies

The presently disclosed bispecific antibodies may be produced using a knob-into-hole (KiH) design, which introduces mutations at the core CH3 domain interface. The resulting heterodimers have a reduced CH3 melting temperature (69° C. or less). On the contrary, the ZW heterodimeric Fc design has a thermal stability of 81.5° C., which is comparable to the wild-type CH3 domain. Specific methods of producing of the presently disclosed antibodies are described in the Examples.

### Methods of Detection and Diagnosis

The antibodies or antigen-binding fragments of the present disclosure are useful in a variety of applications including, but not limited to, methods for the detection of MUC1. In one aspect, the antibodies or antigen-binding fragments are useful for detecting the presence of MUC1 in a biological sample. The term “detecting” as used herein includes quantitative or qualitative detection. In certain aspects, a biological sample comprises a cell or tissue. In other aspects, such tissues include normal and/or cancerous tissues that express MUC1 at higher levels relative to other tissues.

In one aspect, the present disclosure provides a method of detecting the presence of MUC1 in a biological sample. In certain aspects, the method comprises contacting the biological sample with an anti-MUC1×CD16A antibody under conditions permissive for binding of the antibody to an antigen and detecting whether a complex is formed between the antibody and the antigen. The biological sample can include, without limitation, urine, tissue, sputum, or blood.

Also included is a method of diagnosing a disorder associated with expression of MUC1. In certain aspects, the method comprises contacting a test cell with an anti-MUC1×CD16A antibody; determining the level of expression (either quantitatively or qualitatively) of MUC1 expressed by the test cell by detecting binding of the anti-MUC1×CD16A antibody to the MUC1 polypeptide; and comparing the level of expression by the test cell with the level of MUC1 expression in a control cell (e.g., a normal cell of the same tissue origin as the test cell or a non-MUC1 expressing cell), wherein a higher level of MUC1 expression in the test cell as compared to the control cell indicates the presence of a disorder associated with expression of MUC1.

### Pharmaceutical Compositions and Formulations

Also provided are compositions, including pharmaceutical formulations, comprising an anti-MUC1×CD16A antibody or antigen-binding fragment thereof, or polynucleotides comprising sequences encoding an anti-MUC1×CD16A antibody or antigen-binding fragment. These compositions can further comprise suitable carriers, such as pharmaceutically acceptable excipients including buffers, which are well known in the art.

Pharmaceutical formulations of an anti-MUC1×CD16A antibody or antigen-binding fragment as described herein are prepared by mixing such antibody or antigen-binding fragment having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to, buffers such as phosphate, citrate, and other organic acids; antioxidants including ascorbic acid and methionine: preservatives (such as octadecyldimethylbcnzyl ammonium chloride; hexamethonium chloride; benzalkonium chloride: benzethonium chloride; phenol, butyl or benzyl alcohol; alkyl parabens such as methyl or propyl paraben: catechol; resorcinol; cyclohexanol; 3-pentanol; and m-cresol): low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Pat. No. 7,871,607 and 2006/0104968. In one aspect, a sHASEGP is combined with one or more additional glycosaminoglycanases such as chondroitinases.

In embodiments, the formulation includes of L-histidine/L-histidine hydrochloride monohydrate, trehalose, and polysorbate 20. In embodiments, the anti-MUC1×CD16A antibody formulation, after constitution with sterile water for injection, is an isotonic solution including 10 mg/mL anti-MUC1×CD16A antibody, 20 mM histidine/histidine HCl, 240 mM trehalose dihydrate, and 0.02% polysorbate 20, at a pH of approximately 5.5.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

Sustained-release preparations can be prepared. Suitable examples of sustained-release preparations include semipermeable matrices of solid hydrophobic polymers containing the antibody, which matrices are in the form of shaped articles. e.g. films, or microcapsules.

The formulations to be used for in vivo administration are generally sterile. Sterility can be readily accomplished, e.g., by filtration through sterile filtration membranes.

### Equivalent

It is to be understood that, while the anti-human 41g-B7H3 antibodies and antigen binding fragments thereof have been described in conjunction with a detailed description thereof, the foregoing description is intended to illustrate and not limit the scope of the invention, which is defined by the scope of the appended claims. Other aspects, advantages, and modifications are within the scope of the claims below.

It is to be understood that one, some, any, or all of the features of the various embodiments disclosed herein may be combined to form further embodiments of the present disclosure. These and other aspects of the present disclosure will be apparent to those skilled in the art.

### EXAMPLES

### Example 1. Generation of Anti-MUC1 Monoclonal Antibodies Targeting MUC1 Membrane-Proximal Region

### MUC1 Recombinant Protein for Immunization and Binding Assays

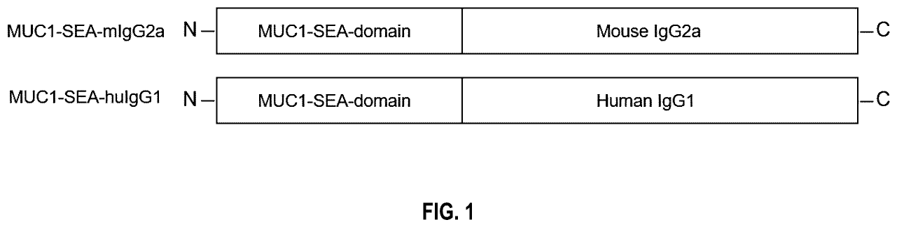

The cDNA coding for the full-length human MUC1 (SEQ ID NO: 1) was synthesized by and purchased from Genewiz (Suzhou, China) based on its Uniprot sequence (UniProtKB: P15941). The coding region of the SEA domain (Sea-urchin Sperm Protein, Enterokinase and Agrin) (SEQ ID NO: 2) of the full-length human MUC1, which consisted of amino acids (AA) 1036-1155 of the full-length MUC1, was PCR-amplified and cloned into pcDNA3.4-based expression vector (Invitrogen, Carlsbad, CA, USA) with the C-terminus fused either to the Fc domain of mouse IgG2a or to the Fc domain of human IgG1 heavy chain, which resulted in two recombinant fusion protein expression plasmids, MUC1-SEA-mIgG2a and MUC1-SEA-huIgG1, respectively. A schematic depiction of MUC1 fusion proteins is shown in FIG. 1. For the recombinant fusion protein production, MUC1-SEA-mIgG2a and MUC1-SEA-huIgG1 plasmids were transiently transfected into Expi293 cells (Thermo Fisher, Waltham, MA, USA) and cultured for 6 days in a CO₂ incubator equipped with a rotating shaker. The supernatant containing the recombinant proteins was collected and cleared by centrifugation. MUC1-SEA-mIgG2a and MUC1-SEA-huIgG1 were purified using a Protein A column (Cat.: 17549852, Cytiva Life Sciences) followed by HiLoad 16/600 Superdex 200 μg size exclusion column (Cat.: 28989335, Cytiva Life Sciences). Both MUC1-SEA-mIgG2a and MUC1-SEA-huIgG1 proteins were dialyzed against phosphate buffered saline (PBS) and stored in a −80° C. freezer in small aliquots.

TABLE 1
Sequences of MUC1

| | SEQ ID | SEQUENCE |
|---|---|---|
| Human | SEQ ID NO: 1 | MTPGTQSPFFLLLLLTVLTVVTGSGHASSTPGGEKETSATQ |
| MUC1 | | RSSVPSSTEKNAVSMTSSVLSSHSPGSGSSTTQGQDVTLAPA |
| (full length) | | TEPASGSAATWGQDVTSVPVTRPALGSTTPPAHDVTSAPDN |
| | | KPAPGSTAPPAHGVTSAPDTRPAPGSTAPPAHGVTSAPDTRP |
| | | APGSTAPPAHGVTSAPDTRPAPGSTAPPAHGVTSAPDTRPAP |
| | | GSTAPPAHGVTSAPDTRPAPGSTAPPAHGVTSAPDTRPAPGS |
| | | TAPPAHGVTSAPDTRPAPGSTAPPAHGVTSAPDTRPAPGSTA |
| | | PPAHGVTSAPDTRPAPGSTAPPAHGVTSAPDTRPAPGSTAPP |
| | | AHGVTSAPDTRPAPGSTAPPAHGVTSAPDTRPAPGSTAPPA |
| | | HGVTSAPDTRPAPGSTAPPAHGVTSAPDTRPAPGSTAPPAH |
| | | GVTSAPDTRPAPGSTAPPAHGVTSAPDTRPAPGSTAPPAHG |
| | | VTSAPDTRPAPGSTAPPAHGVTSAPDTRPAPGSTAPPAHGVT |
| | | SAPDTRPAPGSTAPPAHGVTSAPDTRPAPGSTAPPAHGVTSA |
| | | PDTRPAPGSTAPPAHGVTSAPDTRPAPGSTAPPAHGVTSAPD |
| | | TRPAPGSTAPPAHGVTSAPDTRPAPGSTAPPAHGVTSAPDTR |
| | | PAPGSTAPPAHGVTSAPDTRPAPGSTAPPAHGVTSAPDTRPA |
| | | PGSTAPPAHGVTSAPDTRPAPGSTAPPAHGVTSAPDTRPAPG |
| | | STAPPAHGVTSAPDTRPAPGSTAPPAHGVTSAPDTRPAPGST |
| | | APPAHGVTSAPDTRPAPGSTAPPAHGVTSAPDTRPAPGSTAP |
| | | PAHGVTSAPDTRPAPGSTAPPAHGVTSAPDTRPAPGSTAPPA |
| | | HGVTSAPDTRPAPGSTAPPAHGVTSAPDTRPAPGSTAPPAH |
| | | GVTSAPDTRPAPGSTAPPAHGVTSAPDTRPAPGSTAPPAHG |
| | | VTSAPDTRPAPGSTAPPAHGVTSAPDNRPALGSTAPPVHNV |
| | | TSASGSASGSASTLVHNGTSARATTTPASKSTPFSIPSHHSDT |
| | | PTTLASHSTKTDASSTHHSSVPPLTSSNHSTSPQLSTGVSFFF |
| | | LSFHISNLQFNSSLEDPSTDYYQELQRDISEMFLQIYKQGGF |
| | | LGLSNIKFRPGSVVVQLTLAFREGTINVHDVETQFNQYKTE |
| | | AASRYNLTISDVSVSDVPFPFSAQSGAGVPGWGIALLVLVC |
| | | VLVALAIVYLIALAVCQCRRKNYGOLDIFPARDTYHPMSEY |
| | | PTYHTHGRYVPPSSTDRSPYEKVSAGNGGSSLSYTNPAVAA |
| | | TSANL |
| Human | SEQ ID NO: 2 | LSTGVSFFFLSFHISNLQFNSSLEDPSTDYYQELQRDISEMFL |
| MUC1 | | QIYKQGGFLGLSNIKFRPGSVVVQLTLAFREGTINVHDVET |
| SEA | | QFNQYKTEAASRYNLTISDVSVSDVPFPFSAQSGAG |
| domain | | |
| Cyno | SEQ ID NO: 3 | MTPGTQSPFFLLLILTVLTAATVPEPTTVVTGSGHTNSTPGG |
| MUC1 (full | | EKETSATQRSSMPISTKNAVSMTSRLSSHSPVSGSSTTQGQD |
| length) | | VTLALAMESATGSATTLGHVVTSAPDTSAAPGSTGPPAHV |
| | | VTSAPDTSAAPGSTGPPAHVVTSAPDTVTSASDSASGSAST |
| | | LVHSTTSARATTTPASKSTPFSIPSHHSDTPTTLASHSTKTDA |
| | | SSTHHSTVPPFTSSNHSTSPQLSLGVSFFFLSFHISNLQFNSS |
| | | LEDPSTNYYQQLQRDISELFLQIYKQGDFLGLSNIMFRPGS |
| | | VVVQSTLVFREGTTNVHDVETQFNQRKTEAASRYNLTISDI |
| | | SVRDVPFPFSAQTGAGVPGWGIALLVLVCVLVVMAIVYFIA |
| | | LAVCQCRQKNYRQLDIFPARDAYHPMSEYPTYHTHGRYAP |
| | | AGGTNRSPYEEVSAGNGGSSLSYTNPAVAATSANL |

Cell Lines Stably Expressing Human or Cyno MUC1 were Generated for Antibody Generation, Screening, and Validation

Cell lines stably expressing human MUC1, including PT67/human MUC1 cell line (an internally generated cell line), HEK293/human MUC1 cell line (HEK293 is obtained from ATCC, CRL-1573), and HCT116/human MUC1 cell line (ATCC CCL-247) were generated and validated.

Cell lines stably expressing cyno MUC1 (SEQ ID NO: 3), including HEK293/cyno MUC1, L929/cyno MUC1 cell line (L929 was obtained from ATCC, CCL-1), HCT116/cyno MUC1 cell line, and Daudi/cyno MUC1 cell line (Daudi was obtained from ATCC, CCL-213), were generated and validated.

To generate cell lines that stably express human or cyno MUC1, ectotrophic vectors were constructed using retroviral construct PFBneo (STRATAGENE, cat. #217561-51). Retroviral construct transfection into PLAT-E cells (Cyagen, Cat. #IPMPC-01001) was performed using Lipofectamine 2000 (Invitrogen, REF. #52758) according to the manufacturer's instructions. Viral supernatants were collected 24, 48, and 72 hours after transfection and filtered (0.45 μm) before use. The ectotrophic viruses produced above were used to transduce the dual tropic packaging cell line PT67 in the presence of polybrene (final concentration: 8 μg/ml). After 3 rounds of transduction, PT67 cells were selected in G418 (final concentration: 1 mg/ml) for 7 days. For collection of dual tropic viruses generated from PT67 cells, when cells became 100% confluent, medium were changed to fresh DMEM complete medium without G418. Viruses were collected once a day for 3 days. Cell lines were infected with virus that contain human or cyno MUC. After 3 rounds of transduction, infected cells were selected in G418 (final concentration: 1 mg/ml) for 7 days.

### Immunization

To generate antibodies against MUC1, cohorts of 30 BALB/C, MRL strains of inbred mice were immunized with different MUC1 antigens with each cohort being subjected to an immunization strategy comprising a unique combination of MUC1 antigens (including the protein and cell line from Example 1), dose, injection route, adjuvant, and immunization timing. A total of 5 animals in 6 cohorts were immunized. Animals received immunizations over varying periods between 0 and 90 days. To monitor immune responses, titrated serum was screened by ELISA and FACS, typically after 30-90 days of 2-6 immunizations. Serum was screened for antibody binding to MUC1 antigens. MUC1-specific antibody responses were measured in each animal, and animals with sufficient titers of anti-MUC1 Ig were selected for final boost 4 days.

### Hybridoma Fusion and Screening

Lymphoid organs, including spleens and lymph nodes, were isolated from mice immunized as described above. Hybridomas were generated by fusions with immortalized mouse myeloma cells derived from the SP2/0 by PEG-based fusion. The resulting cells were plated in 96-well cell culture plates using regular 1640 medium supplemented with HAT for selection of hybridomas. After 10-13 days of culture and growth media replacement, hybridoma culture supernatants were collected from individual wells and screened to identify wells with secreted MUC1-specific antibodies. All supernatants were initially screened against recombinant proteins huMUC1-SEA-huIgG1 (from Example 1). Antibody binding on recombinant proteins huMUC1-SEA-huIgG1 were measured through ELISA. Supernatants from culture wells in 3 hybridoma fusions were screened for MUC1 antibodies. Briefly, 2 μg/mL huMUC1-SEA-huIgG1 was coated in the 96 well ELISA plates, and 50 μl of hybridoma culture supernatant were co-incubated for 30-60 min, washed, and incubated with anti-mouse IgG Fc secondary Ab conjugated to HRP. After incubation and washing, the plates were developed with HRP substrate and absorbance was measured.

Hybridomas from positive wells were transferred to 24-well plates with fresh culture media to grow for 2-3 days before screening again by flow cytometry to confirm antibodies binding to human MUC1 and cyno MUC1 overexpression cell lines.

Antibodies (Abs) binding to human MUC1 and cyno MUC1 overexpression cell lines were measured through FACS. Briefly, 100 μl of hybridoma culture supernatant and human MUC1 overexpression cells or cyno MUC1 overexpression cells were co-incubated for 30-60 min, washed, and incubated with anti-mouse IgG Fc secondary Ab conjugated to APC. After incubation and washing, fluorescence was measured by flow cytometry.

### Subcloning and Sequence Analysis

Selected anti-MUC1 Ab-secreting hybridomas were subcloned once or twice to ensure monoclonality. Briefly, the positive hybridoma clones were sub-cloned by limiting dilution. After 7-10 days, culture supernatant was screened by ELISA and flow cytometry as previously described to confirm human and cyno MUC1 Ab binding. Stable hybridoma subclones were cultured in vitro for cell cryopreservation and antibody VH and VL gene cloning and sequencing.

The anti-MUC1 Ab-secreting hybridomas after subcloning were lysed by lysis buffer. The mRNA-containing lysates were subsequently transferred to 96-well deep well plates for mRNA isolation, cDNA synthesis, and DNA sequencing by standard sequencing techniques (Sanger sequencing). In general, total RNA of the cell lysates was prepared and the cDNA was generated by reverse transcription of the mRNA using the Super Script III first-strand synthesis SuperMix (Invitrogen) according to the manufacturer's instructions. The sequences of the BG138P antibody are listed in Table 2.

### Single B Screening

Immunized mice were sacrificed and spleens were harvested. Enriched plasma cells were loaded onto 14K chip. hMUC1 beads, cynoMUC1 beads, and HEK293-cynoMUC1 cells were used for on-chip screening. Hits were selected and exported into lysis buffer. Single cell RNA was purified and Ig sequence were recovered using BL1 cDNA Synthesis Kit (BERKELEY LIGHTS) according to the manufacturer's instructions. The sequences of BG219P and BG346P are listed in Table 2.

TABLE 2
Sequences for murine antibodies against human MUC1

| Antibody | SEQ ID NO | Note | Sequence |
|---|---|---|---|
| BG138P | SEQ ID NO: 4 | HCDR1 | TYTMH |
| | | (Kabat) | |
| | SEQ ID NO: 5 | HCDR2 | YINPGSDYTHYNQKFKD |
| | | (Kabat) | |
| | SEQ ID NO: 6 | HCDR3 | QGNYAAYYFDY |
| | | (Kabat) | |
| | SEQ ID NO: 7 | LCDR1 | RASGNIHNFLV |
| | | (Kabat) | |
| | SEQ ID NO: 8 | LCDR2 | NAKTLTD |
| | | (Kabat) | |
| | SEQ ID NO: 9 | LCDR3 | QNFWNVPFT |
| | | (Kabat) | |
| | SEQ ID NO: 10 | VH AA | QVQLLQSGAELARPGASVKMSCKASGYT |
| | | | FTTYTMHWIKQRPGQGLEWIGYINPGSD |
| | | | YTHYNQKFKDKATLTADKSSSTAYMHLY |
| | | | SLTSEDSAVYYCARQGNYAAYYFDYWGQ |
| | | | GTTLTVSS |
| | SEQ ID NO: 11 | VL AA | DIQMTQSPASLSASVGETVTITCRASGNIH |
| | | | NFLVWYQQKQGKSPQLLVYNAKTLTDG |
| | | | VPSRFSGSGSGTQYSLKIKSLQPEDFGTY |
| | | | YCQNFWNVPFTFGSGTKLEIK |
| | SEQ ID NO: 12 | VH DNA | CAGGTCCAGTTGTTACAGTCTGGGGCTG |
| | | | AACTGGCAAGACCTGGGGCCTCAGTGA |
| | | | AGATGTCCTGCAAGGCTTCTGGTTACAC |
| | | | CTTTACTACCTACACGATGCACTGGATA |
| | | | AAACAGAGGCCTGGGCAGGGTCTGGAA |
| | | | TGGATTGGATACATTAATCCTGGCAGTG |
| | | | ATTATACTCATTACAATCAGAAATTCAAA |
| | | | GACAAGGCCACATTGACTGCAGACAAA |
| | | | TCCTCCAGCACAGCCTACATGCACCTGT |
| | | | ACAGCCTGACATCTGAGGACTCTGCAG |
| | | | TCTATTACTGTGCAAGACAGGGCAACTA |
| | | | TGCCGCGTACTACTTTGACTACTGGGGC |
| | | | CAAGGCACCACTCTCACAGTCTCCTCA |
| | SEQ ID NO: 13 | VL DNA | GACATCCAGATGACTCAGTCTCCAGCCT |
| | | | CCCTATCTGCATCTGTGGGAGAAACTGT |
| | | | CACCATCACATGTCGAGCAAGTGGGAAT |
| | | | ATTCACAATTTTTTAGTATGGTATCAGCA |
| | | | GAAACAGGGAAAATCTCCTCAGCTCCT |
| | | | GGTCTATAATGCAAAAACCTTAACAGAT |
| | | | GGTGTGCCATCAAGGTTCAGTGGCAGT |
| | | | GGATCAGGAACACAATATTCTCTCAAGA |
| | | | TCAAGAGCCTGCAGCCTGAAGATTTTG |
| | | | GGACTTATTACTGTCAAAATTTTTGGAAT |
| | | | GTTCCATTCACGTTCGGCTCGGGGACAA |
| | | | AGTTGGAAATAAAA |
| BG346P | SEQ ID NO: 14 | HCDR1 | NYWMN |
| | | (Kabat) | |
| | SEQ ID NO: 15 | HCDR2 | EIDPSDSYTNNNQKFKD |
| | | (Kabat) | |
| | SEQ ID NO: 16 | HCDR3 | GRRTGFAY |
| | | (Kabat) | |
| | SEQ ID NO: 17 | LCDR1 | RASENIYSNLA |
| | | (Kabat) | |
| | SEQ ID NO: 18 | LCDR2 | AATNLAD |
| | | (Kabat) | |
| | SEQ ID NO: 19 | LCDR3 | QHFWGTPYT |
| | | (Kabat) | |
| | SEQ ID NO: 20 | VH AA | EVQLQQSGAELVKPGASVKISCKASGYTF |
| | | | TNYWMNWVRQRPGQGLEWIGEIDPSDS |
| | | | YTNNNQKFKDKATLTVDKSSSTAYMQLS |
| | | | SLTSEDSAVYYCARGRRTGFAYWGQGTL |
| | | | VTVSS |
| | SEQ ID NO: 21 | VL AA | DIQMTQSPASLSVSVGETVTITCRASENIY |
| | | | SNLAWYQKKQGKSPQLLVYAATNLADGV |
| | | | PSRFSGSGSGTQYSLKINSLQSEDFGNYY |
| | | | CQHFWGTPYTFGGGTKLEIK |
| | SEQ ID NO: 22 | VH DNA | GAGGTCCAGCTTCAGCAGTCTGGGGCT |
| | | | GAGCTGGTGAAGCCTGGGGCTTCAGTG |
| | | | AAGATATCCTGCAAGGCGTCTGGCTACA |
| | | | CGTTCACCAACTACTGGATGAACTGGGT |
| | | | GAGGCAGAGGCCTGGACAAGGCCTTGA |
| | | | GTGGATCGGAGAGATCGATCCTTCTGAT |
| | | | AGTTATACTAACAACAATCAAAAGTTCA |
| | | | AGGACAAGGCCACATTGACTGTAGACA |
| | | | AATCCTCCAGCACAGCCTACATGCAGCT |
| | | | CAGCAGCCTGACATCTGAGGACTCTGC |
| | | | GGTCTATTACTGTGCAAGAGGGAGGAG |
| | | | GACGGGGTTTGCTTACTGGGGCCAAGG |
| | | | GACTCTGGTCACTGTCTCGAGC |
| | SEQ ID NO: 23 | VL DNA | GACATCCAGATGACCCAGTCTCCAGCCT |
| | | | CCCTATCTGTATCTGTGGGAGAAACTGT |
| | | | CACCATCACATGTCGAGCAAGTGAGAAT |
| | | | ATTTACAGTAATTTAGCATGGTATCAGAA |
| | | | GAAACAGGGAAAATCTCCTCAGCTCCT |
| | | | GGTCTATGCTGCAACAAACTTAGCAGAT |
| | | | GGTGTGCCATCAAGGTTCAGTGGCAGT |
| | | | GGATCAGGCACACAGTATTCCCTCAAGA |
| | | | TCAACAGCCTGCAGTCTGAAGATTTTGG |
| | | | GAATTATTACTGTCAACATTTTTGGGGTA |
| | | | CTCCGTACACGTTCGGAGGGGGGACCA |
| | | | AGCTGGAAATAAAA |
| BG219P | SEQ ID NO: 24 | HCDR1 | DYIMS |
| | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 28 | LCDR2 | NAKTLAD |
| | | (Kabat) | |
| | SEQ ID NO: 29 | LCDR3 | QHFWSVPYT |
| | | (Kabat) | |
| | SEQ ID NO: 30 | VH AA | EVOLVESGGGLVKPGGSLKLSCAASGFTF |
| | | | NDYIMSWVRQTPEKRLEWVATVSSGGDN |
| | | | TYYPDSVKGRFTISRDNAKNNLYLQMSSL |
| | | | RSEDTALYYCTRYGGLGAMDYWGQGTS |
| | | | VTVSS |
| | SEQ ID NO: 31 | VL AA | DIVMTQSPASLSASVGETVTITCRASGNIH |
| | | | NYLTWYQQKQGKSPQLLVYNAKTLADG |
| | | | VPSRFSGSGSGPQYSLKINSLQPEDFGSYY |
| | | | CQHFWSVPYTFGGGTRLEIK |
| | SEQ ID NO: 32 | VH DNA | GAAGTGCAGCTGGTGGAGTCTGGGGGA |
| | | | GGCTTAGTGAAGCCTGGAGGGTCCCTG |
| | | | AAACTCTCCTGTGCAGCCTCTGGATTCA |
| | | | CTTTCAATGACTATATCATGTCTTGGGTT |
| | | | CGCCAGACTCCGGAGAAGAGGCTGGAG |
| | | | TGGGTCGCAACCGTTAGTAGTGGTGGTG |
| | | | ATAACACCTACTATCCAGATAGTGTGAA |
| | | | GGGTCGATTCACCATCTCCAGAGACAAT |
| | | | GCCAAGAACAACCTGTACCTGCAAATG |
| | | | AGCAGTCTGAGGTCTGAGGACACGGCC |
| | | | TTGTATTACTGTACAAGATATGGGGGGTT |
| | | | AGGGGCTATGGACTACTGGGGTCAAGG |
| | | | AACCTCAGTCACCGTCTC |
| | SEQ ID NO: 33 | VL DNA | GACATTGTGATGACCCAGTCTCCAGCCT |
| | | | CCCTATCTGCATCTGTGGGAGAAACTGT |
| | | | CACCATCACATGTCGAGCAAGTGGGAAT |
| | | | ATTCACAATTATTTAACATGGTATCAGCA |
| | | | GAAACAGGGAAAATCTCCTCAGCTCCT |
| | | | GGTCTATAATGCAAAAACCTTAGCAGAT |
| | | | GGTGTGCCATCAAGGTTCAGTGGCAGT |
| | | | GGATCAGGACCACAATATTCTCTCAAGA |
| | | | TCAACAGCCTGCAGCCTGAAGATTTTGG |
| | | | GAGTTATTACTGTCAACATTTTTGGAGT |
| | | | GTTCCGTACACGTTCGGAGGGGGGACC |
| | | | AGACTGGAAATAAAA |

### Large Scale Expression and Purification of Chimeric BG219P, BG138P, And BG346P

The chimeric antibodies chBG219P, chBG138P. and chBG346P were produced by transient transfection of in-house generated heavy and light chain containing plasmids to ExpiCHO-s cells. The conditioned media was harvested and the antibodies were purified using a MabSelect SuRe column (Cytiva) followed by a POROS™ 50 HS column (Thermofisher Scientific) and a G-25 desalting column (Cytiva). All the purified antibodies were stored in a −80° C. freezer in small aliquots.

### Example 2. Binding Kinetics and Affinity Determination of Anti-MUC1 Antibodies by SPR

Chimeric anti-MUC1 antibodies were characterized for binding kinetics by SPR assays using BIAcore™ T-200 (GE Life Sciences). Briefly, mouse anti-human IgG Fc antibody was immobilized on an activated CM5 biosensor chip (Cat. No. BR100530, GE Life Sciences). Purified chimeric anti-MUC1 antibodies were flowed over the chip surface and captured by anti-human IgG antibody. Then a serial dilution of human or cyno MUC1-SEA protein was flowed over the chip surface and changes in surface plasmon resonance signals were analyzed to calculate the association rates (kₒₙ) and dissociation rates (k_{off}) by using the one-to-one Langmuir binding model (BIA Evaluation Software, GE Life Sciences). The equilibrium dissociation constant (K_{D}) was calculated as the ratio k_{off}/kₒₙ. The binding affinity profiles of chimeric anti-MUC1 antibodies chBG1138P, chBG346P, and chBG219P are shown below in Table 3. ChBG138P, chBG346P. and chBG219P displayed high affinity towards both human and cyno MUC1-SEA.

TABLE 3
Antigen binding affinity of chBG138P, chBG346P, and ch BG219P

| Ab# | Antigen | ka (1/Ms) | kd (1/s) | KD (M) |
|---|---|---|---|---|
| chBG138P | HuMUC1-SEA | 2.37E+05 | 4.37E−04 | 1.85E−09 |
| | CynoMUC1-SEA | 6.38E+05 | 9.75E−03 | 1.53E−08 |
| chBG346P | HuMUC1-SEA | 7.97E+05 | 7.59E−04 | 9.53E−10 |
| | CynoMUC1-SEA | 2.67E+05 | 3.35E−04 | 1.26E−09 |
| chBG219P | HuMUC1-SEA | 3.39E+05 | 1.25E−04 | 3.69E−10 |
| | CynoMUC1-SEA | 3.52E+05 | 3.29E−04 | 9.34E−10 |

### Example 3. Determining the Binding Affinity of Anti-MUC1 Antibodies to MUC1 Expressed in Stable Expression Cell Lines

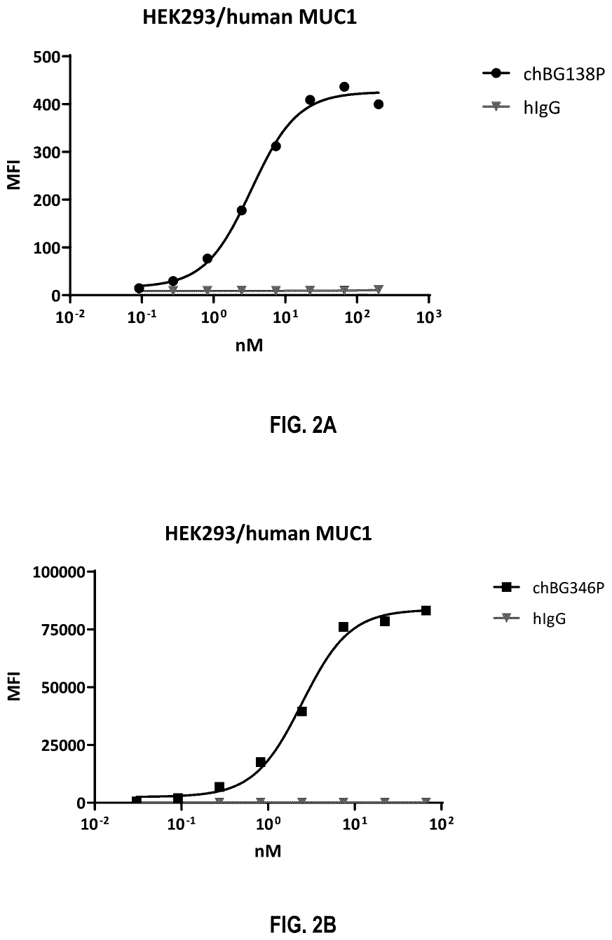

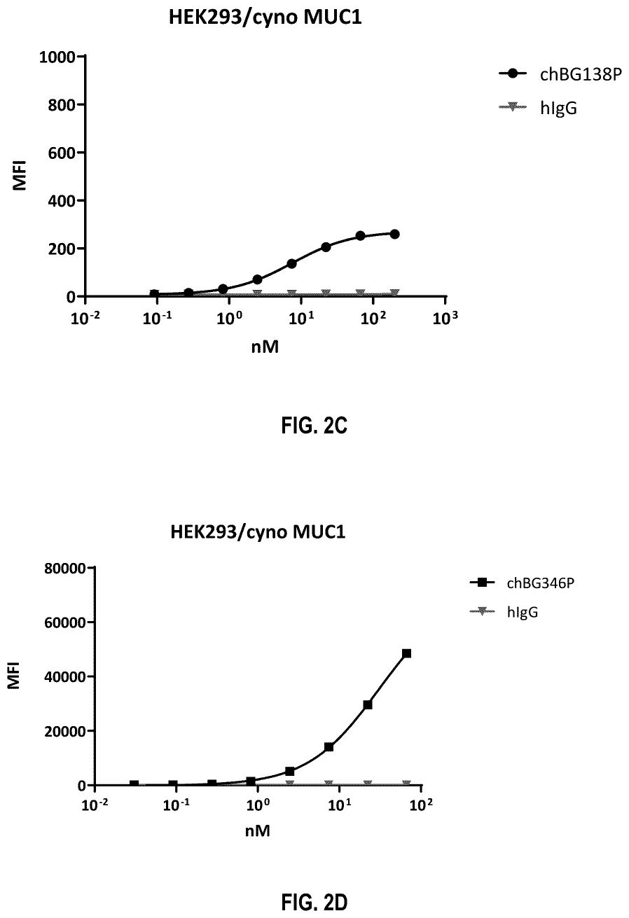

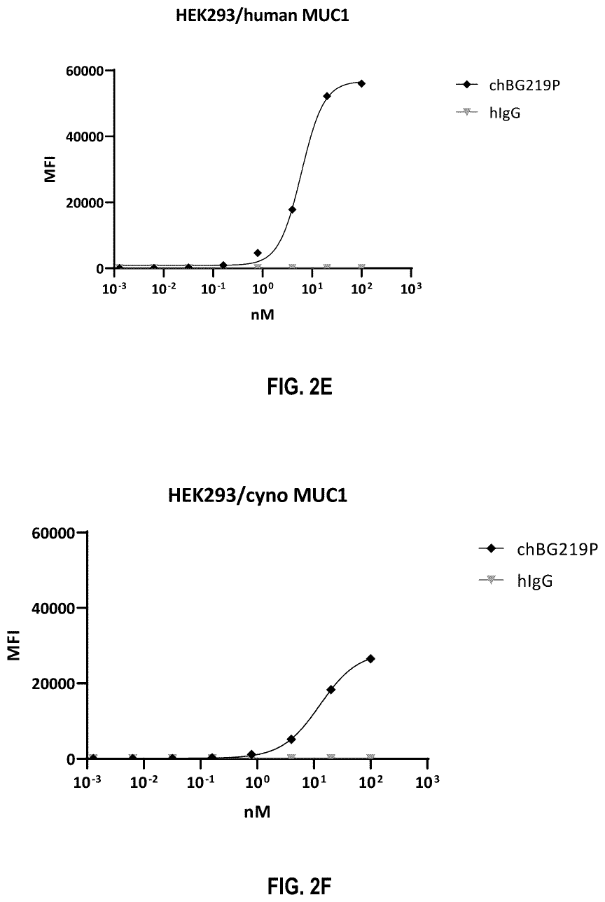

The binding affinity of chimeric anti-MUC1 antibodies to human and cynoMUC1 overexpression cell lines (HEK293/human MUC1 and HEK293/cyno MUC1) by FACS was determined. Briefly, human or cyno MUC1 overexpression cells were incubated with serially diluted purified antibodies, washed, and incubated with anti-human IgG secondary antibody conjugated to APC. After incubation and washing, fluorescence was measured by flow cytometry. The binding affinity profiles of chimeric anti-MUC1 antibodies are shown below in Table 4 and FIGS. 2A-2F (with hIgG11 as negative control). The results demonstrate that all three chimeric anti-MUC1 antibodies have favorable binding affinity to both human and cyno MUC1 expressed in stable expression cell lines.

TABLE 4
Cell binding affinity of chBG138P, chBG346P, and chBG219P

| Ab# | Cell line | EC₅₀ (nM) |
|---|---|---|
| chBG138P | Human MUC1 | 3.37 |
| | Cyno MUC1 | 7.55 |
| chBG346P | Human MUC1 | 3.01 |
| | Cyno MUC1 | 41.76 |
| chBG219P | Human MUC1 | 6.06 |
| | Cyno MUC1 | 12.71 |

### Example 4. Epitope Binning of Anti-MUC1 Antibodies

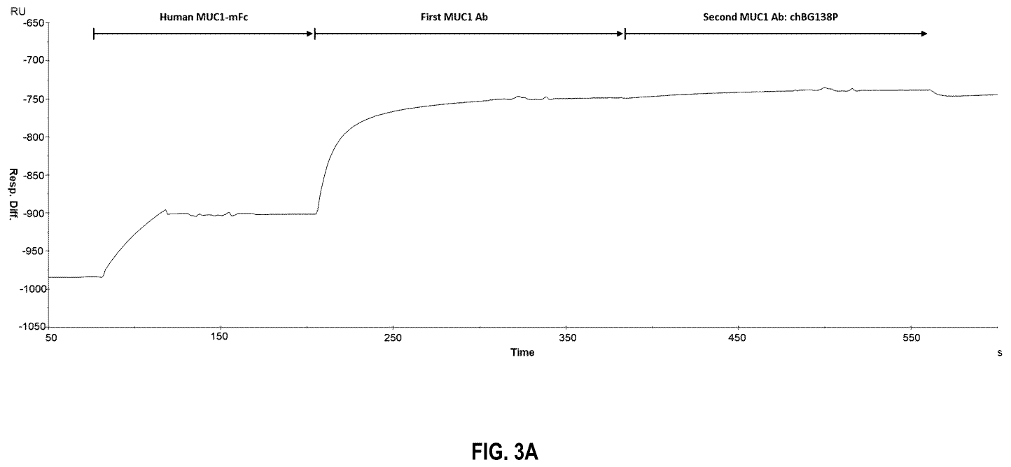

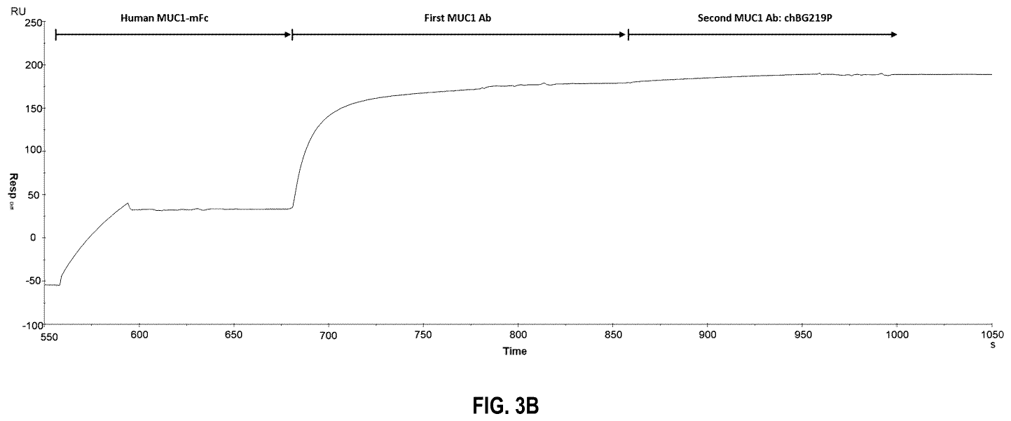

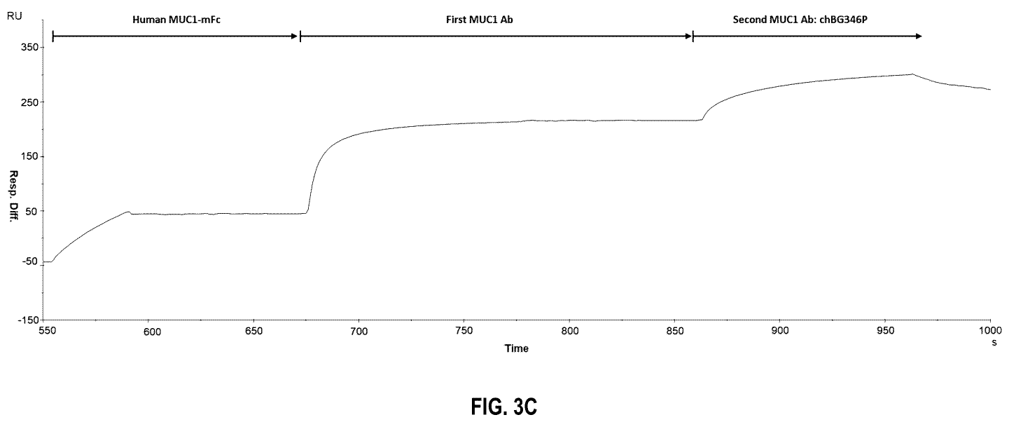

The epitope binning of chimeric anti-MUC1 antibodies was determined by a competitive SPR assay. Briefly, anti-mouse IgG Fc antibody was immobilized on an activated CM5 biosensor chip. Purified huMUC1-SEA-mIgG2a (human MUC1 linked with mouse IgG2a Fc) antigen was flowed over the chip surface and captured by anti-mouse IgG antibody. The reference MUC1-SEA Ab 5F3 (Cancer Immunol Immunother. 2020 July; 69(7):1337-1352) was first injected at saturating antigen binding conditions, followed by injection of chBG138P (FIG. 3A), chBG219P (FIG. 3B), or chBG346P (FIG. 3C). The sensorgrams for epitope binning are shown in FIGS. 3A-3C. As shown in Table 5 below, the three chimeric MUC1 antibodies were grouped into two epitope bins in the MUC1-SEA domain. ChBG138P and chBG219P bind to the same epitope on MUC1-SEA, as does 5F3, which is a different MUC1 epitope from chBG346P. More specifically, chBG138P and chBG219P bind to epitope bin A, while chBG346P binds to bin B.

TABLE 5
Epitope bins of chBG138P, ch BG346P, and chBG219P

| Ab# | Bin |
|---|---|
| chBG138P | A |
| chBG346P | B |
| chBG219P | A |

### Example 5. Chimeric Anti-MUC1 Antibodies chBG138P, chBG219P, and chBG346P Show Reduced Interference by Soluble MUC1

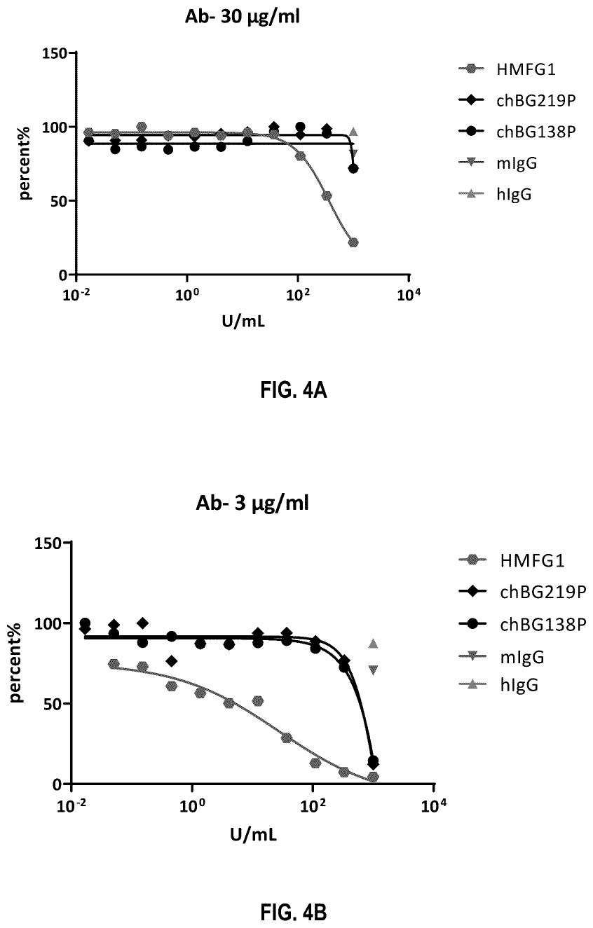

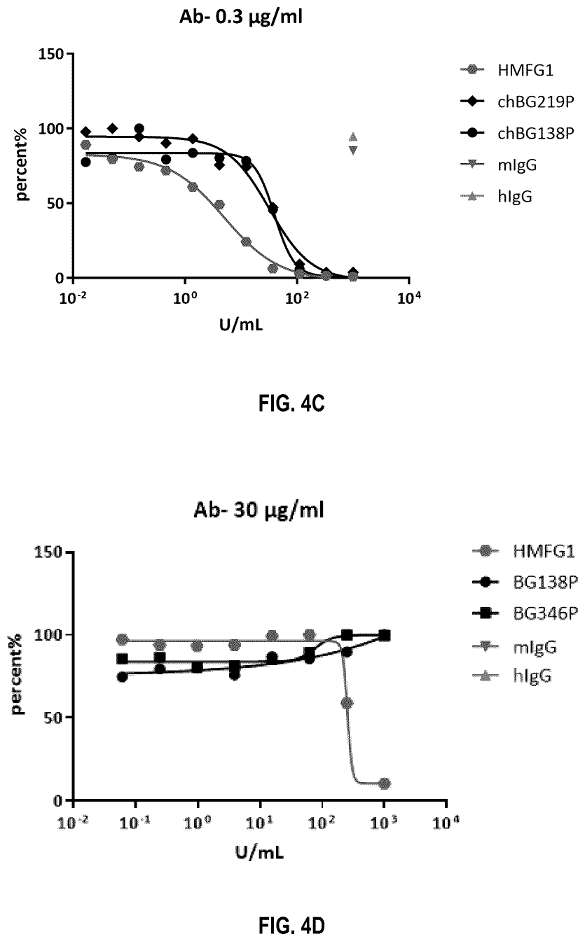

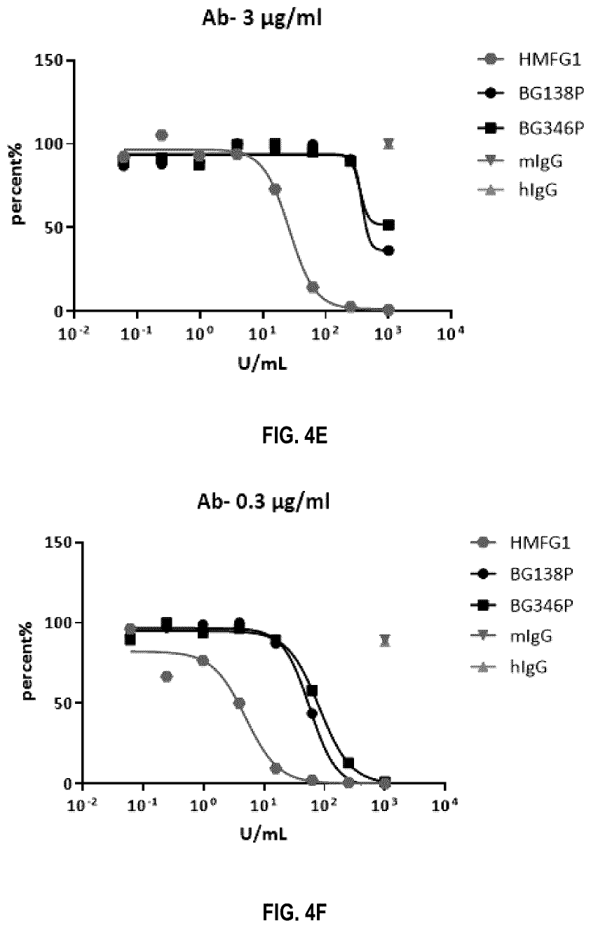

The presence of soluble MUC1 on the specific binding of MUC1 antibodies to MUC1 expressing cells was determined by competitive FACS assay. Briefly, human MUC1 expressing cells were incubated with 30, 3, and 0.3 μg/ml MUC1 antibodies in the presence of serially diluted soluble MUC1 (Shanghai Linc-Bio Science Co. LTD). After washing and incubation with anti-human IgG secondary antibody, fluorescence was measured by flow cytometry. IC₅₀ values for soluble MUC1 blocking MUC1 antibodies binding with MUC1 expressing cells is shown in Table 6, and the blocking curves are shown in FIGS. 4A-4F, in which HMFG1 is a positive control, and mIgG and higG1 are negative controls. The profiles indicate that the binding of HMFG to MUC1-expressing cells can be easily interfered with at high, medium, and low antibody concentrations (i.e., 30, 3, 0.3 μg/ml), but chBG138P, chBG219P, and chBG346P binding is only slightly interfered with at the low antibody concentration (i.e., 0.3 μg/ml) (FIGS. 4A-F). Collectively, the profiles in FIG. 4 indicate that the binding of MUC1 antibodies to MUC1-expressing cells showed significantly reduced interference by soluble MUC1 compared to HMFG1 (Abcam), which targets the MUC1 N-terminal.

TABLE 6
IC₅₀ of soluble MUC1 blocking activity
IC₅₀ (U/mL)

| Antibody | 30 μg/ml | 3 μg/ml | 0.3 μg/ml |
|---|---|---|---|
| chBG138P | N.A. | N.A. | 56.8 |
| chBG346P | N.A. | N.A. | 82.1 |
| chBG219P | N.A. | N.A. | 31.8 |

(N.A.: Not available. IC₅₀ data cannot be fitted well and retrieved due to the extremely low interference, as shown from the binding curves in FIGS. 4A, 4B, 4E, and 4F.)

### Example 6. Anti-MUC1 Monoclonal Antibodies Targeting MUC1 Membrane Proximal Region Bind to Cancer Cell Lines but do not Bind to Normal T Cells, while MUC1 N-Terminal Targeting Antibodies HMFG1 or 16A can Bind to Normal T Cells

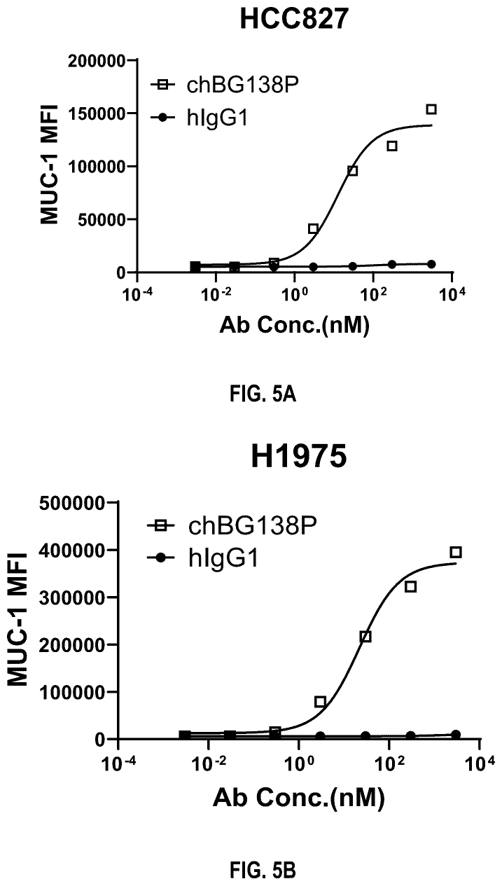

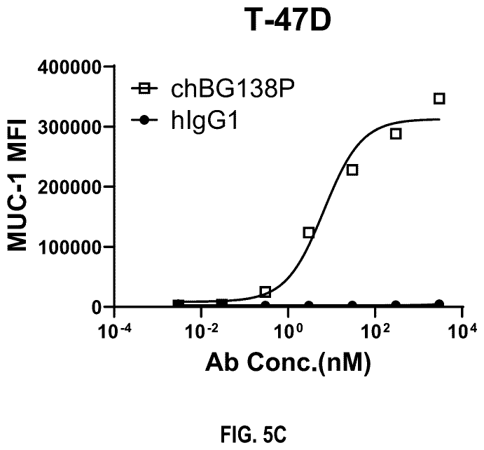

To evaluate whether the anti-MUC1 monoclonal antibodies targeting MUC1 membrane proximal region can differentially bind to MUC1 expressing tumor cells versus MUC1 expressing normal cells, like activated T cells, FACS binding assays were performed. For tumor cell line binding experiments, cells stained with anti-human MUC1 antibody chBG138P or controls (human IgG1) for 1 hour were collected. Then cells were washed twice and followed by staining with a secondary antibody (Alexa Fluor®647 anti-human IgG Fc) for 30 minutes. Cells were washed and fixed with 1% paraformaldehyde (PFA) in DPBS before FACS analysis. All flow cytometry data were acquired using NovoCyte flow cytometer (ACEA Biosciences, Inc.) and data was analyzed using NovoExpress software. As shown in FIGS. 5A-5C, the chimeric antibody chBG138P, which targets the human MUC1 membrane proximal region, binds to MUC1 expressing tumor cell lines HCC827 (FIG. 5A), H1975 (FIG. 5B), and T-47D (FIG. 5C) in a dose dependent manner (human IgG1 as a negative control), indicating that the MUC1 membrane proximal region targeting antibodies can be used to target cancer cells and thus be applied to treat cancers that express MUC1.

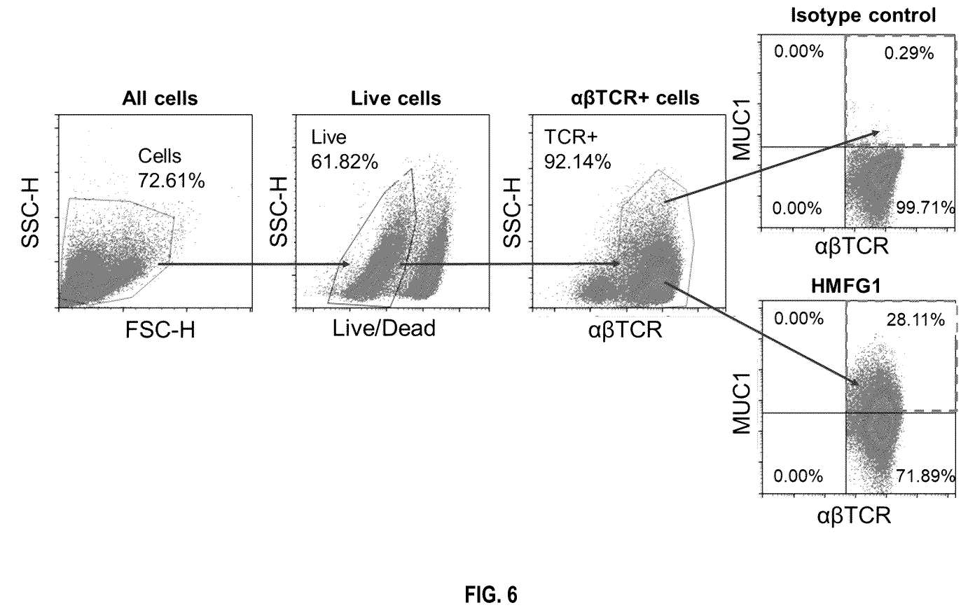

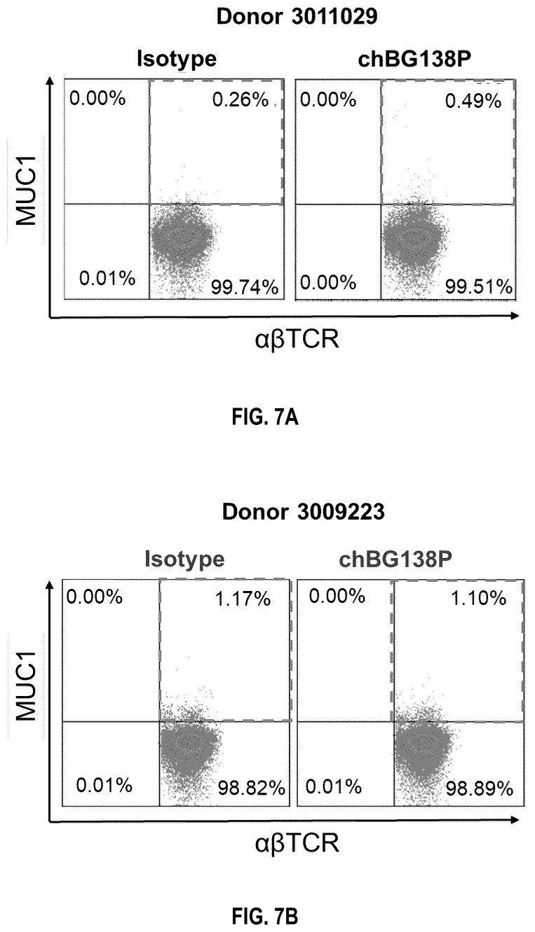

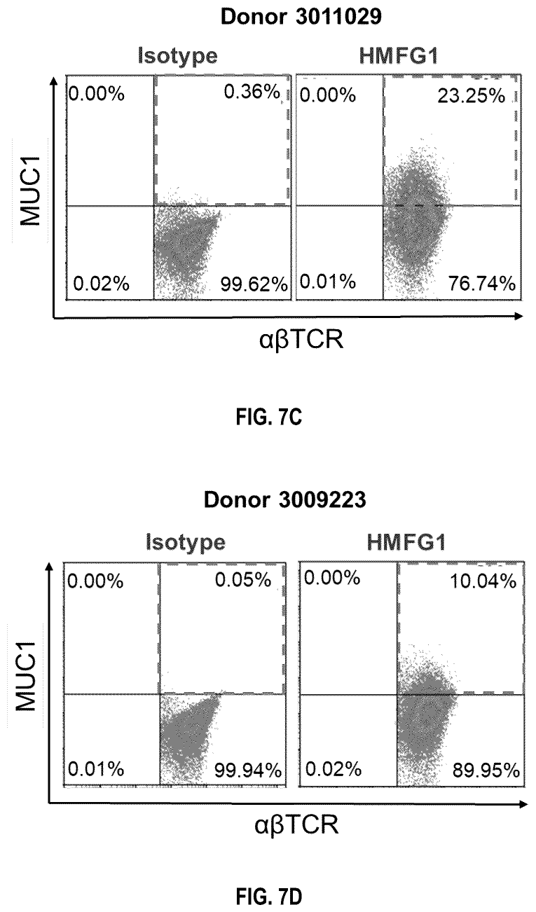

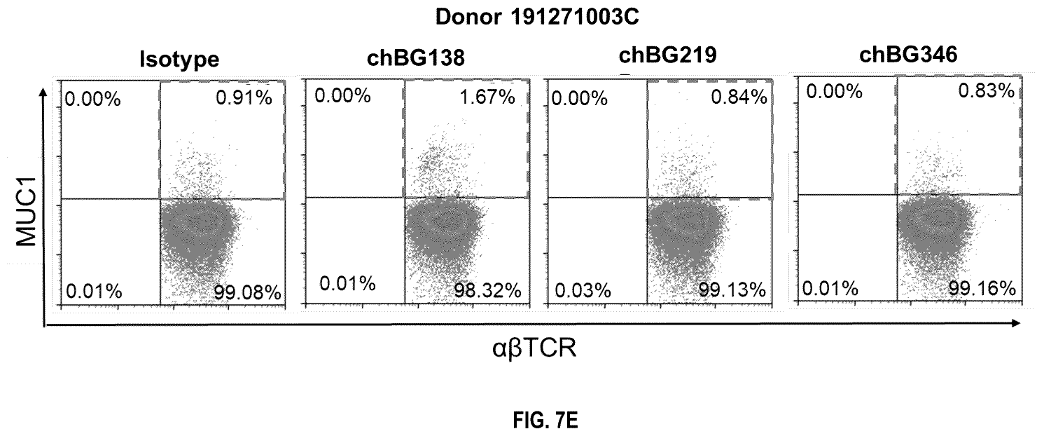

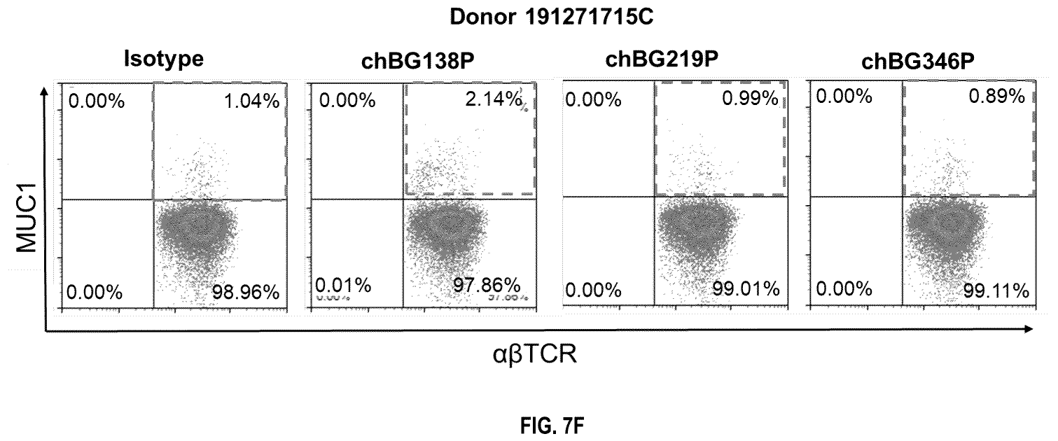

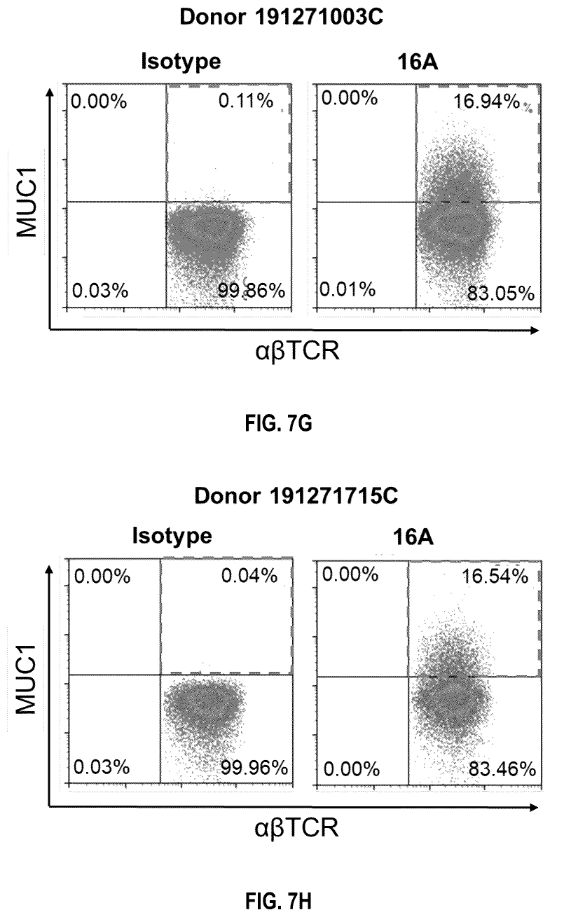

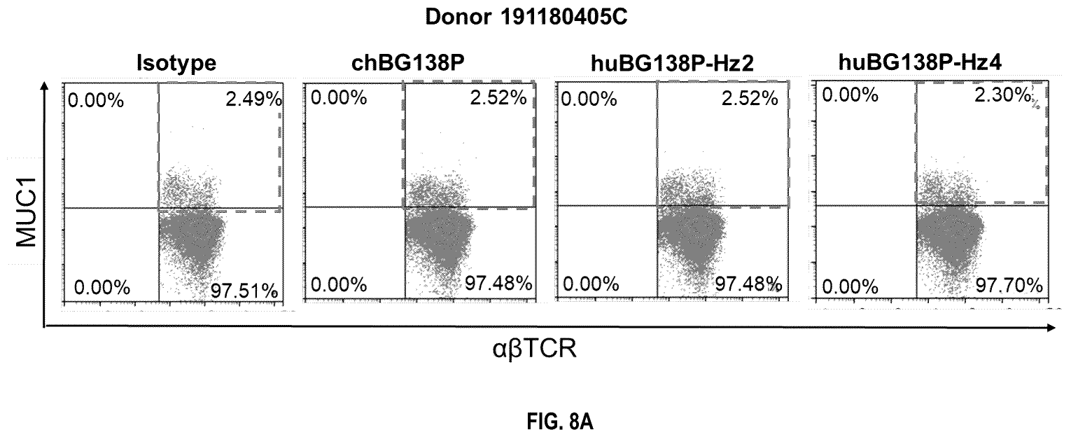

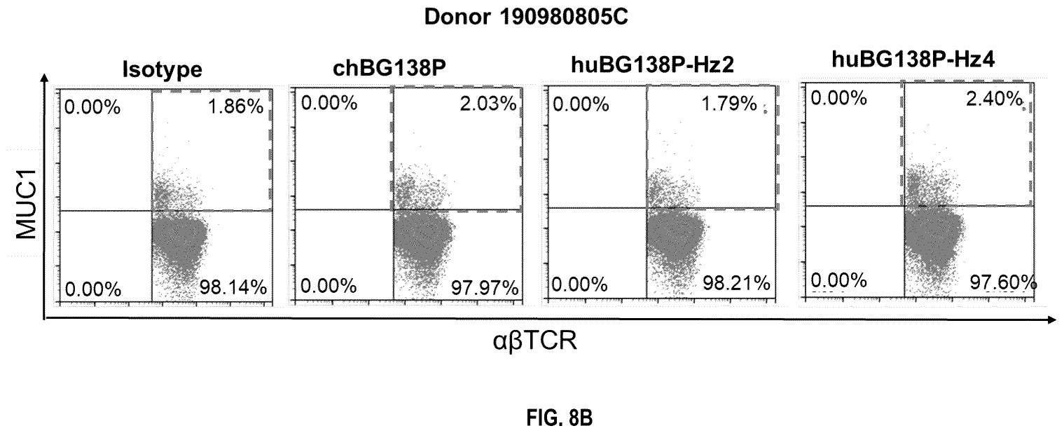

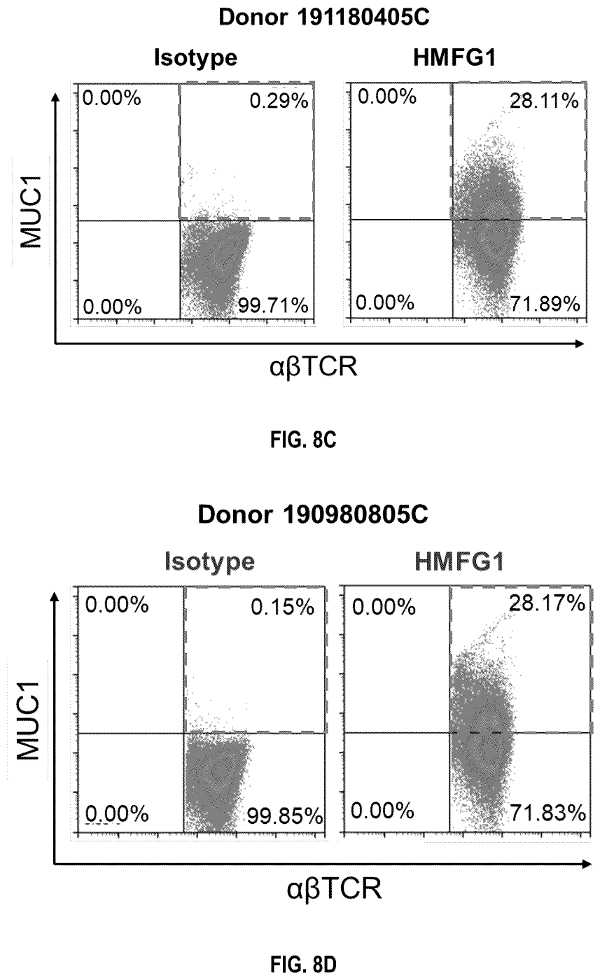

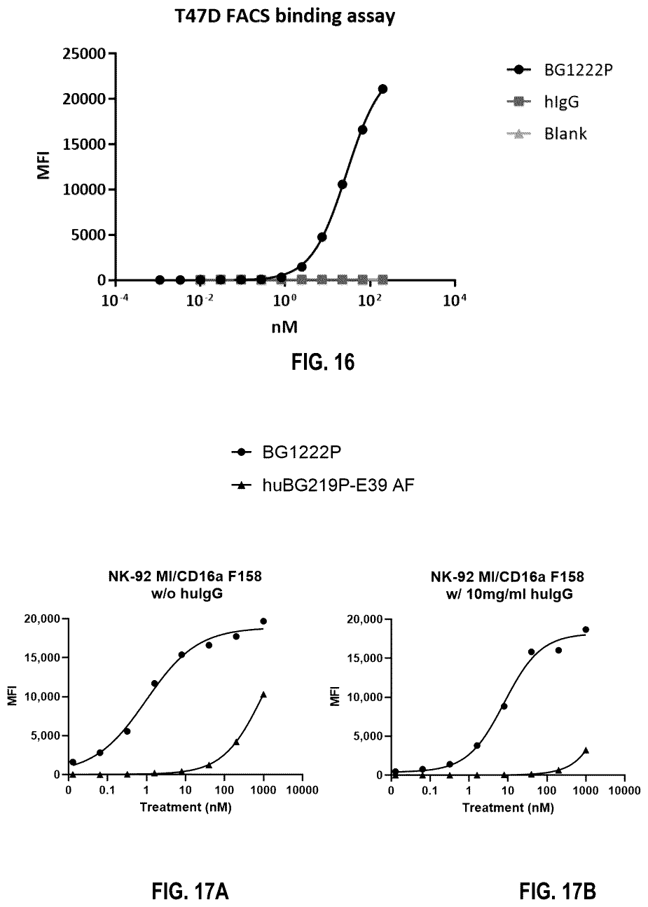

To evaluate whether the monoclonal antibodies targeting MUC1 membrane proximal region can avoid binding to normal cells that express MUC1, an activated T cell binding assay was conducted. Briefly, human peripheral blood mononuclear cells (PBMCs) from six healthy donors purchased from Allcells or Stemcell were stimulated with 1 μg/ml PHA-L for 3 days. Then FACS staining was performed using the stimulated PBMCs. Cell suspensions were pre-incubated with LIVE/DEAD™ Fixable Dead Cell Stain Kit (Invitrogen, REF. #L34964) and Fc receptor blocking solution (100 μg/mL human IgG in FACS buffer), before staining with anti-human antibodies. Cells were washed twice and incubated with 10 μg/mL anti-MUC1 monoclonal antibodies targeting MUC1 membrane proximal region or MUC1 N-terminal targeting antibodies HMFG1 (as positive control, Abcam, cat. #ab215670) or 16A (as positive control, Biolegend, cat. #355608) for 1 hour. Then cells were washed and stained with PE-CY7 anti-human αβ TCR (eBioscience, cat. #25-9986-42), AF647 anti-human IgG Fc (Biolegend, REF. #409320) for 30 minutes. Cells were washed and fixed with 1% PFA in DPBS before FACS analysis. All flow cytometry data were acquired using NovoCyte flow cytometer (ACEA Biosciences, Inc.) and data was analyzed using NovoExpress software. As shown in FIGS. 6 and 7A-7H, none of the chimeric antibodies chBG138P (FIGS. 7A, 7B, 7E, and 7F), chBG219P (FIGS. 7E and 7F), or chBG346P (FIGS. 7E and 7F) binds to normal activated human T cells that express MUC1. However, the MUC1-N terminal targeting antibodies HMFG1 (FIGS. 7C and 7D) and 16A (FIGS. 7G and 7H) bind to a significant portion of activated T cells. FIGS. 8A-8D show that the chimeric (chBG138P) and humanized (huBG138P-Hz2 and huBG138P-Hz4) versions of the antibody BG138P retain similar binding properties with BG138P with no binding to normal activated human T cells that express MUC1 (FIGS. 8A and 8B), while MUC1-N terminal targeting antibody HMFG1 (FIGS. 8C and 8D) binds to normal activated T cells.

The results indicate that. compared to MUC1-N terminal targeting antibodies that bind to both cancer cells and normal T cells, antibodies targeting MUC1 membrane proximal region specifically target cancer cells while sparing normal T cells, and thus may confer an optimized safety profile when used as an anti-tumor therapy in humans.

### Example 7. Humanization of the Murine Anti-Human MUC1 Antibody BG219P

For humanization of BG219P, human germline IgG genes were searched for sequences that share high degrees of homology to the protein sequences of BG219P variable regions by sequence comparison against the human immunoglobulin gene database in IMGT. The human IGHV and IGKV genes that are present in human antibody repertoires with high frequencies and highly homologous to murine BG219P were selected as the templates for humanization.

Humanization was carried out by CDR-grafting followed by critical back mutations being incorporated. The humanized antibodies were engineered as human IgG1 wild type format by using an in-house developed expression vector. In the initial round of humanization, mutations from murine to human amino acid residues in framework regions were guided by 3D structure analysis and the murine framework residues with structural importance for maintaining the canonical structural of CDRs being retained in the first round of the humanization design. BG219P-Bz0, which is the antibody variant of CDRs graft version with all back mutation sites, among all the 19 variants generated, is the variant with theoretical binding capacity approximate to parental murine antibody BG219P.

Specifically, BG219P-Bz0 was generated as described herein. Human germline variable gene IGKV1-39*01 and IGKJ2*01 and human germline variable gene IGHV3-23*01 and IGHJ6*01 were selected as receptor frameworks for BG219P VL and VH sequences. LCDRs of murine BG219P were grafted into the framework of human germline variable gene IGKV1-39*01 and IGKJ2*01 with D17E, A43S, 148V, T69P, and F71Y murine framework residues. The amino acid sequence and DNA sequence of the resulting BG219P-Bz0 VL are shown in Table 8. HCDRs of murine BG219P were grafted into the framework of human germline variable gene IGHV3-23*01 and IGHJ6*01 with S30N, S49A, A93T, and K94R murine framework residues retained. The amino acid sequence and DNA sequence of the resulting BG219P-Bz0 VH are shown in Table 8.

Beginning with the humanized BG219P antibody huBG219P-Bz0, several additional amino acid changes in the CDR region of both VH and VL were made to further improve the biophysical properties for therapeutic use in humans. The considerations included removing post-translational modifications and improving heat stability (Tm) while maintaining binding activities.

More than thirty humanized BG219P (also referred as huBG219P) variants were constructed with in-house IgG1/Cκ eukaryotic expression vectors that contain constant regions of a human wildtype IgG1 and kappa chain, respectively, with easy adapting sub-cloning sites. The variants were produced by transient transfection of the plasmids into ExpiCHO-s cells (Thermofisher Scientific). The conditioned medium was harvested and the variants were purified using a MabSelect™ SuRe column (Cytiva) followed by UF/DF to change the buffer. All the purified antibodies were stored in a −80° C. freezer in small aliquots.

For affinity determination, antibodies were captured by anti-human Fc surface, and used in the affinity assay based on surface plasmon resonance (SPR) technology. The results of SPR-determined binding profiles of anti-MUC1 antibodies are summarized in Table 7. huBG2.9P-E39- and huBG29P-E43 have similar binding affinities with dissociation constants at 35.2 pM and 30.3 pM. respectively, which are comparable to that of chimeric BG219P (39.8 pM). The sequences of huBG219P-E39 and huBG219P-E43 are provided in Table 8.

TABLE 7
Comparison of huBG219P binding affinities to
MUC1 SEA-Fc by SPR

| Antibody | Kₒₙ (M−1s−1) | K_{off} (s−1) | K_{D} (nM) |
|---|---|---|---|
| chBG219P | 1.44E+06 | 5.74E−05 | 3.98E−011 |
| huBG219P-E39 | 1.40E+06 | 4.92E−05 | 3.52E−011 |
| huBG219P-E43 | 1.61E+06 | 4.86E−05 | 3.03E−011 |

TABLE 8
Sequence Table

| Antibody | SEQ ID NO | | SEQUENCE |
|---|---|---|---|
| huBG219P- | SEQ ID NO: 24 | HCDR1 | DYIMS |
| Bz0 | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 28 | LCDR2 | NAKTLAD |
| | | (Kabat) | |
| | SEQ ID NO: 29 | LCDR3 | QHFWSVPYT |
| | | (Kabat) | |
| | SEQ ID NO: 61 | VH AA | EVQLLESGGGLVQPGGSLRLSCAASGFT |
| | | | FNDYIMSWVRQAPGKGLEWVATVSSG |
| | | | GDNTYYPDSVKGRFTISRDNSKNTLYLQ |
| | | | MNSLRAEDTAVYYCTRYGGLGAMDYW |
| | | | GQGTLVTVSS |
| | SEQ ID NO: 62 | VL AA | DIQMTQSPSSLSASVGERVTITCRASGNI |
| | | | HNYLTWYQQKPGKSPKLLVYNAKTLA |
| | | | DGVPSRFSGSGSGPDYTLTISSLQPEDFA |
| | | | TYYCQHFWSVPYTFGQGTKLEIK |
| | SEQ ID NO: 63 | VH DNA | GAAGTGCAGCTGCTCGAGAGCGGGGG |
| | | | CGGCCTCGTGCAACCTGGCGGGAGCC |
| | | | TGAGACTGAGCTGCGCCGCTAGCGGC |
| | | | TTCACCTTCAACGACTACATCATGAGC |
| | | | TGGGTGAGACAAGCCCCCGGCAAGGG |
| | | | CCTGGAGTGGGTGGCCACCGTGAGCA |
| | | | GCGGGGGCGACAACACCTACTACCCC |
| | | | GACAGCGTGAAGGGCAGATTCACCAT |
| | | | CAGCAGAGACAACAGCAAGAACACCC |
| | | | TGTACCTGCAGATGAACAGCCTGAGA |
| | | | GCCGAGGACACCGCCGTGTACTACTG |
| | | | CACAAGATACGGCGGCCTGGGCGCCA |
| | | | TGGACTACTGGGGCCAAGGCACCCTG |
| | | | GTGACCGTCAGCTCC |
| | SEQ ID NO: 64 | VL DNA | GACATTCAGATGACACAGAGCCCTAG |
| | | | CAGCCTGAGCGCTAGCGTGGGCGAGA |
| | | | GAGTGACCATCACCTGCAGAGCTAGC |
| | | | GGCAACATCCACAACTACCTGACCTG |
| | | | GTATCAGCAGAAGCCCGGCAAGAGCC |
| | | | CCAAGCTGCTGGTGTACAACGCCAAG |
| | | | ACCCTGGCCGACGGCGTGCCTAGCAG |
| | | | ATTCAGCGGCAGCGGCAGCGGCCCCG |
| | | | ACTACACCCTGACCATCAGCAGCCTGC |
| | | | AGCCCGAGGACTTCGCCACCTACTACT |
| | | | GTCAGCACTTCTGGAGCGTGCCCTACA |
| | | | CCTTCGGCCAAGGCACCAAGCTGGAG |
| | | | ATCAAG |
| huBG219P- | SEQ ID NO: 24 | HCDR1 | DYIMS |
| E39 | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 61 | VH | EVQLLESGGGLVQPGGSLRLSCAASGFT |
| | | AA | FNDYIMSWVRQAPGKGLEWVATVSSGG |
| | | | DNTYYPDSVKGRFTISRDNSKNTLYLQM |
| | | | NSLRAEDTAVYYCTRYGGLGAMDYWG |
| | | | QGTLVTVSS |
| | SEQ ID NO: 66 | VL | DIQMTQSPSSLSASVGDRVTITCRASGNI |
| | | AA | HNYLTWYQQKPGKAPKLLIYNAKTLAS |
| | | | GVPSRFSGSGSGTDFTLTISSLQPEDFATY |
| | | | YCQHFWSVPYTFGQGTKLEIK |
| | SEQ ID NO: 63 | VH | GAAGTGCAGCTGCTCGAGAGCGGGGG |
| | | DNA | CGGCCTCGTGCAACCTGGCGGGAGCC |
| | | | TGAGACTGAGCTGCGCCGCTAGCGGC |
| | | | TTCACCTTCAACGACTACATCATGAGC |
| | | | TGGGTGAGACAAGCCCCCGGCAAGGG |
| | | | CCTGGAGTGGGTGGCCACCGTGAGCA |
| | | | GCGGGGGCGACAACACCTACTACCCC |
| | | | GACAGCGTGAAGGGCAGATTCACCAT |
| | | | CAGCAGAGACAACAGCAAGAACACCC |
| | | | TGTACCTGCAGATGAACAGCCTGAGA |
| | | | GCCGAGGACACCGCCGTGTACTACTG |
| | | | CACAAGATACGGCGGCCTGGGCGCCA |
| | | | TGGACTACTGGGGCCAAGGCACCCTG |
| | | | GTGACCGTCAGCTCC |
| | SEQ ID NO: 67 | VL | GACATTCAGATGACACAGAGCCCTAG |
| | | DNA | CAGCCTGAGCGCTAGCGTGGGCGACA |
| | | | GAGTGACCATCACCTGCAGAGCTAGC |
| | | | GGCAACATCCACAACTACCTGACCTG |
| | | | GTATCAGCAGAAGCCCGGCAAGGCCC |
| | | | CCAAGCTGCTGATCTACAACGCCAAG |
| | | | ACCCTGGCCAGCGGCGTGCCTAGCAG |
| | | | ATTCAGCGGCAGCGGCAGCGGCACCG |
| | | | ACTTCACCCTGACCATCAGCAGCCTGC |
| | | | AGCCCGAGGACTTCGCCACCTACTACT |
| | | | GTCAGCACTTCTGGAGCGTGCCCTACA |
| | | | CCTTCGGCCAAGGCACCAAGCTGGAG |
| | | | ATCAAG |
| huBG219P- | SEQ ID NO: 24 | HCDR1 | DYIMS |
| E43 | | (Kabat) | |
| | SEQ ID NO: 25 | HCDR2 | TVSSGGDNTYYPDSVKG |
| | | (Kabat) | |
| | SEQ ID NO: 26 | HCDR3 | YGGLGAMDY |
| | | (Kabat) | |
| | SEQ ID NO: 27 | LCDR1 | RASGNIHNYLT |
| | | (Kabat) | |
| | SEQ ID NO: 65 | LCDR2 | NAKTLAS |
| | | (Kabat) | |
| | SEQ ID NO: 29 | LCDR3 | QHFWSVPYT |
| | | (Kabat) | |
| | SEQ ID NO: 61 | VH | EVQLLESGGGLVQPGGSLRLSCAASGFT |
| | | AA | FNDYIMSWVRQAPGKGLEWVATVSSGG |
| | | | DNTYYPDSVKGRFTISRDNSKNTLYLQM |
| | | | NSLRAEDTAVYYCTRYGGLGAMDYWG |
| | | | QGTLVTVSS |
| | SEQ ID NO: 68 | VL | DIQMTQSPSSLSASVGDRVTITCRASGNI |
| | | AA | HNYLTWYQQKPGKAPKLLIYNAKTLAS |
| | | | GVPSRFSGSGSGTDYTLTISSLQPEDFAT |
| | | | YYCQHFWSVPYTFGQGTKLEIK |
| | SEQ ID NO: 63 | VH | GAAGTGCAGCTGCTCGAGAGCGGGGG |
| | | DNA | CGGCCTCGTGCAACCTGGCGGGAGCC |
| | | | TGAGACTGAGCTGCGCCGCTAGCGGC |
| | | | TTCACCTTCAACGACTACATCATGAGC |
| | | | TGGGTGAGACAAGCCCCCGGCAAGGG |
| | | | CCTGGAGTGGGTGGCCACCGTGAGCA |
| | | | GCGGGGGCGACAACACCTACTACCCC |
| | | | GACAGCGTGAAGGGCAGATTCACCAT |
| | | | CAGCAGAGACAACAGCAAGAACACCC |
| | | | TGTACCTGCAGATGAACAGCCTGAGA |
| | | | GCCGAGGACACCGCCGTGTACTACTG |
| | | | CACAAGATACGGCGGCCTGGGCGCCA |
| | | | TGGACTACTGGGGCCAAGGCACCCTG |
| | | | GTGACCGTCAGCTCC |
| | SEQ ID NO: 69 | VL | GACATTCAGATGACACAGAGCCCTAG |
| | | DNA | CAGCCTGAGCGCTAGCGTGGGCGACA |
| | | | GAGTGACCATCACCTGCAGAGCTAGC |
| | | | GGCAACATCCACAACTACCTGACCTG |
| | | | GTATCAGCAGAAGCCCGGCAAGGCCC |
| | | | CCAAGCTGCTGATCTACAACGCCAAG |
| | | | ACCCTGGCCAGCGGCGTGCCTAGCAG |
| | | | ATTCAGCGGCAGCGGCAGCGGCACCG |
| | | | ACTACACCCTGACCATCAGCAGCCTGC |
| | | | AGCCCGAGGACTTCGCCACCTACTACT |
| | | | GTCAGCACTTCTGGAGCGTGCCCTACA |
| | | | CCTTCGGCCAAGGCACCAAGCTGGAG |
| | | | ATCAAG |

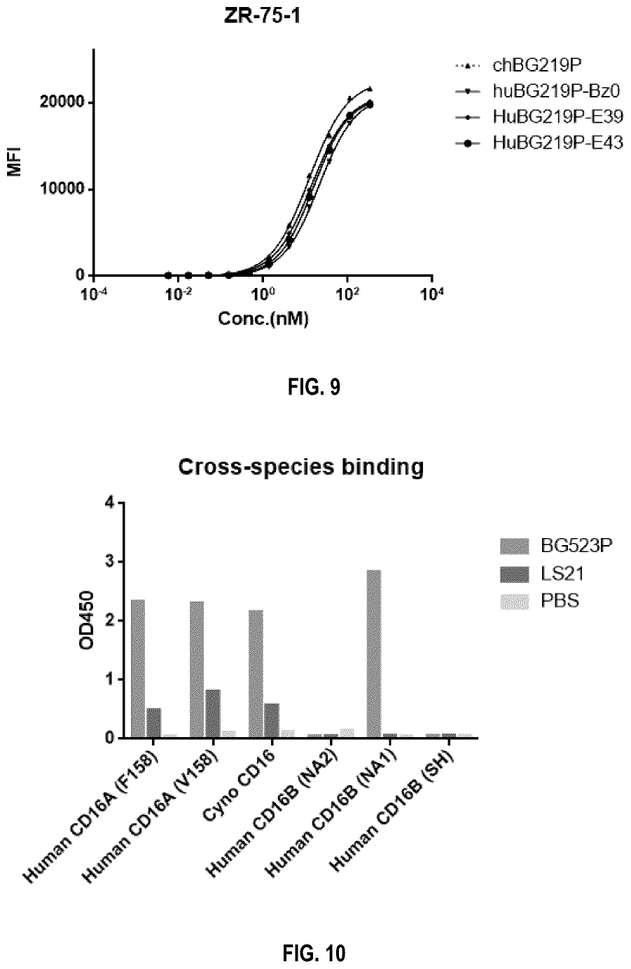

To evaluate the binding activity of anti-MUC1 antibodies to bind native MUC1 on live cells, ZR-75 cells were used for FACS-based binding assay. Live ZR-75 cells were seeded in 96-well plates and were incubated with a series of dilutions of chimeric or humanized BG29Ps. Goat anti-human IgG was used as a second antibody to detect antibody binding to the cell surface. EC₅₀ values for dose-dependent binding to human native MUC1 were determined by fitting the dose-response data to the four-parameter logistic model with GraphPad Prism. As shown in FIG. 9 and Table 9, the humanized BG219P antibodies huBG219P-Bz0, E39, and E43 retained comparable binding affinity to native MUC1 compared with chimeric BG219P.

TABLE 9
Comparison of chBG219P and huBG219P binding
affinities to native MUC1 by FACS

| Ab | ZR-75 binding EC₅₀ (nM) |
|---|---|
| chBG219P | 12.3 |
| huBG219P-bz0 | 20.9 |
| huBG219P-E39 | 14.0 |
| huBG219P-E43 | 15.7 |

### Example 8. Generation of Anti-CD16A VHH

### Human CD16A Recombinant Protein and Cell Lines for Immunization and Assay

A recombinant six-histidine-tagged extracellular domain (ECD) fragment of human CD16A protein (V158) (SEQ ID NO: 101)—referred to as human CD16A-His₆ (V158)—was pure based from a commercial source (Sino Biologics) and utilized as the antigen to immunize an alpaca. Recombinant six-histidine-tagged ECD fragments of human CD16A (F158) (SEQ ID NO: 102), human CD16B (NA11) (SEQ ID NO: 103), human CD16B (NA2) (SEQ ID NO: 104), human CD16B (SH) (SEQ ID NO: 105), cyno CD16 (SEQ ID NO: 106)—referred to as human CD16A-His₆ (F158), human CD16B-His₆ (NA11), human CD16B-His₆ (NA2), human CD16B-His₆, (SH), and cyno CD16-His₆ respectively—were purchased from a commercial source (Sino Biologics) and used for various in vitro assays.

To facilitate screening and detection, a DNA fragment for human CD16A (V158) ECD (AA 1-208 of SEQ ID NO: 101) was fused with a C-terminal human IgG1 mf Fc tag (SEQ ID NO: 107). mouse IgG2a Fc tag, or alpaca IgG2b Fc tag and subjected to transient expression in Expi293 cells (Thermofisher Scientific). Culture supernatant was harvested and clarified, and affinity purified with a Protein A column (Cytiva). The final products were buffer exchanged to DPBS by ultrafiltration/diafiltration (UF/DF), and stored at −80° C.

To evaluate the binding activity of the antibody to CD16A expressed on living cells, NK92mi (ATCC, CRL-2407) cells were engineered to over-express human CD16A (NK92mi/CD16A F158 and NK92mi/CD16A V158) by co-transducing expression plasmids containing CD16A (F158 or V158) and FcRγ cDNAs. NK92mi/CD16B (NA1) and NK92mi/CD16B (NA2) expressing cell lines were prepared similarly from CD16B (NA1) or CD16B (NA2) expression plasmids.

### Immunization and Screening

One alpaca was immunized with recombinant protein human CD16A-His₆ (V158) as the antigen by an external contract research organization and immune VHH phage library was constructed from isolated alpaca peripheral blood mononuclear cells (PBMC) (Pardon Els et al. (2014) Nature Protocols) after the third immunization. Phage display selection was carried out using standard protocols (Silacci et al., (2005) Proteomics, 5, 2340-50; Zhao et al., (2014) PLoS One, 9, e111339). In brief, 10 mg/ml of immobilized human CD16A-V158-AlpacaIgG2b in immunotube was utilized to enrich human CD16A(V158) specific binders in panning round 1 and 2. Immunotube was blocked with 5% milk powder (w/v) in PBS supplemented with 1% Tween 20 (MPBST) for 1 h. After washing with PBST (PBS buffer supplemented with 0.05% Tween 20), 1×10¹³ (round 1) or 2×10¹² (round 2) phages were initially depleted by human CD16B-His₆ (NA2) in MPBST for 1 hour and then incubated with the antigen for 1 hour. After washing with PBST, bound phages were eluted with 100 mM triethylamine (Sigma-Aldrich). Eluted phages were used to infect mid-log phase E. coli TG1 bacteria and plated onto 2×YT (Yeast Extract Tryptone)-agar plates supplemented with 2% glucose and 100 μg/mL ampicillin. After three rounds of selections, individual clones were picked up and phage-containing supernatants were prepared using standard protocols. Phage ELISA was used to screen for anti-human CD16A antibodies.

For phage ELISA, a Maxisorp immunoplate was coated with recombinant protein human CD16A-His₆ (V158) as antigen and blocked with 5% milk powder (w/v) in PBS buffer. Phage supernatant was blocked with MPBST for 30 min and added to wells of the ELISA plate for 1 hour. After washing with PBST, bound phage was detected using HRP-conjugated anti-M13 antibody (GE Healthcare) and 3,3′,5,5′-tetramethylbenzidine substrate (Cat.: 004201-56, eBioscience, USA).

Positive clones from phage ELISA were sequenced and recovered. Six anti-CD16A VHH variants were constructed by fusing their open reading frames with a C-terminal human IgG1 mf Fc (SEQ ID NO: 107) tag eukaryotic expression vector. The plasmids were transfected into ExpiCHO-s cells (Thermofisher Scientific) using MAX Titer protocol. The Fc-tagged VHH variants (VHH-Fc) were purified by MabSelect SuRe (Cytiva), followed by SPHP column (Cytiva). The final products were buffer exchanged to DPBS by UF/DF and stored at −80° C. for later use, including binding analysis.

For antigen ELISA, a Maxisorp immunoplate was coated with antigens (human CD16A (V158), human CD16A (F158), human CD16B (NA1), human CD16B (NA2), human CD16B (SH), or cyno CD16) and blocked with 3% BSA (w/v) in PBS buffer (blocking buffer). Monoclonal VHH-Fc antibodies were blocked with blocking buffer for 30 min and added to wells of the ELISA plate for 1 h. After washes with PBST, bound antibodies were detected using HRP-conjugated anti-human IgG antibody (Sigma, A0170) and 3,3′,5,5′-tetramethylbenzidine substrate (Cat.: 00-4201-56, eBioscience, USA).

For flow cytometry, NK92mi/CD16A V158 cells, NK92mi/CD16B(NA1) cells, and NK92mi/CD16B(NA2) cells (10⁵ cells/well) were incubated with various concentrations of IgG-like antibodies, followed by binding with Alexa Fluro-647-labeled anti-human IgG Fc antibody (Cat.: 409320, BioLegend, USA). Cell fluorescence was quantified using a flow cytometer (Guava easyCyte™ 8HT, Merck-Millipore, USA).

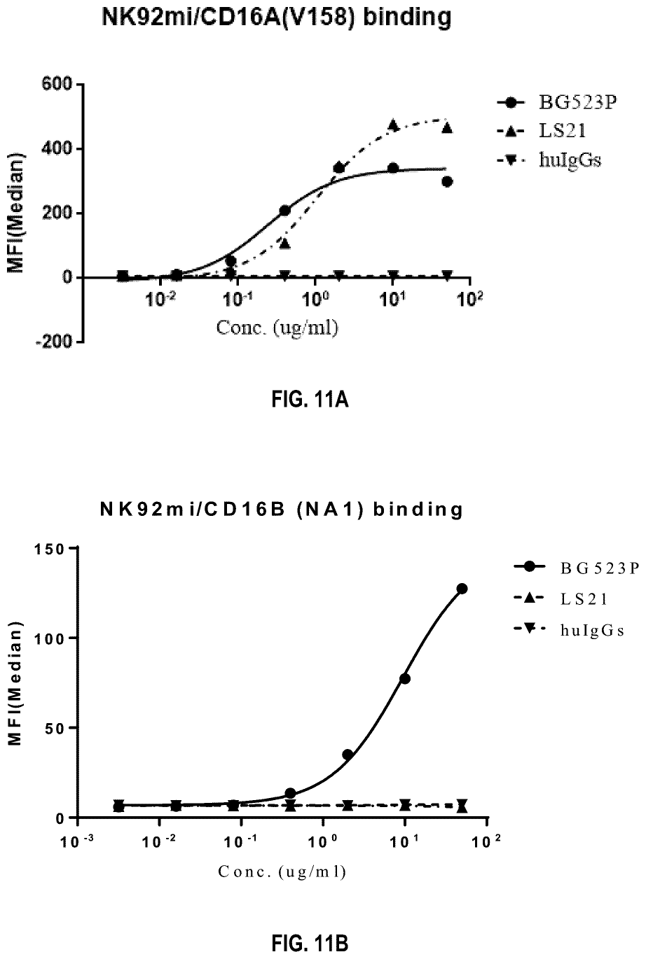

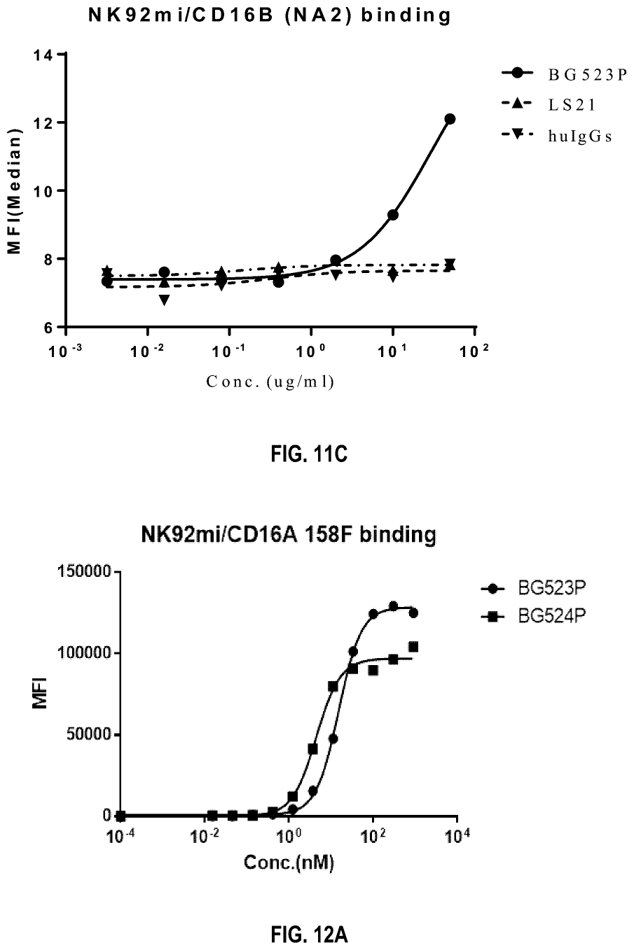

Following the procedures disclosed above, 44 positive clones were sequenced and recovered, and one representative positive anti-CD16A variant BG523P (VHH AA SEQ ID NO: 112, VHH DNA SEQ ID NO: 113) was obtained from six VHH-Fc fusion clones. The binding affinity to CD16A of BG523P was also confirmed by antigen ELISA. The ELISA and FACS analysis results of BG523P vs a positive control, LS21, are shown in Tables 10 to 12 and FIGS. 10 and 11A-11C. More specifically, data for FACS binding to NK92mi/CD16A cell lines of BG523P vs. a human CD16A specific binder LS21 as a positive control (SEQ ID NO: 108, Patent EP1888645B1) are given in Table 10, which shows BG523P has specificity for binding with NK92mi/CD16A cells. FIG. 10 illustrates that BG523P, compared to LS21, showed a higher binding with human CD16A, CD16B (NA1), and cyno CD16 at 1 mg/ml. FIG. 11A shows BG523P specifically binds with NK92mi/CD16A cells. FIGS. 11B and 11C show BG523P exhibits weak binding with NK92mi/CD16B at high concentrations. Although BG523P exhibits a slight binding activity to CD16B (NA1) in an ELISA assay as well as in a FACS assay (FIG. 10. Table 11), its binding affinity was significantly reduced (the calculated EC₅₀ value was decreased about 40-fold in comparison to that of CD16A binding in a FACS assay). This result suggests that BG523P could selectively bind to CD16A over CD16B. FIG. 10 also shows that BG523P barely binds to CD16B SH allotype. Considering that the predominant variants for human CD16B are NA1 and NA2 allotypes and the frequency for the SH allotype is rare and reported to be less than 0.05 in Caucasians. binding properties toward CD16B SH allotype were not further characterized.

TABLE 10
FACS based binding of VHH to NK92mi/CD16A cell lines

| | BG523P | LS21 | Human IgG |
|---|---|---|---|
| EMAX | 342.0 | 504.8 | 7.55 |
| EC₅₀ (μg/mL) | 0.242 | 1.041 | 0.01122 |

TABLE 11
FACS based binding of VHH to NK92mi/CD16B (NA1) cell lines

| | BG523P | LS21 | Human IgG |
|---|---|---|---|
| EMAX | 149.5 | ~−2874 | 7.456 |
| EC₅₀ (μg/mL) | 9.518 | ~155157 | 10.30 |

TABLE 12
FACS based binding of VHH to NK92mi/CD16B (NA2) cell lines

| | BG523P | LS21 | Human IgG |
|---|---|---|---|
| EMAX | 14.82 | 7.823 | 7.655 |
| EC₅₀ (μg/mL) | 28.90 | 0.1363 | 0.3139 |

### Example 9. Humanization of the Anti-Human CD16A VHH BG523P

For humanization of BG523P, human germline IgG genes were searched for sequences that share high degrees of homology to the cDNA sequences of BG523P variable regions by blasting the human immunoglobulin gene database in IMGT (http://www.imgt.org/IMGT_vquest/share/textes/index.html) and NCBI (http://www.ncbi.nlm.nih.gov/igblast/) websites. The human IGVH genes that are present in human antibody repertoires with high frequencies (Glanville 2009 PNAS 106:20216-20221) and are highly homologous to BG523P were selected as the templates for humanization.

Humanization was carried out by CDR-grafting (Methods in Molecular Biology, Vol 248: Antibody Engineering, Methods and Protocols. Humana Press) and the humanized VHH variants were engineered as VHH-Fc using an in-house developed expression vector for later binding and biophysical stability analysis, etc. In the initial round of humanization, mutations from camelid to human amino acid residues in framework regions were guided by the simulated 3D structure, and the camelid framework residues of structural importance for maintaining the canonical structures of CDRs were retained in the first versions of humanized BG523P. Among the many variants, BG524P is a preferred humanized VHH with the most retained camelid residues. Specifically, HCDR1 (SEQ ID NO: 109) and HCDR3 (SEQ ID NO: 111) of BG523P were grafted into the framework of human germline variable gene IGVH3-7 with 5 camelid framework residues (F37, R45, V78, P84, and A94 by Kabat numbering) retained, while one mutation was introduced in HCDR2 to remove a potential isomerization site. The sequences of BG524P are provided as SEQ ID NOs: 109, 114, 111, and 115-116 in Table 23.

Humanized BG523P variants were fused to the N-terminal of Fc as VHH-Fc format using in-house developed expression vectors that contain Fc region of a human IgG1 variant (SEQ ID NO: 107), with easy adapting sub-cloning sites. Expression and preparation of humanized BG523P VHH-Fc antibodies were achieved by transfection of the constructs into ExpiCHO-s cells and by purification using a protein A column. The purified VHH-Fc antibodies were concentrated to 0.5-5 mg/mL in PBS and stored in aliquots in a −80° C. freezer.

For affinity determination, VHH-Fc antibodies were captured by anti-human Fc surface and used in an affinity assay based on surface plasmon resonance (SPR) technology. The results of SPR-determined binding profiles of anti-CD16A VHH are summarized in Table 13. BG524P showed slightly improved binding affinities to CD16A 158V and CD16A 158F with dissociation constants at 0.08 nM and 0.08 nM, respectively, compared with those of BG523P. Meanwhile, BG524P kept the selectivity to CD16A over CD16B as characterized by SPR.

TABLE 13
Comparison of anti-CD16A VHHs binding affinities
to human CD16A by SPR

| VHH | | BG523P | BG524P |
|---|---|---|---|
| CD16A 158V | kₒₙ (M⁻¹s⁻¹) | 2.76E+05 | 1.35E+06 |
| | k_{off} (s⁻¹) | 1.13E−04 | 1.10E−04 |
| | K_{D} (nM) | 4.08E−10 | 8.16E−11 |
| CD16A 158F | kₒₙ (M⁻¹s⁻¹) | 3.19E+05 | 1.44E+06 |
| | k_{off} (s⁻¹) | 1.28E−04 | 1.19E−04 |
| | K_{D} (nM) | 4.02E−10 | 8.25E−11 |
| CD16B NA1 | K_{D} (nM) | 1.70E−06 | 9.42E−07 |
| CD16B NA2 | K_{D} (nM) | 4.26E−06 | 1.05E−06 |

### Using CD16A 158F Over-Expressing Cell Line NK92mi/CD16A F158 to Evaluate the Ability of Anti-CD16A VHH-Fc Antibodies to Bind Native CD16A on Live Cells.

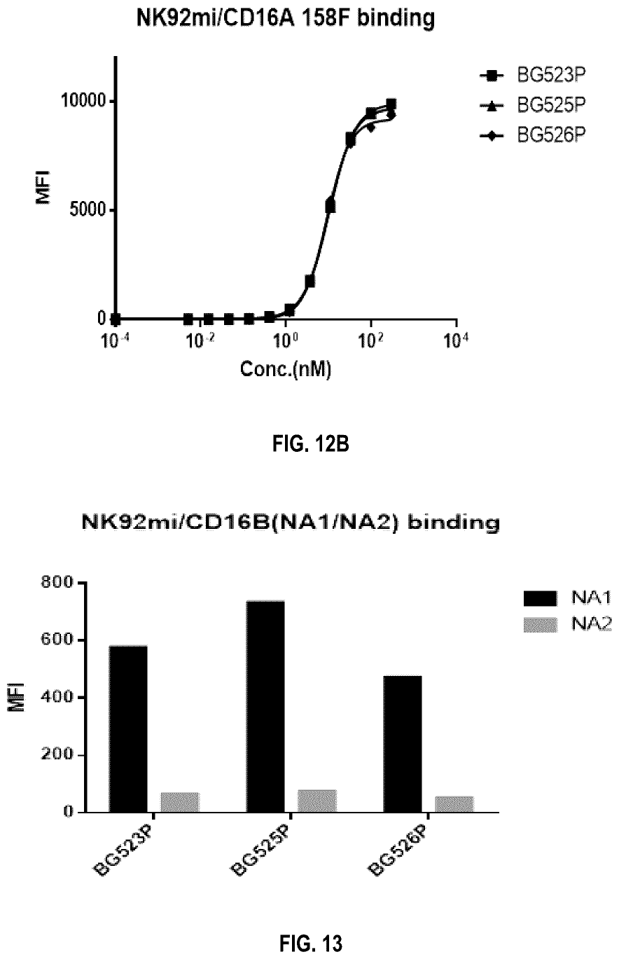

Live NK92mi/CD16A 158F cells were seeded in 96-well plates and were incubated with a series of dilutions of anti-CD16A VHH-Fc. Goat anti-human IgG was used as a second antibody to detect antibody binding to the cell surface. EC₅₀ values for dose-dependent binding to human native CD16A were determined by fitting the dose-response data to the four-parameter logistic model with GraphPad Prism. As shown in FIG. 12A and Table 14, BG524P showed improved binding affinity to native CD16A 158F but showed decreased Emax.

TABLE 14
Comparison of anti-CD16A VHHs binding affinities
to human CD16A by FACS

| VHH | BG523P | BG524P |
|---|---|---|
| NK92mi/CD16A 158F binding EC₅₀ (nM) | 15.06 | 4.42 |
| NK92mi/CD16A 158F binding Emax (MFI) | 128500 | 96964 |

To determine if the humanized BG523P kept the optimal biophysical stability of BG523P, the melting temperature (Tm) and aggregation temperature (Tagg) of BG524P were determined and compared with those of BG523P. BG524P showed both inferior Tm and Tagg compared with those of BG523P (Table 15).

Melting temperature (Tm) was determined using a high throughput MicroCal™ VP-Capillary DSC (Malvern Instruments, Northampton, MA). Thermograms for each protein (350 μL at 0.5 mg/mL) were obtained from 20° C. to 100° C. using a scan rate of 60° C./hr. Thermograms of the buffer alone were subtracted from each protein sample. Obtained results show the values for the midpoint of transition temperatures (Tm) and the calorimetric enthalpy (Ai) of the sample.

The aggregation temperature Tagg (° C.) is representative of the colloidal stability of the samples and was obtained by monitoring the onset of aggregation by SLS266 using UNCLE™ (Unchained lab, Pleasanton, CA). Samples were loaded into Uni, and subjected to a temperature ramping from 15° C. to 95° C. The back-reflection optics cannot detect near UV light scattering by protein aggregates, and thus only non-scattered light reaches the detector. The reduction of back reflected light is therefore a direct measure for aggregation in the sample.

TABLE 15
Comparison of thermal stabilities and colloidal
stabilities of anti-CD16A VHHs

| VHH-Fc | Tm (° C.) | Tagg (° C.) |
|---|---|---|
| BG523P | 69.1 | 68.0 |
| BG524P | 59.7 | 54.5 |

BG524P was further engineered by introducing mutations in CDRs and back mutations in framework regions to improve biophysical properties, remove PTM sites, and recover binding Emax to native CD16A for therapeutic use in humans.

Taken together, the well-engineered versions of humanized monoclonal antibodies, BG525P (SEQ ID NOs: 109-111 and 117-118) and BG526P (SEQ ID NOs: 109, 114, 111, and 119-120), were derived from the mutation process described above, and both retained binding affinity to CD16A, selectivity over CD161B, and optimal biophysical stability of the parental clone, as characterized in detail (Tables 16 to 18 and FIG. 12B).

TABLE 16
Comparison of anti-CD16A VHHs binding affinities
to human CD16A by SPR

| VHH | | BG523P | BG525P | BG526P |
|---|---|---|---|---|
| CD16A 158V | kₒₙ (M⁻¹s⁻¹) | 2.80E+05 | 2.55E+05 | 2.89E+05 |
| | k_{off} (s⁻¹) | 6.22E−05 | 1.03E−04 | 1.33E−04 |
| | K_{D} (nM) | 2.22E−10 | 4.04E−10 | 4.25E−10 |
| CD16A 158F | kₒₙ (M⁻¹s⁻¹) | 2.24E+05 | 2.10E+05 | 2.30E+05 |
| | k_{off} (s⁻¹) | 7.88E−05 | 1.20E−04 | 1.48E−04 |
| | K_{D} (nM) | 3.53E−10 | 5.75E−10 | 6.40E−10 |
| CD16B NA1 | K_{D} (nM) | 1.76E−06 | 2.31E−06 | 2.74E−06 |
| CD16B NA2 | K_{D} (nM) | 4.95E−06 | 5.78E−06 | 7.64E−06 |

TABLE 17
Comparison of anti-CD16A VHHs binding affinities to human CD16A by FACS

| VHH | BG523P | BG525P | BG526P |
|---|---|---|---|
| NK92mi/CD16A 158F binding EC₅₀ (nM) | 10.6 | 10.3 | 9.1 |
| NK92mi/CD16A 158F binding EMAX(MFI) | 9915 | 9708 | 9197 |

TABLE 18
Comparison of thermal stabilities and colloidal stabilities
of anti-CD16A VHHs

| VHH-Fc | BG523P | BG525P | BG526P |
|---|---|---|---|
| Tm (° C.) | 69.1 | 69.1 | 68.1 |
| Tagg (° C.) | 68 | 68.1 | 65.2 |

### Example 10. Native CD16B Binding of Anti-CD16A VHHs

To evaluate the ability of anti-CD16A VHH to bind native CD16B on live cells. NK92mi cells were engineered to over-express human CD16B NA or NA2. Live NK92mi/CD16B cells were seeded in 96-well plates, and were incubated with 300 nM of anti-CD16A VHH-Fc. Goat anti-human IgG was used as a second antibody to detect anti-CD16A VHH-Fc binding to the cell surface. The binding signals of humanized VHH-Fc to CD16B were comparable with or lower than those of the parental clone as shown in FIG. 13 and Table 19, and were significantly lower than corresponding binding signals of those to CD16A (Table 17, FIG. 12A-12B).

TABLE 19
Comparison of anti-CD16A VHHs with parental clone
on the binding to human CD16B by FACS

| | NK92mi/CD16B NA1 | NK92mi/CD16B NA2 |
|---|---|---|
| | Binding signal | Binding signal |
| VHH | at 300 nM (MFI) | at 300 nM (MFI) |
| BG523P | 577 | 65 |
| BG525P | 733 | 75 |
| BG526P | 473 | 52 |

### Example 11. Human IgG Competition on VHH Binding to Native CD16A

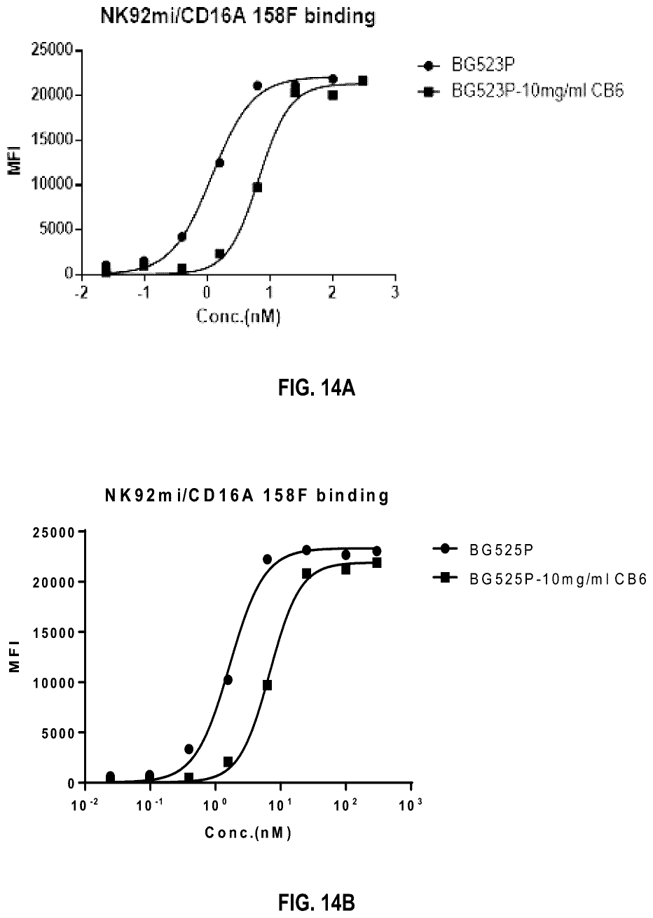

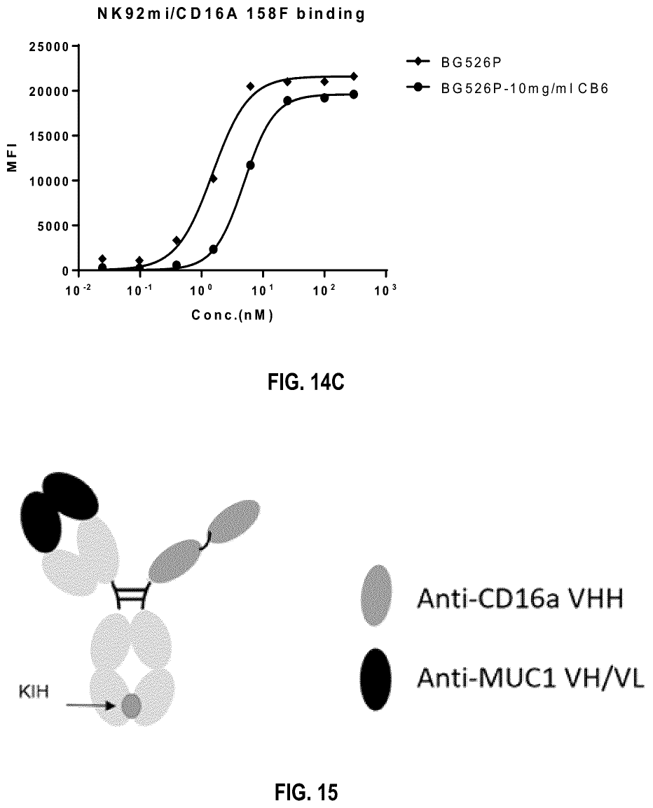

To evaluate the effect of human IgG competition on the ability of anti-CD6A VHH-Fc to bind native CD16A on live cells, a FACS-based assay was performed with or without the presence of human IgG. Live NK92mi/CD16A cells were seeded in 96-well plates, and were incubated at 37° C. with a series of dilutions of biotinylated anti-CD16A VHH-Fc alone or together with 10 mg/ml of a human IgG1 antibody C1B6 (anti-SARS-Covid19 antibody) (SEQ ID NO: 121-122). Streptavidin-AF647 was used as a second antibody to detect biotinylated anti-CD16A VHH-Fc binding to the cell surface. EC₅₀ values for dose-dependent binding to human native CD16A were determined by fitting the dose-response data to the four-parameter logistic model with GraphPad Prism. As shown in FIGS. 14A-14C and Table 20, the binding of BG525P and BG526P to CD16A 158F were similarly affected by the presence of human IgG compared with that of parental BG523P.

TABLE 20
Human IgG competition on BG523P derived anti-CD16A
VHHs binding affinities to human CD16A 158F by FACS

| VHH | | BG523P | BG525P | BG526P |
|---|---|---|---|---|
| Without | NK92mi/CD16A 158F | 1.19 | 1.65 | 1.52 |
| human IgG1 | binding EC₅₀(nM) | | | |
| (CB6) | NK92mi/CD16A 158F | 22122 | 23336 | 21614 |
| | binding EMAX(MFI) | | | |
| With | NK92mi/CD16A 158F | 6.57 | 6.83 | 4.95 |
| 10 mg/ml | binding EC₅₀(nM) | | | |
| human IgG1 | NK92mi/CD16A 158F | 21335 | 21914 | 19609 |
| (CB6) | binding EMAX(MFI) | | | |

### Example 12. Binding Affinity of Humanized CD16A to Cyno CD16 by SPR

For affinity determination, VHH-Fcs were captured by anti-human Fc surface, and used in the affinity assay based on surface plasmon resonance (SPR) technology. The results of SPR-determined binding profiles of anti-CD16A VHH-Fc are summarized in Table 21. Humanized anti-CD16A VHH-Fcs retained cross-reactivity to cyno CD16.

TABLE 21
Binding affinities of anti-CD16A VHHs to cyno CD16 by SPR

| | Cyno CD16 | | |
|---|---|---|---|
| VHH | kₒₙ (M⁻¹s⁻¹) | k_{off} (s⁻¹) | K_{D} (nM) |
| BG523P | 2.80E+05 | 8.09E−05 | 2.89E−10 |
| BG525P | 2.67E+05 | 1.12E−04 | 4.18E−10 |
| BG526P | 3.01E+05 | 1.47E−04 | 4.90E−10 |

TABLE 22
CD16A and CD16B Sequence List

| Name | SEQ ID | SEQ |
|---|---|---|
| Human | SEQ ID NO: 101 | MWQLLLPTALLLLVSAGMRTEDLPKAVVFLEPQWYRVLEKDS |
| CD16A | | VTLKCQGAYSPEDNSTQWFHNESLISSQASSYFIDAATVDDSG |
| (V158) | | EYRCQTNLSTLSDPVQLEVHIGWLLLQAPRWVFKEEDPIHLRC |
| | | HSWKNTALHKVTYLQNGKGRKYFHHNSDFYIPKATLKDSGS |
| | | YFCRGLVGSKNVSSETVNITITQGLAVSTISSFFPPGYQVSFCLV |
| | | MVLLFAVDTGLYFSVKTNIRSSTRDWKDHKFKWRKDPQDK |
| Human | SEQ ID NO: 102 | MWQLLLPTALLLLVSAGMRTEDLPKAVVFLEPQWYRVLEKDS |
| CD16A | | VTLKCQGAYSPEDNSTQWFHNESLISSQASSYFIDAATVDDSG |
| (F158) | | EYRCQTNLSTLSDPVQLEVHIGWLLLQAPRWVFKEEDPIHLRC |
| | | HSWKNTALHKVTYLQNGKGRKYFHHNSDFYIPKATLKDSGS |
| | | YFCRGLFGSKNVSSETVNITITQGLAVSTISSFFPPGYQVSFCLV |
| | | MVLLFAVDTGLYFSVKTNIRSSTRDWKDHKFKWRKDPQDK |
| Human | SEQ ID NO: 103 | MWQLLLPTALLLLVSAGMRTEDLPKAVVFLEPQWYRVLEKDS |
| CD16B | | VTLKCQGAYSPEDNSTQWFHNENLISSQASSYFIDAATVDDSG |
| (NA1) | | EYRCQTNLSTLSDPVQLEVHVGWLLLQAPRWVFKEEDPIHLR |
| | | CHSWKNTALHKVTYLQNGKDRKYFHHNSDFHIPKATLKDSG |
| | | SYFCRGLVGSKNVSSETVNITITQGLAVSTISSFSPPGYQVSFCL |
| | | VMVLLFAVDTGLYFSVKTNI |
| Human | SEQ ID NO: 104 | MWQLLLPTALLLLVSAGMRTEDLPKAVVFLEPQWYSVLEKDS |
| CD16B | | VTLKCQGAYSPEDNSTQWFHNESLISSQASSYFIDAATVNDSG |
| (NA2) | | EYRCQTNLSTLSDPVQLEVHIGWLLLQAPRWVFKEEDPIHLRC |
| | | HSWKNTALHKVTYLQNGKDRKYFHHNSDFHIPKATLKDSGS |
| | | YFCRGLVGSKNVSSETVNITITQGLAVSTISSFSPPGYQVSFCLV |
| | | MVLLFAVDTGLYFSVKTNI |
| Human | SEQ ID NO: 105 | MWQLLLPTALLLLVSAGMRTEDLPKAVVFLEPQWYSVLEKDS |
| CD16B | | VTLKCQGAYSPEDNSTQWFHNENLISSQASSYFIDAATVNDSG |
| (SH) | | EYRCQTNLSTLSDPVQLEVHIGWLLLQAPRWVFKEEDPIHLRC |
| | | HSWKNTALHKVTYLQNGKDRKYFHHNSDFHIPKATLKDSGS |
| | | YFCRGLVGSKNVSSETVNITITQGLAVSTISSFSPPGYQVSFCLV |
| | | MVLLFAVDTGLYFSVKTNI |
| Cyno | SEQ ID NO: 106 | MWQLLLPTALLLLVSAGMRAEDLPKAVVFLEPQWYRVLEKD |
| CD16 | | RVTLKCQGAYSPEDNSTRWFHNESLISSQTSSYFIAAARVNNS |
| | | GEYRCQTSLSTLSDPVQLEVHIGWLLLQAPRWVFKEEESIHLR |
| | | CHSWKNTLLHKVTYLQNGKGRKYFHQNSDFYIPKATLKDSGS |
| | | YFCRGLIGSKNVSSETVNITITQDLAVSSISSFFPPGYQVSFCLV |
| | | MVLLFAVDTGLYFSMKKSIPSSTRDWEDHKFKWSKDPQDK |
| human | SEQ ID NO: 107 | EPKSSDKTHTCPPCPAPPAAGPSVFLFPPKPKDTLMISRTPEVTC |
| IgGI mf | | VVVDVSHEDPEVKFNWYVDGVEVHNAKTKPREEQYNSTYRV |
| Fc | | VSVLTVLHQDWLNGKEYKCKVSNKALAAPIEKTISKAKGQPR |
| | | EPQVYTLPPSREEMTKNQVSLTCLVKGFYPSDIAVEWESNGQP |
| | | ENNYKTTPPVLDSDGSFFLYSKLTVDKSRWQQGNVFSCSVMH |
| | | EALHNHYTQKSLSLSPGK |
| LS21 | SEQ ID NO: 108 | EVQLVQSGAEVKKPGESLKVSCKASGYTFTSYYMHWVRQAP |
| | | GQGLEWMGIINPSGGSTSYAQKFQGRVTMTRDTSTSTVYMEL |
| | | SSLRSEDTAVYYCARGSAYYYDFADYWGQGTLVTVSSGGGG |
| | | SGGGGSGGGGSQPVLTQPSSVSVAPGQTATISCGGHNIGSKNV |
| | | HWYQQRPGQSPVLVIYQDNKRPSGIPERFSGSNSGNTATLTISG |
| | | TQAMDEADYYCQVWDNYSVLFGGGTKLTVL |

TABLE 23
Anti-CD16A VHH sequence list

| Antibody | SEQ ID NO | Note | SEQUENCE |
|---|---|---|---|
| BG523P | SEQ ID NO: 109 | CDR1 | SYAVG |
| | | (Kabat) | |
| | SEQ ID NO: 110 | CDR2 | AIRRSDGTTDYVDSVKG |
| | | (Kabat) | |
| | SEQ ID NO: 111 | CDR3 | SDSDYEPLGDY |
| | | (Kabat) | |
| | SEQ ID NO: 112 | VHH AA | QVQLVESGGGVVQPGGSLTLSCEASGRTFGS |
| | | | YAVGWFRQALGRGREFVAAIRRSDGTTDYV |
| | | | DSVKGRFTISRDNAKNTVYLQMNSLKPEDT |
| | | | AVYYCAASDSDYEPLGDYWGQGTQVTVSS |
| | SEQ ID NO: 113 | VHH DNA | CAGGTGCAGCTCGTGGAGTCTGGGGGCGG |
| | | | CGTGGTGCAGCCTGGGGGGTCTCTGACAC |
| | | | TCTCCTGTGAAGCCTCTGGACGCACCTTCG |
| | | | GCAGCTATGCCGTGGGCTGGTTCCGCCAG |
| | | | GCTCTTGGGAGGGGGCGTGAGTTTGTAGC |
| | | | AGCTATTAGGCGGAGTGATGGTACTACAGA |
| | | | CTATGTAGACTCCGTGAAGGGCCGATTCAC |
| | | | CATCTCCAGAGACAACGCCAAGAACACGG |
| | | | TGTATCTGCAAATGAACAGCCTGAAACCTG |
| | | | AGGACACGGCCGTTTATTACTGTGCAGCAT |
| | | | CGGATAGCGACTATGAACCCTTAGGGGACT |
| | | | ACTGGGGCCAGGGGACCCAGGTCACCGTC |
| | | | TCCTCA |
| BG524P | SEQ ID NO: 109 | CDR1 | SYAVG |
| | | (Kabat) | |
| | SEQ ID NO: 114 | CDR2 | AIRRSDATTDYVDSVKG |
| | | (Kabat) | |
| | SEQ ID NO: 111 | CDR3 | SDSDYEPLGDY |
| | | (Kabat) | |
| | SEQ ID NO: 115 | VHH AA | EVQLVESGGGLVQPGGSLRLSCAASGRTFGS |
| | | | YAVGWFRQAPGKGREWVAAIRRSDATTDYV |
| | | | DSVKGRFTISRDNAKNSVYLQMNSLRPEDT |
| | | | AVYYCAASDSDYEPLGDYWGQGTLVTVSS |
| | SEQ ID NO: 116 | VHH DNA | GAGGTGCAGCTCGTGGAATCCGGGGGGGG |
| | | | GCTGGTGCAACCCGGCGGGAGCCTGAGAC |
| | | | TGAGCTGCGCCGCTAGCGGCAGAACCTTC |
| | | | GGCAGCTACGCCGTGGGCTGGTTCAGACA |
| | | | AGCCCCCGGCAAGGGCAGAGAGTGGGTG |
| | | | GCCGCCATCAGAAGAAGCGACGCCACCAC |
| | | | CGACTACGTGGACAGCGTGAAGGGCAGAT |
| | | | TCACCATCAGCAGAGACAACGCCAAGAAC |
| | | | AGCGTGTACCTGCAGATGAACAGCCTGAG |
| | | | ACCCGAGGACACCGCCGTGTACTACTGCG |
| | | | CCGCCTCCGACAGCGACTACGAGCCTCTG |
| | | | GGCGATTACTGGGGCCAAGGCACCCTGGT |
| | | | GACCGTGAGCAGC |
| BG525P | SEQ ID NO: 109 | CDR1 | SYAVG |
| | | (Kabat) | |
| | SEQ ID NO: 110 | CDR2 | AIRRSDGTTDYVDSVKG |
| | | (Kabat) | |
| | SEQ ID NO: 111 | CDR3 | SDSDYEPLGDY |
| | | (Kabat) | |
| | SEQ ID NO: 117 | VHH AA | EVQLVESGGGLVQPGGSLRLSCAASGRTFGS |
| | | | YAVGWFRQAPGRGREFVAAIRRSDGTTDYV |
| | | | DSVKGRFTISRDNAKNTVYLQMNSLKPEDT |
| | | | AVYYCAASDSDYEPLGDYWGQGTLVTVSS |
| | SEQ ID NO: 118 | VHH DNA | GAGGTGCAGCTCGTGGAATCCGGGGGCGG |
| | | | GCTGGTGCAACCCGGCGGGAGCCTGAGAC |
| | | | TGAGCTGCGCCGCTAGCGGCAGAACCTTC |
| | | | GGCAGCTACGCCGTGGGCTGGTTCAGACA |
| | | | AGCCCCCGGCAGAGGCAGAGAGTTCGTGG |
| | | | CCGCCATCAGAAGAAGCGACGGCACCACC |
| | | | GACTACGTGGACAGCGTGAAGGGCAGATT |
| | | | CACCATCAGCAGAGACAACGCCAAGAACA |
| | | | CCGTGTACCTGCAGATGAACAGCCTGAAG |
| | | | CCCGAGGACACCGCCGTGTACTACTGCGC |
| | | | CGCCTCCGACAGCGACTACGAGCCTCTGG |
| | | | GCGATTACTGGGGCCAAGGCACCCTGGTG |
| | | | ACCGTGAGCAGC |
| BG526P | SEQ ID NO: 109 | CDR1 | SYAVG |
| | | (Kabat) | |
| | SEQ ID NO: 114 | CDR2 | AIRRSDATTDYVDSVKG |
| | | (Kabat) | |
| | SEQ ID NO: 111 | CDR3 | SDSDYEPLGDY |
| | | (Kabat) | |
| | SEQ ID NO: 119 | VHH AA | EVQLVESGGGLVQPGGSLRLSCAASGRTFGS |
| | | | YAVGWFRQAPGKGREFVAAIRRSDATTDYV |
| | | | DSVKGRFTISRDNAKNTVYLQMNSLKPEDT |
| | | | AVYYCAASDSDYEPLGDYWGQGTLVTVSS |
| | SEQ ID NO: 120 | VHH DNA | GAGGTGCAGCTCGTGGAATCCGGGGGCGG |
| | | | GCTGGTGCAACCCGGCGGGAGCCTGAGAC |
| | | | TGAGCTGCGCCGCTAGCGGCAGAACCTTC |
| | | | GGCAGCTACGCCGTGGGCTGGTTCAGACA |
| | | | AGCCCCCGGCAAGGGCAGAGAGTTCGTGG |
| | | | CCGCCATCAGAAGAAGCGACGCCACCACC |
| | | | GACTACGTGGACAGCGTGAAGGGCAGATT |
| | | | CACCATCAGCAGAGACAACGCCAAGAACA |
| | | | CCGTGTACCTGCAGATGAACAGCCTGAAG |
| | | | CCCGAGGACACCGCCGTGTACTACTGCGC |
| | | | CGCCTCCGACAGCGACTACGAGCCTCTGG |
| | | | GCGATTACTGGGGCCAAGGCACCCTGGTG |
| | | | ACCGTGAGCAGC |
| CB6 | SEQ ID NO: 121 | Heavy | EVQLVESGGGLVQPGGSLRLSCAASGFTVSS |
| | | chain | NYMSWVRQAPGKGLEWVSVIYSGGSTFYA |
| | | | DSVKGRFTISRDNSMNTLFLQMNSLRAEDT |
| | | | AVYYCARVLPMYGDYLDYWGQGTLVTVSS |
| | | | ASTKGPSVFPLAPSSKSTSGGTAALGCLVKD |
| | | | YFPEPVTVSWNSGALTSGVHTFPAVLQSSGL |
| | | | YSLSSVVTVPSSSLGTQTYICNVNHKPSNTK |
| | | | VDKRVEPKSCDKTHTCPPCPAPELLGGPSVF |
| | | | LFPPKPKDTLMISRTPEVTCVVVDVSHEDPE |
| | | | VKFNWYVDGVEVHNAKTKPREEQYNSTYR |
| | | | VVSVLTVLHQDWLNGKEYKCKVSNKALPA |
| | | | PIEKTISKAKGQPREPQVYTLPPSRDELTKNQ |
| | | | VSLTCLVKGFYPSDIAVEWESNGQPENNYKT |
| | | | TPPVLDSDGSFFLYSKLTVDKSRWQQGNVFS |
| | | | CSVMHEALHNHYTQKSLSLSPGK |
| | SEQ ID NO: 122 | Light chain | DIVMTQSPSSLSASVGDRVTITCRASQSISRY |
| | | | LNWYQQKPGKAPKLLIYAASSLQSGVPSRFS |
| | | | GSGSGTDFTLTISSLQPEDFATYYCQQSYSTP |
| | | | PEYTFGQGTKLEIKRTVAAPSVFIFPPSDEQL |
| | | | KSGTASVVCLLNNFYPREAKVQWKVDNAL |
| | | | QSGNSQESVTEQDSKDSTYSLSSTLTLSKAD |
| | | | YEKHKVYACEVTHQGLSSPVTKSFNRGEC |

TABLE 24
Amino acid and DNA sequences of MUC1xCD16A multi-specific antibody

| Antibody | SEQ ID NO | | SEQUENCE |
|---|---|---|---|
| BG526P | SEQ ID NO: | CDR1 (Kabat) | SYAVG |
| | 109 | | |
| | SEQ ID NO: | CDR2(Kabat) | AIRRSDATTDYVDSVKG |
| | 114 | | |
| | SEQ ID NO: | CDR3(Kabat) | SDSDYEPLGDY |
| | 111 | | |
| | SEQ ID NO: | VHH AA | EVQLVESGGGLVQPGGSLRLSCAASGRT |
| | 119 | | FGSYAVGWFRQAPGKGREFVAAIRRSDA |
| | | | TTDYVDSVKGRFTISRDNAKNTVYLQM |
| | | | NSLKPEDTAVYYCAASDSDYEPLGDYW |
| | | | GQGTLVTVSS |
| MUC1 | SEQ ID NO: 24 | CDRH1(Kabat) | DYIMS |
| huBG219P- | SEQ ID NO: 25 | CDRH2(Kabat) | TVSSGGDNTYYPDSVKG |
| E39 | SEQ ID NO: 26 | CDRH3(Kabat) | YGGLGAMDY |
| | SEQ ID NO: 27 | CDRL1(Kabat) | RASGNIHNYLT |
| | SEQ ID NO: 65 | CDRL2(Kabat) | NAKTLAS |
| | SEQ ID NO: 29 | CDRL3(Kabat) | QHFWSVPYT |
| | SEQ ID NO: 61 | VH AA | EVQLLESGGGLVQPGGSLRLSCAASGFT |
| | | | FNDYIMSWVRQAPGKGLEWVATVSSGG |
| | | | DNTYYPDSVKGRFTISRDNSKNTLYLQ |
| | | | MNSLRAEDTAVYYCTRYGGLGAMDYW |
| | | | GQGTLVTVSS |
| | SEQ ID NO: 66 | VL AA | DIQMTQSPSSLSASVGDRVTITCRASGNI |
| | | | HNYLTWYQQKPGKAPKLLIYNAKTLAS |
| | | | GVPSRFSGSGSGTDFTLTISSLQPEDFATY |
| | | | YCQHFWSVPYTFGQGTKLEIK |
| BG1222P | SEQ ID NO: | Chain 1 AA | EVQLVESGGGLVQPGGSLRLSCAASGRT |
| | 143 | (BG526P) | FGSYAVGWFRQAPGKGREFVAAIRRSDA |
| | | | TTDYVDSVKGRFTISRDNAKNTVYLQM |
| | | | NSLKPEDTAVYYCAASDSDYEPLGDYW |
| | | | GQGTLVTVSSGGGGSGGGGSEVOLVES |
| | | | GGGLVQPGGSLRLSCAASGRTFGSYAVG |
| | | | WFRQAPGKGREFVAAIRRSDATTDYVD |
| | | | SVKGRFTISRDNAKNTVYLQMNSLKPE |
| | | | DTAVYYCAASDSDYEPLGDYWGQGTLV |
| | | | TVSSEPKSSDKTHTCPPCPAPELLGGPSV |
| | | | FLFPPKPKDTLYITREPEVTCVVVDVSHE |
| | | | DPEVKFNWYVDGVEVHNAKTKPREEQ |
| | | | YNSTYRVVSVLTVLHQDWLNGKEYKC |
| | | | KVSNKALPAPIEKTISKAKGQPREPQVY |
| | | | TLPPSREEMTKNQVSLWCLVKGFYPSDI |
| | | | AVEWESNGQPENNYKTTPPVLDSDGSFF |
| | | | LYSKLTVDKSRWQQGNVFSCSVMHEAL |
| | | | HNHYTQKSLSLSPGK |
| | SEQ ID NO: | Chain 1 DNA | GAGGTGCAGCTCGTGGAATCCGGGGG |
| | 144 | (BG526P) | CGGGCTGGTGCAACCCGGCGGGAGCC |
| | | | TGAGACTGAGCTGCGCCGCTAGCGGC |
| | | | AGAACCTTCGGCAGCTACGCCGTGGG |
| | | | CTGGTTCAGACAAGCCCCCGGCAAGG |
| | | | GCAGAGAGTTCGTGGCCGCCATCAGA |
| | | | AGAAGCGACGCCACCACCGACTACGT |
| | | | GGACAGCGTGAAGGGCAGATTCACCA |
| | | | TCAGCAGAGACAACGCCAAGAACACC |
| | | | GTGTACCTGCAGATGAACAGCCTGAA |
| | | | GCCCGAGGACACCGCCGTGTACTACT |
| | | | GCGCCGCCTCCGACAGCGACTACGAG |
| | | | CCTCTGGGCGATTACTGGGGCCAAGG |
| | | | CACCCTGGTGACCGTGAGCAGCGGCG |
| | | | GTGGCGGCTCTGGTGGCGGCGGCTCT |
| | | | GAGGTGCAGCTCGTGGAATCCGGGGG |
| | | | CGGGCTGGTGCAACCCGGCGGGAGCC |
| | | | TGAGACTGAGCTGCGCCGCTAGCGGC |
| | | | AGAACCTTCGGCAGCTACGCCGTGGG |
| | | | CTGGTTCAGACAAGCCCCCGGCAAGG |
| | | | GCAGAGAGTTCGTGGCCGCCATCAGA |
| | | | AGAAGCGACGCCACCACCGACTACGT |
| | | | GGACAGCGTGAAGGGCAGATTCACCA |
| | | | TCAGCAGAGACAACGCCAAGAACACC |
| | | | GTGTACCTGCAGATGAACAGCCTGAA |
| | | | GCCCGAGGACACCGCCGTGTACTACT |
| | | | GCGCCGCCTCCGACAGCGACTACGAG |
| | | | CCTCTGGGCGATTACTGGGGCCAAGG |
| | | | CACCCTGGTGACCGTGAGCAGCGAGC |
| | | | CAAAGTCCTCTGACAAAACTCACACA |
| | | | TGCCCACCGTGCCCAGCACCTGAACTC |
| | | | CTGGGGGGACCGTCAGTCTTCCTCTTC |
| | | | CCCCCAAAACCCAAGGACACCCTCTA |
| | | | CATCACCCGGGAACCTGAGGTCACAT |
| | | | GCGTGGTGGTGGACGTGAGCCACGAA |
| | | | GACCCTGAGGTCAAGTTCAACTGGTA |
| | | | CGTGGACGGCGTGGAGGTGCATAATG |
| | | | CCAAGACAAAGCCGCGGGAGGAGCA |
| | | | GTACAACAGCACGTACCGTGTGGTCA |
| | | | GCGTCCTCACCGTCCTGCACCAGGACT |
| | | | GGCTGAATGGCAAGGAGTACAAGTGC |
| | | | AAGGTCTCCAACAAAGCCCTCCCAGC |
| | | | CCCCATCGAGAAAACCATCTCCAAAG |
| | | | CCAAAGGGCAGCCCCGAGAACCACAG |
| | | | GTGTACACCCTGCCCCCATCCCGGGA |
| | | | AGAGATGACCAAGAACCAGGTCAGCC |
| | | | TGTGGTGCCTGGTCAAAGGCTTCTATC |
| | | | CCAGCGACATCGCCGTGGAGTGGGAG |
| | | | AGCAATGGGCAGCCGGAGAACAACTA |
| | | | CAAGACCACGCCTCCCGTGCTGGACT |
| | | | CCGACGGCTCCTTCTTCCTCTACAGCA |
| | | | AGCTCACCGTGGACAAGAGCAGGTGG |
| | | | CAGCAGGGGAACGTCTTCTCATGCTCC |
| | | | GTGATGCATGAGGCTCTGCACAACCA |
| | | | CTACACGCAGAAGAGCCTCTCCCTGTC |
| | | | TCCGGGTAAATGA |
| | SEQ ID NO: | Chain 2 AA | EVQLLESGGGLVQPGGSLRLSCAASGFT |
| | 145 | (huBG219P-E39) | FNDYIMSWVRQAPGKGLEWVATVSSG |
| | | | GDNTYYPDSVKGRFTISRDNSKNTLYLQ |
| | | | MNSLRAEDTAVYYCTRYGGLGAMDY |
| | | | WGQGTLVTVSSASTKGPSVFPLAPSSKS |
| | | | TSGGTAALGCLVKDYFPEPVTVSWNSG |
| | | | ALTSGVHTFPAVLQSSGLYSLSSVVTVP |
| | | | SSSLGTQTYICNVNHKPSNTKVDKKVEP |
| | | | KSCDKTHTCPPCPAPELLGGPSVFLFPPK |
| | | | PKDTLYITREPEVTCVVVDVSHEDPEVK |
| | | | FNWYVDGVEVHNAKTKPREEQYNSTY |
| | | | RVVSVLTVLHQDWLNGKEYKCKVSNK |
| | | | ALPAPIEKTISKAKGQPREPQVYTLPPSR |
| | | | EEMTKNQVSLSCAVKGFYPSDIAVEWE |
| | | | SNGQPENNYKTTPPVLDSDGSFFLVSKL |
| | | | TVDKSRWQQGNVFSCSVMHEALHNHY |
| | | | TQKSLSLSPGK |
| | SEQ ID NO: | Chain 2 DNA | GAAGTGCAGCTGCTCGAGAGCGGGGG |
| | 146 | (huBG219P-E39) | CGGCCTCGTGCAACCTGGCGGGAGCC |
| | | | TGAGACTGAGCTGCGCCGCTAGCGGC |
| | | | TTCACCTTCAACGACTACATCATGAGC |
| | | | TGGGTGAGACAAGCCCCCGGCAAGGG |
| | | | CCTGGAGTGGGTGGCCACCGTGAGCA |
| | | | GCGGGGGCGACAACACCTACTACCCC |
| | | | GACAGCGTGAAGGGCAGATTCACCAT |
| | | | CAGCAGAGACAACAGCAAGAACACCC |
| | | | TGTACCTGCAGATGAACAGCCTGAGA |
| | | | GCCGAGGACACCGCCGTGTACTACTG |
| | | | CACAAGATACGGCGGCCTGGGCGCCA |
| | | | TGGACTACTGGGGCCAAGGCACCCTG |
| | | | GTGACCGTCAGCTCCGCTAGCACCAA |
| | | | GGGGCCCTCGGTCTTCCCCCTGGCACC |
| | | | CTCCTCCAAGAGTACTTCTGGGGGCAC |
| | | | AGCGGCCCTGGGCTGCCTGGTCAAGG |
| | | | ACTACTTCCCCGAACCGGTGACGGTGT |
| | | | CGTGGAACTCAGGCGCCCTGACCAGC |
| | | | GGCGTGCACACCTTCCCGGCTGTCCTA |
| | | | CAGTCCTCAGGACTCTACTCCCTCAGC |
| | | | AGCGTGGTGACCGTGCCCTCCAGCAG |
| | | | CTTGGGCACCCAGACCTACATCTGCA |
| | | | ACGTGAATCACAAGCCCAGCAACACC |
| | | | AAGGTGGACAAGAAAGTTGAGCCCAA |
| | | | ATCTTGTGACAAAACTCACACATGCCC |
| | | | ACCGTGCCCAGCACCTGAACTCCTGG |
| | | | GGGGACCGTCAGTCTTCCTCTTCCCCC |
| | | | CAAAACCCAAGGACACCCTCTACATC |
| | | | ACCCGGGAACCTGAGGTCACATGCGT |
| | | | GGTGGTGGACGTGAGCCACGAAGACC |
| | | | CTGAGGTCAAGTTCAACTGGTACGTG |
| | | | GACGGCGTGGAGGTGCATAATGCCAA |
| | | | GACAAAGCCGCGGGAGGAGCAGTACA |
| | | | ACAGCACGTACCGTGTGGTCAGCGTC |
| | | | CTCACCGTCCTGCACCAGGACTGGCTG |
| | | | AATGGCAAGGAGTACAAGTGCAAGGT |
| | | | CTCCAACAAAGCCCTCCCAGCCCCCAT |
| | | | CGAGAAAACCATCTCCAAAGCCAAAG |
| | | | GGCAGCCCCGAGAACCACAGGTGTAC |
| | | | ACCCTGCCCCCATCCCGGGAAGAGAT |
| | | | GACCAAGAACCAGGTCAGCCTGTCCT |
| | | | GCGCCGTCAAAGGCTTCTATCCCAGC |
| | | | GACATCGCCGTGGAGTGGGAGAGCAA |
| | | | TGGGCAGCCGGAGAACAACTACAAGA |
| | | | CCACGCCTCCCGTGCTGGACTCCGACG |
| | | | GCTCCTTCTTCCTCGTCAGCAAGCTCA |
| | | | CCGTGGACAAGAGCAGGTGGCAGCAG |
| | | | GGGAACGTCTTCTCATGCTCCGTGATG |
| | | | CATGAGGCTCTGCACAACCACTACAC |
| | | | CCAGAAGAGCCTCTCCCTGTCTCCGGG |
| | | | TAAATGA |
| | SEQ ID NO: | Chain 3 AA (light | DIQMTQSPSSLSASVGDRVTITCRASGNI |
| | 147 | chain) | HNYLTWYQQKPGKAPKLLIYNAKTLAS |
| | | | GVPSRFSGSGSGTDFTLTISSLQPEDFAT |
| | | | YYCQHFWSVPYTFGQGTKLEIKRTVAA |
| | | | PSVFIFPPSDEQLKSGTASVVCLLNNFYP |
| | | | REAKVQWKVDNALQSGNSQESVTEQD |
| | | | SKDSTYSLSSTLTLSKADYEKHKVYACE |
| | | | VTHQGLSSPVTKSFNRGEC |
| | SEQ ID NO: | Chain 3 DNA | GACATTCAGATGACACAGAGCCCTAG |
| | 148 | (light chain) | CAGCCTGAGCGCTAGCGTGGGCGACA |
| | | | GAGTGACCATCACCTGCAGAGCTAGC |
| | | | GGCAACATCCACAACTACCTGACCTG |
| | | | GTATCAGCAGAAGCCCGGCAAGGCCC |
| | | | CCAAGCTGCTGATCTACAACGCCAAG |
| | | | ACCCTGGCCAGCGGCGTGCCTAGCAG |
| | | | ATTCAGCGGCAGCGGCAGCGGCACCG |
| | | | ACTTCACCCTGACCATCAGCAGCCTGC |
| | | | AGCCCGAGGACTTCGCCACCTACTACT |
| | | | GTCAGCACTTCTGGAGCGTGCCCTACA |
| | | | CCTTCGGCCAAGGCACCAAGCTGGAG |
| | | | ATCAAGCGTACGGTGGCTGCACCATC |
| | | | TGTCTTCATCTTCCCGCCATCTGATGA |
| | | | GCAGTTGAAATCTGGAACTGCCTCTGT |
| | | | TGTGTGCCTGCTGAATAACTTCTATCC |
| | | | CAGAGAGGCCAAAGTACAGTGGAAGG |
| | | | TGGATAACGCCCTCCAATCGGGTAAC |
| | | | TCCCAGGAGAGTGTCACAGAGCAGGA |
| | | | CAGCAAGGACAGCACCTACAGCCTCA |
| | | | GCAGCACCCTGACGCTGAGCAAAGCA |
| | | | GACTACGAGAAACACAAAGTCTACGC |
| | | | CTGCGAAGTCACCCATCAGGGCCTGA |
| | | | GCTCGCCCGTCACAAAGAGCTTCAAC |
| | | | AGGGGAGAGTGTTGA |

TABLE 25
Summary table of multiple anti-human MUC1 reference Ab

| | | | | SEQ |
|---|---|---|---|---|
| | | | | ID |
| Seq Source | Information | Epitope | Sequence | NO |
| WHO | Gatipotuzumab- | tumor- | EVQLVESGGGLVQPGGSMRLSCVASGFPFS | 186 |
| Proposed | VH | specific | NYWMNWVRQAPGKGLEWVGEIRLKSNN | |
| INN: List | | carbohydrate/ | YTTHYAESVKGRFTISRDDSKNSLYLQMNS | |
| 118 WHO | | protein | LKTEDTAVYYCTRHYYFDYWGQGTLVTVS | |
| Drug | | mixed | S | |
| Information, | Gatipotuzumab- | epitope | DIVMTQSPLSNPVTPGEPASISCRSSKSLLHS | 187 |
| Vol. 31, No. | VL | | NGITYFFWYLQKPGQSPQLLIYQMSNLASG | |
| 4, 2017 | | | VPDRFSGSGSGTDFTLRISRVEAEDVGVYY | |
| | | | CAQNLELPPTFGQGTKVEIK | |
| WHO Drug | Clivatuzumab-VH | Conformational | QVQLQQSGAEVKKPGASVKVSCEASGYTF | 188 |
| Information, | | epitope | PSYVLHWVKQAPGQGLEWIGYINPYNDGT | |
| Vol. 29, No. | | dependent on | QYNEKFKGKATLTRDTSINTAYMELSRLRS | |
| 2, 2015 | | glycosylation | DDTAVYYCARGFGGSYGFAYWGQGTLVT | |
| Proposed | | | VSS | |
| INN: List | Clivatuzumab-VL | | DIQLTQSPSSLSASVGDRVTMTCSASSSVSS | 189 |
| 113 | | | SYLYWYQQKPGKAPKLWIYSTSNLASGVP | |
| | | | ARFSGSGSGTDFTLTISSLQPEDSASYFCHQ | |
| | | | WNRYPYTFGGGTRLEIK | |
| Ref: | HuVH- | PDTR | QVQLVQSGAEVKKPGASVKVSCKASGYTF | 190 |
| Immunology. | HMFG1_VH | peptide seq | SAYWIEWVRQAPGKGLEWVGEILPGSNNS | |
| 1993 Mar; | | | RYNEKFKGRVTVTRDTSTNTAYMELSSLRS | |
| 78(3): 364-70 | | | EDTAVYYCARSYDFAWFAYWGQGTLVTVS | |
| | | | S | |
| | HuVH- | | DIQMTQSPSSLSASVGDRVTITCKSSQSLLY | 191 |
| | HMFG1_VL | | SSNQKIYLAWYQQKPGKAPKLLIYWASTR | |
| | | | ESGVPSRFSGSGSGTDFTFTISSLQPEDIATY | |
| | | | YCQQYYRYPRTFGQGTKVEIK | |
| Ref: | 5F3-VH | Human | DVQVQESGPDLVKPSQSLSLTCTVTGHSIT | 192 |
| Cancer | | MUC1 SEA | RGSSWHWIRQFPGNKLEWMGYIHYGGGT | |
| Immunol | | | SYNPSLKSRISITRDTSKNQFFLQLNSVTTE | |
| Immunother. | | | DTATFFCARYSYDITYRWFFDVWGAGTTVI | |
| 2020 Jul; | | | VSS | |
| 69(7): 1337- | 5F3-VL | | DILLTQSPAILSVSPGERVSFSCRASQNIGTSI | 193 |
| 1352. | | | HWYQQRKNGSPRLLIKYASESISGIPSRFSG | |
| | | | SGSGTDFTLSINSVESEDMADYYCQQNNN | |
| | | | WPLTFGAGTKLELK | |
| Patent Pub.: | 3D1-VH | MUC1-C | EVQLVQSGAEVKKPGESLKISCKGSGYAFS | 194 |
| US | | (58 aa) | NFWMNWVRQMPGKGLEWMGQIYPGDGD | |
| 2018/0036441 | | | TNYNGKFKGQVTISADKSISTAYLQWSSLK | |
| A1 | | | ASDTAMYYCARSYYRSAWFAYWGQGTLV | |
| | | | TVSL | |
| | 3D1-VL | | EIVLTQSPDFQSVTPKEKVTITCRASQSIGTS | 195 |
| | | | IHWYQQKPDQSPKLLIKYASESISGVPSRFS | |
| | | | GSGSGTDFTLTINSLEAEDAATYYCQQSNN | |
| | | | WPLTFGQGTKVEIK | |

### Example 13. Generation and Production of MUC1 Targeting Antibodies

### Generation and Production of MUC1×CD16A Multi-Specific Antibody BG1222P

For construction of the MUC1×CD16A multi-specific antibody BG1222P, anti-CD16AVHH-BG526P and anti-MUC1 antibody huBG219P-E39 were assembled in a “2+1” IgG like bispecific format with facilitation of knob-into-hole (KiH) mutations for Fc dimerization (FIG. 15). Specifically, tandem BG526P VHHs with 2G4S linker (GGGGSGGGGS, SEQ ID NO: 72) in between were fused with N-terminal of the hinge region of human IgG1 constant regions carrying a T366W (EU numbering) mutation for a “knob” mutation, C220S (EU numbering) to remove the free cysteine, and M252Y/S254T/T256E for half-life elongation (chain 1, SEQ ID: 143-144). For the MUC1 binding arm, the humanized MUC1 antibody huBG219P-E39 VH region was fused to the constant regions of human IgG1 carrying T366S/L368A/Y407V (EU numbering) for “hole” mutations and M252Y/S254T/T256E for half-life elongation (chain 2, SEQ ID NO: 145-146). The light chain of BG1222P was made from the humanized MUC1 antibody huBG219P-E39 VL region fused to the constant region of human kappa chain (chain 3, SEQ ID NO: 147-148). Those constructs were prepared using an in-house developed expression vector or pcDNA3.4 with easy adapting sub-cloning sites.

All three plasmids were co-transfected into ExpiCHO-s cells (Thermofisher Scientific). The plasmid ratio was optimized to facilitate the downstream purification procedure by improving the purity of starting material. The bispecific antibody was captured with MabSelect SuRe Lx (Cytiva) first, and further polished by two ion-exchange columns. Capto S ImpAct and Capto Q ImpRes (Cytiva), to remove most impurities and aggregates. The final products were buffer exchanged to DPBS or histidine buffer using G25 desalting column (Cytiva), and stored at −80° C.

### Generation of Multiple Anti-Human MUC1 Reference Ab

Multiple anti-human MUC1 reference antibody sequences were extracted from published literature and patents, as summarized in Table 25. The above reference antibodies were constructed with an in house IgG1/Cκ eukaryotic expression vector.

For the afucosylated versions (referred with suffix -AF) of the antibodies, including reference antibodies above and huBG219P-E39-AF, if used in assays, they were generated with ExpiCHO transient expression system (Thermofisher Scientific). For inhibition of fucosylation, 2F-peracetyl-fucose (catalog #344827, EMD Millipore) was added to the growth medium at a final concentration of 100 μM before inoculation. The conditioned media was harvested and the afucosylated antibodies were purified using a MabSelect SuRe column (Cytiva) followed by a SPHP column (Cytiva). All the purified antibodies were buffer exchanged to DPBS via UF/DF and stored at −80° C. in small aliquots for assays later.

### Example 14. Binding Kinetics and Affinity Determination of MUC1×CD16A Multi-Specific Antibody

For affinity determination, a surface plasmon resonance (SPR) technology-based assay was developed to characterize the binding affinities of the bispecific antibody. Briefly, netrAvidin was immobilized onto a CM5 chip surface, then biotin-labeled anti human Fc VHH was flown over the surface and captured by immobilized netrAvidin. MUC1×CD16A multi-specific antibody was captured by anti-human Fc VHH/netrAvidin complex on the chip surface, and serial dilutions of CD16A or hMUC1-SEA-mFc (human MUC1-SEA domain linked with mouse IgG2a Fc) were flown over the surface and the binding response was calculated by subtracting RU from a reference flow-cell without injection of the bispecific antibody. The results of SPR-determined binding profiles of MUC1×CD16A multi-specific antibody are summarized in Table 26. The multi-specific antibody BG1222P showed high binding affinities to the targets human CD16A (158V and 158F) and hMUC1-SEA.

TABLE 26
SPR binding affinities of the multi-specific antibody to
human CD16A and hMUC1- SEA-mFc

| Analyte | kₒₙ (M⁻¹s⁻¹) | k_{off} (s⁻¹) | K_{D} (nM) |
|---|---|---|---|
| CD16A 158V | 8.86E+05 | 4.22E-05 | 4.77E-11 |
| CD16A 158F | 5.79E+05 | 8.52E-05 | 1.47E-10 |
| hMUC1-SEA-mFc | 1.17E+06 | 3.35E-05 | 2.86E-11 |

### Example 15. Determining the Binding Affinity of MUC1×CD16A Multi-Specific Antibody

### Binding Affinity of MUC1×CD16A Multi-Specific Antibody to MUC1 Expressed Cancer Cell Line

Purified BG1222P was determined the binding affinity to MUC1 expressed cancer cell line and NK92mi by FACS. Briefly, MUC1 expressed tumor cell line, T47D, was incubated with serially diluted purified BG1222P, washed, and incubated with anti-human IgG secondary Ab conjugated to APC. After incubation and washing, fluorescence was measured by flow cytometry. The BG1222P binding affinity to T47D is shown below in Table 27 and FIG. 16. The result indicates BG1222P has high binding affinity to MUC1 expressed cancer cell line.

TABLE 27
Cell binding affinity of MUC1xCD16A multi-specific
antibody BG1222P to T47D

| Antibody | Cell line | EC₅₀ (nM) |
|---|---|---|
| BG1222P | T47D | 28.07 |

### Binding Affinity of MUC1×CD16A Multi-Specific Antibody to NK92mi/CD16A F158 and NK92mi/CD16A V158 Cells

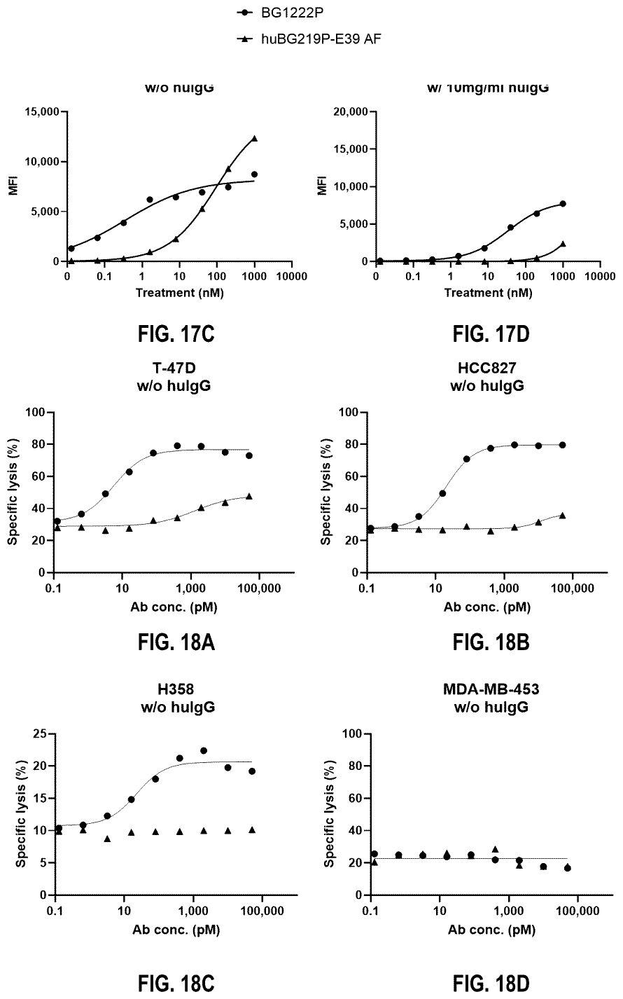

To evaluate the binding activity of BG1222P and huBG219P-E39-AF antibodies to CD16A expressed on living cells. NK92mi (ATCC) cells were engineered to over-express human CD16A (NK92mi/CD16A F158 and NK92mi/CD16A V158) by co-transducing expression plasmids containing CD16A (F158 or V158 allele) and FcRγ cDNAs. The purified and biotinylated bispecific antibodies were serially diluted and incubated with NK92mi/CD16A F158 or NK92mi/CD16A V158 cells for 45 mins at 37° C. in the presence or absence of 10 mg/ml human IgG. After washing twice with FACS buffer, diluted Alexa Fluor 647 streptavidin (Invitrogen #S32357) was added and incubated with the cells for 60 mins at 4° C. in the dark. After washing twice with FACS buffer, cells were resuspended with FACS buffer and acquired on BD FACS Celesta. Titration curves were generated using sigmoid dose-response of nonlinear fit from GraphPad, and the EC₅₀ of representative antibodies are shown in Table 28 and FIGS. 17A-17D. BG1222P exhibits a strong binding affinity to both F158 and V158 human CD16A overexpressing cell lines. Human IgG competition compromises the binding Emax of huBG219P-E39-AF, but not that of BG1222P. The results indicate the CD16A binding affinity of BG1222P can be better maintained than the binding affinity of huBG219P-E39-AF in the circulation or tumor where high levels of IgG exist. CD16A binding activity of BG1222P is less compromised by IgG competition than it is for huBG219P-E39-AF.

TABLE 28
Comparison of human CD16A over-expressing NK92mi binding affinity of
MUC1xCD16A multi-specific antibody and MUC1 antibody

| | | w/o (without) huIgG | | 10 mg/ml huIgG1 | |
|---|---|---|---|---|---|
| NK92-MI | Antibody | EC₅₀ (nM) | Emax (MFI) | EC₅₀ (nM) | Emax (MFI) |
| CD16A | BG1222P | 0.97 | 18895 | 7.99 | 18205 |
| F158 | huBG219P-E39-AF | N/A | 10318 | N/A | 3219 |
| CD16A | BG1222P | 0.35 | 8231 | 33.89 | 8101 |
| V158 | huBG219P-E39-AF | 94.14 | 12340 | N/A | 2387 |

N/A: EC₅₀ is not calculatable given the weak activity

### Example 16. Antibody-Dependent Cellular Cytotoxic Activity of MUC1×CD16A Multi-Specific Antibody

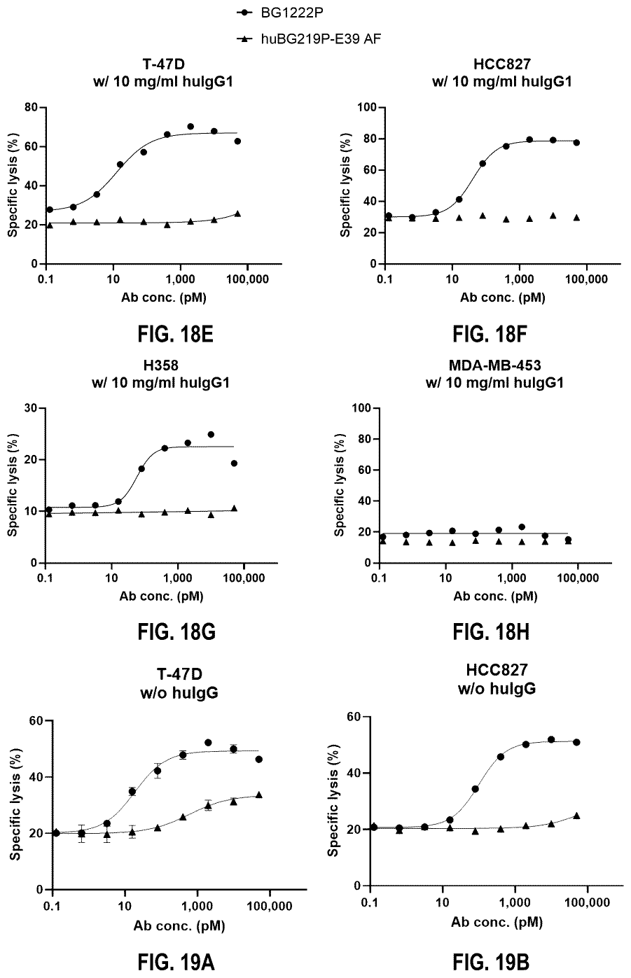

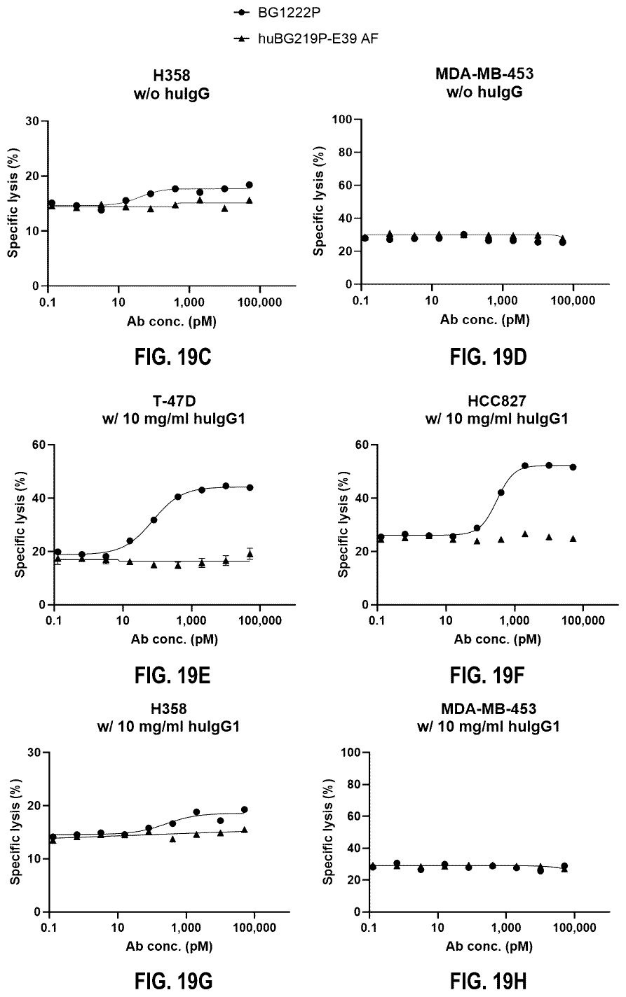

A nanoluc-release assay was set up to determine the antibody-dependent cellular cytotoxic activity of BG1222P and huBG219P-E39-AF antibodies on MUC1⁺ cells. The NK92mi/CD16A F158 and NK92mi/CD16A V158 cell lines were used as effector cells. Several cancer cell lines with different expression levels of MUC1-T-47D (MUC1 high), HCC827 (MUC1 medium), H358 (MUC1 low), and MDA-MB-453 (MUC1 negative)—were engineered to express nanoluc in the cell by retroviral transduction and were used as target cells. When the target cells were lysed by the effector cells, the nanoluc was released into the culture medium. Cytotoxicity was evaluated by measuring the nanoluc in the supernatant with the Nano-Glo Luciferase Assay kit (Promega, Madison, Wis.). Briefly, effector cells and target cells with a E:T ratio of 2:1 were added to V-bottom 96-well plates with a series of dilutions of bispecific antibodies in the presence or absence of 10 mg/ml human IgG1 and co-cultured for 20-24 hours at 37° C. The specific lysis was determined using the following equation: percentage of specific lysis=[luminescence (sample)−luminescence (spontaneous)]/[luminescence (maximum)−luminescence (spontaneous)]×100%. Luminescence (spontaneous) represents the luminescent counts from the supernatant of target cells without effector cells and antibodies. Luminescence (maximum) represents luminescent counts released after total cell lysis induced by the addition of Triton-X-100. As shown in Tables 29 and 30 and FIGS. 18A-19H, BG11222P induced the lysis of the MUC1 expressing cell lines in a dose-dependent manner but not the MUC1 negative cell line MDA-MB-453, in the presence or absence of human IgG. The results indicate the ADCC activity of BG1222P is superior to that of huBG219P-E39-AF.

TABLE 29
Comparison of antibody-dependent cellular cytotoxic activity of MUC1xCD16A
multi-specific antibody and MUC1 antibody mediated by NK92mi/CD16A F158 cells

| | | w/o huIgG | | 10 mg/ml huIgG1 | |
|---|---|---|---|---|---|
| Target cell | Antibody | EC₅₀ (pM) | Emax (%) | EC₅₀ (pM) | Emax (%) |
| T-47D | BG1222P | 5.6 | 76.8 | 12.6 | 66.9 |
| (MUC1 high) | huBG219P-E39-AF | 1302.0 | 48.2 | N/A | 25.7 |
| HCC827 | BG1222P | 20.4 | 79.7 | 41.0 | 78.7 |
| (MUC1 medium) | huBG219P-E39-AF | 11524.0 | 36.8 | N/A | 31.0 |
| H358 | BG1222P | 22.6 | 20.7 | 57.7 | 22.5 |
| (MUC1 low) | huBG219P-E39-AF | N/A | 10.2 | N/A | 10.7 |
| MDA-MB-453 | BG1222P | N/A | 26.1 | N/A | 22.8 |
| (MUC1 negative) | huBG219P-E39-AF | N/A | 25.7 | N/A | 16.0 |

N/A: EC₅₀ is not calculatable given the weak activity

TABLE 30
Comparison of antibody-dependent cellular cytotoxic activity of MUC1xCD16A
multi-specific antibody and MUC1 antibody mediated by NK92mi/CD16A V158 cells

| | | w/o huIgG | | 10 mg/ml huIgG1 | |
|---|---|---|---|---|---|
| Target cell | Antibody | EC₅₀ (pM) | Emax (%) | EC₅₀ (pM) | Emax (%) |
| T-47D | BG1222P | 18.9 | 49.3 | 73.3 | 44.3 |
| (MUC1 high) | huBG219P-E39-AF | 603.0 | 33.6 | N/A | 19.2 |
| HCC827 | BG1222P | 100.9 | 51.3 | 302.8 | 52.4 |
| (MUC1 medium) | huBG219P-E39-AF | N/A | 25.0 | N/A | 26.7 |
| H358 | BG1222P | 39.1 | 17.7 | 253.2 | 18.6 |
| (MUC1 low) | huBG219P-E39-AF | N/A | 15.6 | N/A | 15.5 |
| MDA-MB-453 | BG1222P | N/A | 30.2 | N/A | 30.8 |
| (MUC1 negative) | huBG219P-E39-AF | N/A | 29.8 | N/A | 29.2 |

N/A: EC₅₀ is not calculatable given the weak activity

### Example 17. Cell Lysis Activity of MUC1×CD16A Multi-Specific Antibody on MUC1 Expressing Cells in Human Whole Blood Assay

The nanoluc-release assay was also employed to evaluate cell lysis activity of BG1222P in the human whole blood. In brief, 100 μL/well of human whole blood from healthy donors were mixed with the target cells (2000 cells/well) described in Example 16 (T-47D/nanoluc, HCC827/nanoluc. H358/nanoluc, and MDA-MB-453/nanoluc) and a series of dilutions of bispecific antibodies in U-bottom 96-well plates. The total volume was 200 μL/well. After incubating at 37° C. for 18-20 hours, nanoluc released into the supernatant was measured using the Nano-Glo Luciferase Assay kit. The specific lysis was determined using the equation described in Example 16.

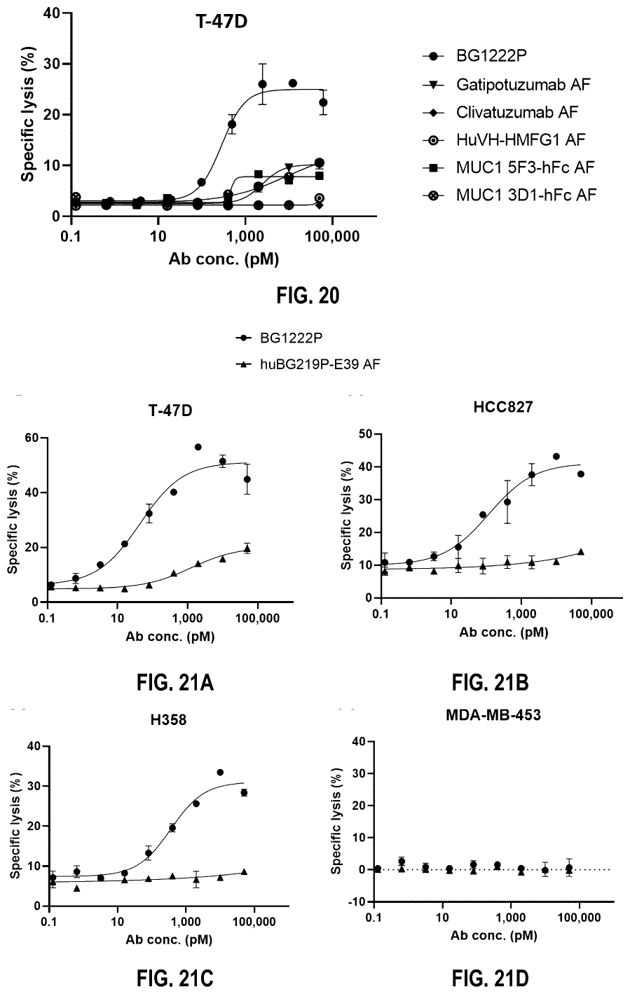

To compare the cell lysis activity of bispecific antibodies with that of the anti-MUC1 monoclonal IgG1 antibodies, the following antibodies with engineered Fc (afucosylation, -AF) were produced with reference to the published sequences: Gatipotuzumab-AF, Clivatuzunmab-AF, HuVH-HMFG1-AF, MUC1 5F3-hFc-AF, and MUC1 3D1-hFc-AF. The removal of core fucose (afucosylation) of the Fc glycan has been shown to highly increase FcγRIIIa binding affinity and consequently increase an antibody's cellular cytotoxic activity. These antibodies were compared with bispecific antibodies in the human whole blood assay described above using T-47D/nanoluc as the target cell. As shown in Table 31 and FIG. 20, BG1222P exhibits a much stronger cell lysis activity in EC₅₀ and Emax than the foregoing anti-MUC1 afucosylated antibodies.

Besides T47D cells, the cell lysis activity of BG1222P on target cells with different MUC1 expression levels was also evaluated in the human whole blood assay. As shown in Table 32 and FIGS. 21A-21D, BG1222P specifically induced the lysis of the MUC1 expressing cell lines but not the MUC1 negative cell line MDA-MB-453 in human whole blood in a dose-dependent manner. The results indicate that the cell lysis activity of BG1222P is superior to huBG219P-E39-AF.

TABLE 31
Comparison of cell lysis activity of MUC1xCD16A
multi-specific antibody and various MUC1
afucosylated antibodies in human whole blood

| | Cell lysis activity | |
|---|---|---|
| Antibody | EC₅₀ (pM) | Emax (%) |
| BG1222P | 285.20 | 24.99 |
| Gatipotuzumab-AF | 2463.00 | 10.18 |
| Clivatuzumab-AF | N/A | 2.31 |
| HuVH-HMFG1-AF | N/A | 3.59 |
| MUC1 5F3-hFc-AF | 464.30 | 7.78 |
| MUC1 3D1-hFc-AF | 8852.00 | 10.56 |

N/A: EC₅₀ is not calculatable given the weak activity

TABLE 32
Comparison of cell lysis activity of MUC1xCD16A multi-specific antibody and MUC1
antibody in human whole blood

| | | Cell lysis activity | |
|---|---|---|---|
| Target cell | Antibody | EC₅₀ (pM) | Emax (%) |
| T-47D | BG1222P | 43.9 | 51.2 |
| (MUC1 high) | huBG219P-E39-AF | 1254.0 | 20.2 |
| HCC827 | BG1222P | 117.7 | 41.2 |
| (MUC1 medium) | huBG219P-E39-AF | N/A | 14.1 |
| H358 | BG1222P | 357.6 | 31.1 |
| (MUC1 low) | huBG219P-E39-AF | N/A | 8.6 |
| MDA-MB-453 | BG1222P | N/A | 2.7 |
| (MUC1 negative) | huBG219P-E39-AF | N/A | 1.0 |

N/A: EC₅₀ is not calculatable given the weak activity

### Example 18. Phagocytosis Activity of MUC1×CD16A Multi-Specific Antibody

Human PBMC-derived M2 macrophage was used as the effector cell to assess the phagocytotic activity of BG1222P. The generation of M2 macrophages was performed according to the protocol described by Leidi et al. (Journal of immunology, (2009) 182(7), 4415-4422 Briefly, human PBMCs (Sailybio) were cultured in 6-well plates (Corning) in complete RPMI1640 media supplemented with 30 ng/ml human M-CSF (Peprotech) for 4 days. Adherent cells were retained by gently washing off non- and loose-adherent cells, with half of the media replaced, and cultured for 2-3 more days. For M2 polarization, 10 ng/ml IL-10 (Peprotech) was added during the last 48 h of culture.

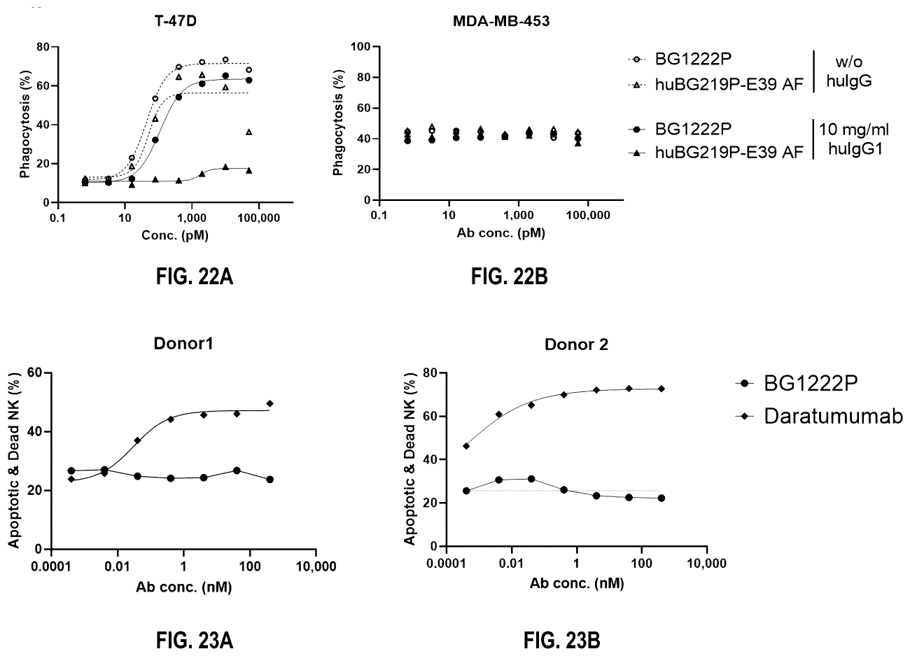

Target cells (T-47D and MDA-MB-453) were labeled with carboxyfluorescein succinimidyl ester (CFSE) (Life Technologies) according to the manufacturer's instructions. Target cells and M2 macrophages at a ratio of 2:1 were plated in U-bottom 96-well plates with bispecific antibodies in the presence or absence of 10 mg/ml human IgG. After 2 hours of incubation at 37° C., cells were stained with anti-CD11b-BV421 and subjected to flow cytometry. The percentage of macrophages that underwent antibody-dependent cellular phagocytosis of target cells was determined by FACS of double-positive cells (CFSE+ and CD11b+) following gating on CD11b+M2 macrophages. As shown in Table 33 and FIGS. 22A-22B, BG1222P showed better phagocytotic activity than huBG219P-E39-AF in a dose-dependent manner in MUC1 expressing cell line T47D either with or without the addition of human IgG. BG1222P did not exhibit activity on MUC1 negative cell line MDA-MB-453.

TABLE 33
Comparison of phagocytotic activity of MUC1xCD16A multi-specific antibody and
MUC1 antibody mediated by human macrophage M2

| | | w/o huIgG | | 10 mg/ml huIgG1 | |
|---|---|---|---|---|---|
| Target cell | Ab | EC₅₀ (pM) | Emax (%) | EC₅₀ (pM) | Emax (%) |
| T-47D | BG1222P | 44.3 | 71.5 | 111.8 | 63.4 |
| | huBG219P-E39-AF | 50.0 | 56.4 | N/A | 17.6 |
| MDA-MB-453 | BG1222P | N/A | 46.5 | N/A | 43.5 |
| | huBG219P-E39-AF | N/A | 47.9 | N/A | 42.9 |

N/A: EC₅₀ is not calculatable given the weak activity

### Example 19. NK Fratricide of MUC1×CD16A Multi-Specific Antibody

A flow cytometry-based assay was set up to determine the NK fratricide activity of BG1222P. Primary NK cells were isolated from PBMCs of healthy donors using an NK cell isolation kit from Miltenyi Biotec (Germany) according to the manufacturer's instructions. Isolated NK cells were cultured with a series of dilutions of BG1222P in V-bottom 96-well plates. Daratumumab, which is known to exhibit NK fratricide activity at the cellular level and in patients, was used as positive control. After 5 hours of incubation at 37° C., cells were stained with anti-CD3-BV421, annexin V-FITC, 7-AAD, and anti-CD56-AF647. The percentage of apoptotic and dead NK cells was determined by FACS of annexin V positive and double-positive cells (annexin V+ and 7-AAD+) following gating on CD3-CD56+NK cells. As shown in FIGS. 23A-23B, BG1222P showed no fratricide tendency in NK cells from either of two donors. In contrast, daratumumab exhibited dose-dependent killing activity on NK cells from both donors.

### Example 20. Pharmacokinetics Profile of MUC1×CD16A Multi-Specific Antibody in Cynomolgus

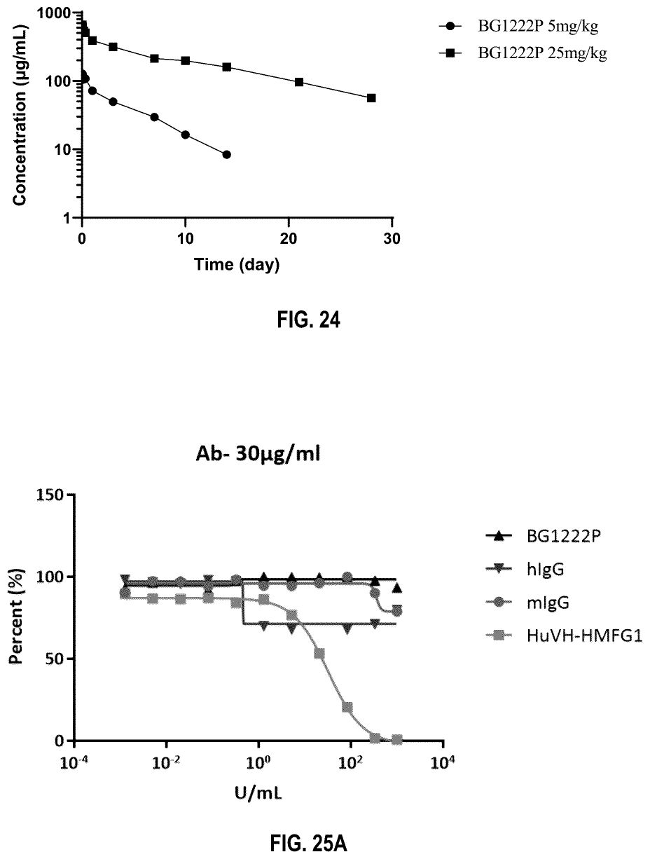

Blood samples were collected from Cynomolgus at 0, 0.5 hours, 1 hours, 4 hours, 8 hours, 1, 3, 7, 10, 14, 21, and 28 days after 5 mg/kg or 25 mg/kg intravenous infusion of BG1222P, followed by centrifugation (4° C., 3000×g, 15 min) to separate serum. The concentrations of BG1222P were measured by an in-house developed ELISA ligand binding method. Briefly, HuMUC1-SEA-mFc was used as a capture reagent, and biotin labelled CD16A-V158 with His tag was used as the detection reagent for BG1222P. The obtained pharmacokinetics profiles and parameters are shown in FIG. 24 and Table 34, respectively. In the 5 mg/kg dosing group, BG1222P was below the lower limit of quantification (0.78 μg/ml) on day 21 post-dose. Anti-drug antibody (ADA) was detected in serum since day 10 in the 5 mg/kg dosing group, which indicated a potential influence on the pharmacokinetic curve. In the 25 mg/kg dosing group, BG1222P exhibited a much lower clearance with minimal ADA influence. Even under these circumstances, a relatively long terminal elimination phase half-life of BG1222P ranging from 4.25 to 10.2 days after a 5-25 mg/kg dose was observed. The clearance ranging from 4.3-9.1 days suggests BG1222P was slowly cleared from body. The distribution volume (V_{z}) was close to the physiologic serum volume in Cynomolgus indicating BG1222P was primarily located in the serum volume.

TABLE 34
Pharmacokinetics parameters of MUC1xCD16A multi-specific
antibody in Cynomolgus after i.v. infusion
PK Parameter after i.v. infusion of BG1222P in Cyno (n = 1/group)

| PK Parameter | 5 mg/kg | 25 mg/kg |
|---|---|---|
| T_{1/2} (day) | 4.25 | 10.2 |
| Cmax (μg/mL) | 127 | 661 |
| AUClast (day * μg/mL) | 495 | 5001 |
| AUCINF (day * μg/mL) | 546.3 | 5830 |
| AUC_% Extrap (%) | 9.5 | 14.2 |
| CL (mL/day/kg) | 9.1 | 4.3 |
| V_{z} (mL/kg) | 56.1 | 62.4 |

Note:
non-compartment model was used to calculate the pharmacokinetics parameters

TABLE 35
Sequences of amino acid linker for scFv

| SEQ ID NO | note | Sequence |
|---|---|---|
| SEQ ID NO: 70 | AA linker | GGGGSGGGGSGGGGSGGGGS |
| SEQ ID NO: 71 | AA linker | GGGGSGGGGSGGGGS |
| SEQ ID NO: 72 | AA linker | GGGGSGGGGS |

### Example 21. MUC1×CD16A Multi-Specific Antibody Shows Reduced Interference by Soluble MUC1

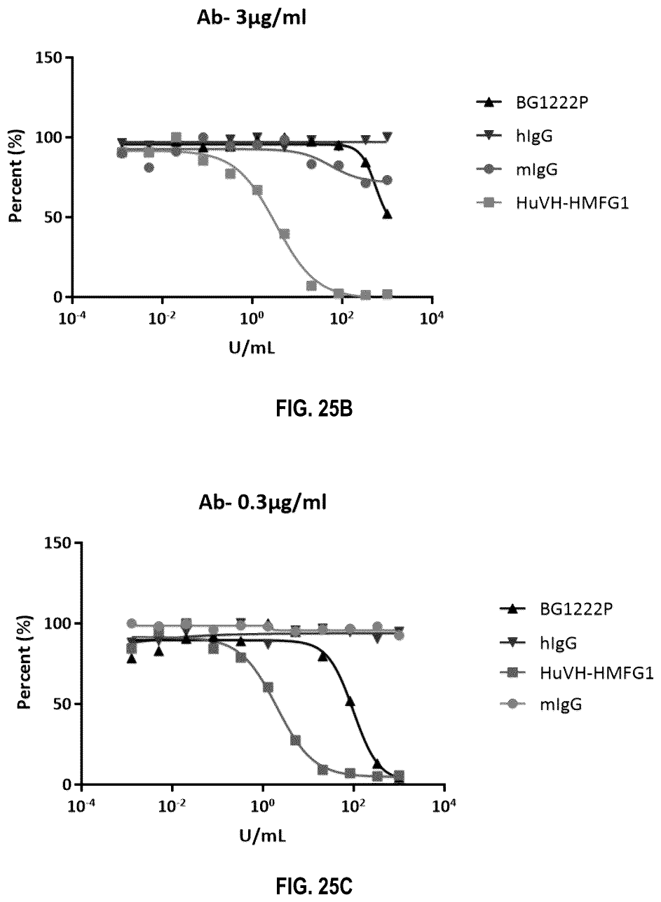

The influence of the presence of soluble MUC1 on the specific binding of MUC1×CD16A multi-specific antibody to MUC1 expressing cells was determined by competitive FACS assay. Briefly, human MUC1 expressing cells were incubated with 30, 3, or 0.3 μg/ml BG1222P in the presence of serially diluted soluble MUC1 (Shanghai Linc-Bio Science Co. LTD). After washing and incubation with anti-human IgG secondary antibody, fluorescence was measured by flow cytometry. IC₅₀ values for soluble MUC1 blocking BG1222P binding with MUC1 expressing cells are shown in Table 36. and the blocking curves are shown in FIGS. 25A-25C. The profiles indicate that the binding of HuVH-HMFG to MUC1-expressing cells can be easily interfered with at high, medium, and low antibody concentrations (i.e., 30, 3, 0.3 g/ml), but BG1222P binding is only slightly interfered with at the low antibody concentration (0.3 μg/ml) (FIGS. 25A-25C). Collectively, the profiles in FIG. 25 indicate that the binding of BG1222P to MUC1-expressing cells showed significantly reduced interference by soluble MUC1 compared to HuVH-HMFG1, which targets the MUC1 membrane distal portion.

TABLE 36
IC₅₀ of soluble MUC1 blocking activity
IC₅₀ (U/mL)

| Antibody | 30 μg/ml | 3 μg/ml | 0.3 μg/ml |
|---|---|---|---|
| BG1222P | N.A. | ~559.9 | 98.52 |
| HMFG1 | 31.91 | 3.38 | 2.03 |
| hIgG | N.A. | N.A. | N.A. |
| mIgG | N.A. | N.A. | N.A. |

(N.A.: Not available. IC₅₀ data cannot be fitted well and retrieved due to the extremely low interference, as shown from the binding curves in FIGS. 25A-25C.)

1. A multi-specific antibody or antigen-binding fragment thereof, comprising a first antigen binding domain that specifically binds to human MUC1 and a second antigen binding domain that specifically binds to human CD16A.

2. The multi-specific antibody or antigen-binding fragment of claim 1, wherein the second antigen binding domain has high selectivity over human CD16B.

3. The multi-specific antibody or antigen-binding fragment of claim 1, wherein the first antigen binding domain that specifically binds to human MUC1 comprises:
(i). a heavy chain variable region (VH) that comprises (a) a heavy chain complementarity determining region 1 (HCDR1) of SEQ ID NO: 24, (b) a HCDR2 of SEQ ID NO: 25, and (c) a HCDR3 of SEQ ID NO: 26, and a light chain variable region (VL) that comprises: (d) a light chain complementarity determining region 1 (LCDR1) of SEQ ID NO: 27, (e) a LCDR2 of SEQ ID NO: 28, and (f) a LCDR3 of SEQ ID NO: 29;
(ii). a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 65, and (f) a LCDR3 of SEQ ID NO: 29;
(iii). a heavy chain variable region (VH) that comprises (a) a HCDR1 of SEQ ID NO: 4, (b) a HCDR2 of SEQ ID NO: 5, and (c) a HCDR3 of SEQ ID NO: 6, and a light chain variable region (VL) that comprises: (d) a LCDR1 of SEQ ID NO: 7, (e) a LCDR2 of SEQ ID NO: 8, and (f) a LCDR3 of SEQ ID NO: 9; or
(iv). a heavy chain variable region (VH) that comprises (a) a HCDR1 of SEQ ID NO: 14, (b) a HCDR2 of SEQ ID NO: 15, and (c) a HCDR3 of SEQ ID NO: 16, and a light chain variable region (VL) that comprises: (d) a LCDR1 of SEQ ID NO: 17, (e) a LCDR2 of SEQ ID NO: 18, and (f) a LCDR3 of SEQ ID NO: 19.

4. The multi-specific antibody or antigen-binding fragment of claim 1, wherein the first antigen binding domain comprises:
(i). a heavy chain variable region (VH) comprising an amino acid sequence at least 90, 91, 92, 93, 94, 95, 96, 97, 98, or 99% identical to SEQ ID NO: 30, and a light chain variable region (VL) comprising an amino acid sequence at least 90, 91, 92, 93, 94, 95, 96, 97, 98, or 99% identical to SEQ ID NO: 31;
(ii). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 62;
(iii). a heavy chain variable region (VH) comprising an amino acid sequence at least 90, 91, 92, 93, 94, 95, 96, 97, 98 or 99% identical to SEQ ID NO: 61, and a light chain variable region (VL) comprising an amino acid sequence at least 90, 91, 92, 93, 94, 95, 96, 97, 98, or 99% identical to SEQ ID NO: 66;
(iv). a heavy chain variable region (VH) comprising an amino acid sequence at least 90, 91, 92, 93, 94, 95, 96, 97, 98, or 99% identical to SEQ ID NO: 61, and a light chain variable region (VL) comprising an amino acid sequence at least 90, 91, 92, 93, 94, 95, 96, 97, 98, or 99% identical to SEQ ID NO: 68;
(v). a heavy chain variable region (VH) comprising an amino acid sequence at least 90, 91, 92, 93, 94, 95, 96, 97, 98 or 99% identical to SEQ ID NO: 10, and a light chain variable region (VL) comprising an amino acid sequence at least 90, 91, 92, 93, 94, 95, 96, 97, 98, or 99% identical to SEQ ID NO: 11; or
(vi). a heavy chain variable region (VH) comprising an amino acid sequence at least 90, 91, 92, 93, 94, 95, 96, 97, 98 or 99% identical to SEQ ID NO: 20, and a light chain variable region (VL) comprising an amino acid sequence at least 90, 91, 92, 93, 94, 95, 96, 97, 98, or 99% identical to SEQ ID NO: 21.

5. The multi-specific antibody or antigen-binding fragment of claim 1, wherein one, two, three, four, five, six, seven, eight, nine, or ten amino acids within one or more of SEQ ID NO: 30, 31, 61, 62, 66, 68, 10, 11, 20, and 21 have been inserted, deleted, or substituted.

6. The multi-specific antibody or antigen-binding fragment of claim 1, wherein the first antigen binding domain comprises:
(i). a heavy chain variable region (VH) that comprises SEQ ID NO: 30, and a light chain variable region (VL) that comprises SEQ ID NO: 31;
(ii). a heavy chain variable region (VH) that comprises SEQ ID NO: 61, and a light chain variable region (VL) that comprises SEQ ID NO: 62;
(iii). a heavy chain variable region (VH) that comprises SEQ ID NO: 61, and a light chain variable region (VL) that comprises SEQ ID NO: 66;
(iv). a heavy chain variable region (VH) that comprises SEQ ID NO: 61, and a light chain variable region (VL) that comprises SEQ ID NO: 68;
(v). a heavy chain variable region (VH) that comprises SEQ ID NO: 10, and a light chain variable region (VL) that comprises SEQ ID NO: 11; or
(vi). a heavy chain variable region (VH) that comprises SEQ ID NO: 20, and a light chain variable region (VL) that comprises SEQ ID NO: 21.

7. The multi-specific antibody or antigen-binding fragment of claim 1, wherein the second antigen binding domain that specifically binds to human CD16A comprises:
(i). a heavy chain variable region (VH) that comprises (a) a HCDR1 of SEQ ID NO: 109, (b) a HCDR2 of SEQ ID NO: 110, and (c) a HCDR3 of SEQ ID NO: 111; or
(ii). a heavy chain variable region (VH) that comprises (a) a HCDR1 of SEQ ID NO: 109, (b) a HCDR2 of SEQ ID NO: 114, and (c) a HCDR3 of SEQ ID NO: 111.

8. The multi-specific antibody or antigen-binding fragment of claim 1, wherein the second antigen binding domain comprises:
(i). a heavy chain variable region (VH) comprising an amino acid sequence at least 90, 91, 92, 93, 94, 95, 96, 97, 98, or 99% identical to SEQ ID NO: 112;
(ii). a heavy chain variable region (VH) comprising an amino acid sequence at least 90, 91, 92, 93, 94, 95, 96, 97, 98, or 99% identical to SEQ ID NO: 115;
(iii). a heavy chain variable region (VH) comprising an amino acid sequence at least 90, 91, 92, 93, 94, 95, 96, 97, 98, or 99% identical to SEQ ID NO: 117; or
(iv). a heavy chain variable region (VH) comprising an amino acid sequence at least 90, 91, 92, 93, 94, 95, 96, 97, 98, or 99% identical to SEQ ID NO: 119.

9. The multi-specific antibody or antigen-binding fragment of claim 1, wherein one, two, three, four, five, six, seven, eight, nine, or ten amino acids within one or more of SEQ ID NO: 112, 115, 117, and 119 have been inserted, deleted, or substituted.

10. The multi-specific antibody or antigen-binding fragment of claim 1, wherein the second antigen binding domain comprises:
(i). a heavy chain variable region (VH) that comprises SEQ ID NO: 112;
(ii). a heavy chain variable region (VH) that comprises SEQ ID NO: 115;
(iii). a heavy chain variable region (VH) that comprises SEQ ID NO: 117; or
(iv). a heavy chain variable region (VH) that comprises SEQ ID NO: 119.

11. The multi-specific antibody or antigen-binding fragment of claim 1, wherein;
(i). the first antigen binding domain that specifically binds to human MUC1 comprises: a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 28, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(ii). the first antigen binding domain that specifically binds to human MUC1 comprises: a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 65, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(iii). the first antigen binding domain that specifically binds to human MUC1 comprises: a heavy chain variable region (VH) hat comprises (a) a HCDR1 of SEQ ID NO: 4, (b) a HCDR2 of SEQ ID NO: 5, and (c) a HCDR3 of SEQ ID NO: 6, and a light chain variable region (VL) that comprises: (d) a LCDR1 of SEQ ID NO: 7, (e) a LCDR2 of SEQ ID NO: 8, and (f) a LCDR3 of SEQ ID NO: 9; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(iv). a heavy chain variable region (VH) that comprises (a) a HCDR1 of SEQ ID NO: 14, (b) a HCDR2 of SEQ ID NO: 15, and (c) a HCDR3 of SEQ ID NO: 16, and a light chain variable region (VL) that comprises: (d) a LCDR1 of SEQ ID NO: 17, (e) a LCDR2 of SEQ ID NO: 18, and (f) a LCDR3 of SEQ ID NO: 19; and the second antigen binding domain that specifically binds to human CD16A comprises: (a) a HCDR1 of SEQ ID NO: 109, (b) a HCDR2 of SEQ ID NO: 110, and (c) a HCDR3 of SEQ ID NO: 111;
(v). the first antigen binding domain that specifically binds to human MUC1 comprises: a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 28, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111;
(vi). the first antigen binding domain that specifically binds to human MUC1 comprises: a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 65, and (f) a LCDR3 of SEQ ID NO: 29; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111;
(vii). the first antigen binding domain that specifically binds to human MUC1 comprises: a heavy chain variable region (VH) that comprises (a) a HCDR1 of SEQ ID NO: 4, (b) a HCDR2 of SEQ ID NO: 5, and (c) a HCDR3 of SEQ ID NO: 6, and a light chain variable region (VL) that comprises: (d) a LCDR1 of SEQ ID NO: 7, (e) a LCDR2 of SEQ ID NO: 8, and (f) a LCDR3 of SEQ ID NO: 9; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111; or
(viii). a heavy chain variable region (VH) that comprises (a) a HCDR1 of SEQ ID NO: 14, (b) a HCDR2 of SEQ ID NO: 15, and (c) a HCDR3 of SEQ ID NO: 16, and a light chain variable region (VL) that comprises: (d) a LCDR1 of SEQ ID NO: 17, (e) a LCDR2 of SEQ ID NO: 18, and (f) a LCDR3 of SEQ ID NO: 19; and the second antigen binding domain that specifically binds to human CD16A comprises: (a) a HCDR1 of SEQ ID NO: 109, (b) a HCDR2 of SEQ ID NO: 114, and (c) a HCDR3 of SEQ ID NO: 111.

12. The multi-specific antibody or antigen-binding fragment of claim 1, wherein;
(i). the first antigen binding domain that specifically binds to human MUC1 comprises:
a) a heavy chain variable region (VH) that comprises SEQ ID NO: 30, and a light chain variable region (VL) that comprises SEQ ID NO: 31;
b) a heavy chain variable region (VH) that comprises SEQ ID NO: 61, and a light chain variable region (VL) that comprises SEQ ID NO: 62;
c) a heavy chain variable region (VH) that comprises SEQ ID NO: 61, and a light chain variable region (VL) that comprises SEQ ID NO: 66;
d) a heavy chain variable region (VH) that comprises SEQ ID NO: 61, and a light chain variable region (VL) that comprises SEQ ID NO: 68;
e) a heavy chain variable region (VH) that comprises SEQ ID NO: 10, and a light chain variable region (VL) that comprises SEQ ID NO: 11; or
f) a heavy chain variable region (VH) that comprises SEQ ID NO: 20, and a light chain variable region (VL) that comprises SEQ ID NO: 21;
(ii). and the second antigen binding domain that specifically binds to human CD16A comprises:
a) a heavy chain variable region (VH) that comprises (a) a HCDR1 of SEQ ID NO: 109, (b) a HCDR2 of SEQ ID NO: 110, (c) a HCDR3 of SEQ ID NO: 111; or
b) a heavy chain variable region (VH) that comprises (a) a HCDR1 of SEQ ID NO: 109, (b) a HCDR2 of SEQ ID NO: 114, (c) a HCDR3 of SEQ ID NO: 111.

13. The multi-specific antibody or antigen-binding fragment of claim 1, wherein;
(i). the first antigen binding domain that specifically binds to human MUC1 comprises:
a) a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) that comprises: (d) a LCDR1 of SEQ ID NO: 27, (e) a LCDR2 of SEQ ID NO: 28, and (f) a LCDR3 of SEQ ID NO: 29;
b) a heavy chain variable region (VH) that comprises (a) a HCDR1 of SEQ ID NO: 24, (b) a HCDR2 of SEQ ID NO: 25, and (c) a HCDR3 of SEQ ID NO: 26, and a light chain variable region (VL) hat comprises: (d) a LCDR1 of SEQ ID NO: 27, (e) a LCDR2 of SEQ ID NO: 65, and (f) a LCDR3 of SEQ ID NO: 29;
c) a heavy chain variable region (VH) that comprises (a) a HCDR1 of SEQ ID NO: 4, (b) a HCDR2 of SEQ ID NO: 5, and (c) a HCDR3 of SEQ ID NO: 6, and a light chain variable region (VL) that comprises: (d) a LCDR1 of SEQ ID NO: 7, (e) a LCDR2 of SEQ ID NO: 8, and (f) a LCDR3 of SEQ ID NO: 9; or
d) a heavy chain variable region (VH) that comprises (a) a HCDR1 of SEQ ID NO: 14, (b) a HCDR2 of SEQ ID NO: 15, and (c) a HCDR3 of SEQ ID NO: 16, and a light chain variable region (VL) that comprises: (d) a LCDR1 of SEQ ID NO: 17, (e) a LCDR2 of SEQ ID NO: 18, and (f) a LCDR3 of SEQ ID NO: 19;
(ii). and the second antigen binding domain that specifically binds to human CD16A comprises:
a) a heavy chain variable region (VH) that comprises SEQ ID NO: 112;
b) a heavy chain variable region (VH) that comprises SEQ ID NO: 115;
c) a heavy chain variable region (VH) that comprises SEQ ID NO: 117; or
d) a heavy chain variable region (VH) that comprises SEQ ID NO: 119.

14. The multi-specific antibody or antigen-binding fragment of claim 1, wherein;
(i). the first antigen binding domain that specifically binds to human MUC1 comprises:
a) a heavy chain variable region (VH) that comprises SEQ ID NO: 30, and a light chain variable region (VL) that comprises SEQ ID NO: 31;
b) a heavy chain variable region (VH) that comprises SEQ ID NO: 61, and a light chain variable region (VL) that comprises SEQ ID NO: 62;
c) a heavy chain variable region (VH) that comprises SEQ ID NO: 61, and a light chain variable region (VL) that comprises SEQ ID NO: 66;
d) a heavy chain variable region (VH) that comprises SEQ ID NO: 61, and a light chain variable region (VL) that comprises SEQ ID NO: 68;
e) a heavy chain variable region (VH) that comprises SEQ ID NO: 10, and a light chain variable region (VL) that comprises SEQ ID NO: 11; or
f) a heavy chain variable region (VH) that comprises SEQ ID NO: 20, and a light chain variable region (VL) that comprises SEQ ID NO: 21;
(ii). and the second antigen binding domain that specifically binds to human CD16A comprises:
a) a heavy chain variable region (VH) that comprises SEQ ID NO: 112;
b) a heavy chain variable region (VH) that comprises SEQ ID NO: 115;
c) a heavy chain variable region (VH) that comprises SEQ ID NO: 117; or
d) a heavy chain variable region (VH) that comprises SEQ ID NO: 119.

15. The multi-specific antibody or antigen-binding fragment of claim 1, which is a monoclonal antibody, a chimeric antibody, a humanized antibody, a human engineered antibody, a single chain antibody (scFv), a Fab fragment, a Fab′ fragment, or a F(ab′)2 fragment.

16. The multi-specific antibody or antigen-binding fragment of claim 1, wherein the multi-specific antibody is a bispecific antibody.

17. The multi-specific antibody or antigen-binding fragment of claim 1, wherein the multi-specific antibody is BG1222P comprising SEQ ID NO: 143, SEQ ID NO: 145, and SEQ ID NO: 147.

18. The multi-specific antibody or antigen-binding fragment of claim 1, wherein the antibody or antigen-binding fragment thereof has antibody dependent cellular cytotoxicity (ADCC) or complement dependent cytotoxicity (CDC).

19. The multi-specific antibody or antigen-binding fragment of claim 1, wherein the antibody or antigen-binding fragment thereof has reduced glycosylation or no glycosylation or is hypofucosylated.

20. The multi-specific antibody or antigen-binding fragment of claim 1, wherein the antibody or antigen-binding fragment thereof comprises increased bisecting GlcNac structures.

21. The multi-specific antibody or antigen-binding fragment of claim 1, wherein the Fc domain is an IgG1 with reduced effector function.

22. The multi-specific antibody or antigen-binding fragment of claim 1, wherein the Fc domain is an IgG4.

23. A pharmaceutical composition comprising the multi-specific antibody or antigen-binding fragment thereof of claim 1 and a pharmaceutically acceptable carrier.

24. The pharmaceutical composition of claim 23, comprising histidine/histidine HCl, trehalose dihydrate, and polysorbate 20.

25. An isolated nucleic acid that encodes the multi-specific antibody or antigen-binding fragment of claim 1.

26. A vector comprising the nucleic acid of claim 25.

27. A host cell comprising the nucleic acid of claim 25.

28. A process for producing a multi-specific antibody or antigen-binding fragment thereof, comprising cultivating the host cell of claim 27 and recovering the antibody or antigen-binding fragment from the culture.

29. A host cell comprising the vector of claim 26.

30. A method of treating cancer comprising administering to a patient in need an effective amount of the multi-specific antibody or antigen-binding fragment of claim 1.

31. The method of claim 25, wherein the cancer is gastric cancer, colon cancer, pancreatic cancer, breast cancer, head and neck cancer, kidney cancer, liver cancer, lung cancer, small cell lung cancer, non-small cell lung cancer, ovarian cancer, skin cancer, mesothelioma, lymphoma, leukemia, myeloma and sarcoma.


SEQUENCE LISTING (195 sequences)

SEQ ID NO: 1 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANL

SEQ ID NO: 2 - protein, 120 aa, Homo sapiens
LSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAG

SEQ ID NO: 3 - protein, 449 aa, Macaca fascicularis
MTPGTQSPFFLLLILTVLTAATVPEPTTVVTGSGHTNSTPGGEKETSATQRSSMPISTKNAVSMTSRLSSHSPVSGSSTTQGQDVTLALAMESATGSATTLGHVVTSAPDTSAAPGSTGPPAHVVTSAPDTSAAPGSTGPPAHVVTSAPDTVTSASDSASGSASTLVHSTTSARATTTPASKSTPFSIPSHHSDTPTTLASHSTKTDASSTHHSTVPPFTSSNHSTSPQLSLGVSFFFLSFHISNLQFNSSLEDPSTNYYQQLQRDISELFLQIYKQGDFLGLSNIMFRPGSVVVQSTLVFREGTTNVHDVETQFNQRKTEAASRYNLTISDISVRDVPFPFSAQTGAGVPGWGIALLVLVCVLVVMAIVYFIALAVCQCRQKNYRQLDIFPARDAYHPMSEYPTYHTHGRYAPAGGTNRSPYEEVSAGNGGSSLSYTNPAVAATSANL

SEQ ID NO: 4 - protein, 5 aa, synthetic construct
  REGION 1..5: note = Synthetic
TYTMH

SEQ ID NO: 5 - protein, 17 aa, synthetic construct
  REGION 1..17: note = Synthetic
YINPGSDYTHYNQKFKD

SEQ ID NO: 6 - protein, 11 aa, synthetic construct
  REGION 1..11: note = Synthetic
QGNYAAYYFDY

SEQ ID NO: 7 - protein, 11 aa, synthetic construct
  REGION 1..11: note = Synthetic
RASGNIHNFLV

SEQ ID NO: 8 - protein, 7 aa, synthetic construct
  REGION 1..7: note = Synthetic
NAKTLTD

SEQ ID NO: 9 - protein, 9 aa, synthetic construct
  REGION 1..9: note = Synthetic
QNFWNVPFT

SEQ ID NO: 10 - protein, 120 aa, synthetic construct
  REGION 1..120: note = Synthetic
QVQLLQSGAELARPGASVKMSCKASGYTFTTYTMHWIKQRPGQGLEWIGYINPGSDYTHYNQKFKDKATLTADKSSSTAYMHLYSLTSEDSAVYYCARQGNYAAYYFDYWGQGTTLTVSS

SEQ ID NO: 11 - protein, 107 aa, synthetic construct
  REGION 1..107: note = Synthetic
DIQMTQSPASLSASVGETVTITCRASGNIHNFLVWYQQKQGKSPQLLVYNAKTLTDGVPSRFSGSGSGTQYSLKIKSLQPEDFGTYYCQNFWNVPFTFGSGTKLEIK

SEQ ID NO: 12 - DNA, 360 bp, synthetic construct
  misc_feature 1..360: note = Synthetic
  source 1..360: mol_type = other DNA
caggtccagttgttacagtctggggctgaactggcaagacctggggcctcagtgaagatgtcctgcaaggcttctggttacacctttactacctacacgatgcactggataaaacagaggcctgggcagggtctggaatggattggatacattaatcctggcagtgattatactcattacaatcagaaattcaaagacaaggccacattgactgcagacaaatcctccagcacagcctacatgcacctgtacagcctgacatctgaggactctgcagtctattactgtgcaagacagggcaactatgccgcgtactactttgactactggggccaaggcaccactctcacagtctcctca

SEQ ID NO: 13 - DNA, 321 bp, synthetic construct
  misc_feature 1..321: note = Synthetic
  source 1..321: mol_type = other DNA
gacatccagatgactcagtctccagcctccctatctgcatctgtgggagaaactgtcaccatcacatgtcgagcaagtgggaatattcacaattttttagtatggtatcagcagaaacagggaaaatctcctcagctcctggtctataatgcaaaaaccttaacagatggtgtgccatcaaggttcagtggcagtggatcaggaacacaatattctctcaagatcaagagcctgcagcctgaagattttgggacttattactgtcaaaatttttggaatgttccattcacgttcggctcggggacaaagttggaaataaaa

SEQ ID NO: 14 - protein, 5 aa, synthetic construct
  REGION 1..5: note = Synthetic
NYWMN

SEQ ID NO: 15 - protein, 17 aa, synthetic construct
  REGION 1..17: note = Synthetic
EIDPSDSYTNNNQKFKD

SEQ ID NO: 16 - protein, 8 aa, synthetic construct
  REGION 1..8: note = Synthetic
GRRTGFAY

SEQ ID NO: 17 - protein, 11 aa, synthetic construct
  REGION 1..11: note = Synthetic
RASENIYSNLA

SEQ ID NO: 18 - protein, 7 aa, synthetic construct
  REGION 1..7: note = Synthetic
AATNLAD

SEQ ID NO: 19 - protein, 9 aa, synthetic construct
  REGION 1..9: note = Synthetic
QHFWGTPYT

SEQ ID NO: 20 - protein, 117 aa, synthetic construct
  REGION 1..117: note = Synthetic
EVQLQQSGAELVKPGASVKISCKASGYTFTNYWMNWVRQRPGQGLEWIGEIDPSDSYTNNNQKFKDKATLTVDKSSSTAYMQLSSLTSEDSAVYYCARGRRTGFAYWGQGTLVTVSS

SEQ ID NO: 21 - protein, 107 aa, synthetic construct
  REGION 1..107: note = Synthetic
DIQMTQSPASLSVSVGETVTITCRASENIYSNLAWYQKKQGKSPQLLVYAATNLADGVPSRFSGSGSGTQYSLKINSLQSEDFGNYYCQHFWGTPYTFGGGTKLEIK

SEQ ID NO: 22 - DNA, 351 bp, synthetic construct
  misc_feature 1..351: note = Synthetic
  source 1..351: mol_type = other DNA
gaggtccagcttcagcagtctggggctgagctggtgaagcctggggcttcagtgaagatatcctgcaaggcgtctggctacacgttcaccaactactggatgaactgggtgaggcagaggcctggacaaggccttgagtggatcggagagatcgatccttctgatagttatactaacaacaatcaaaagttcaaggacaaggccacattgactgtagacaaatcctccagcacagcctacatgcagctcagcagcctgacatctgaggactctgcggtctattactgtgcaagagggaggaggacggggtttgcttactggggccaagggactctggtcactgtctcgagc

SEQ ID NO: 23 - DNA, 321 bp, synthetic construct
  misc_feature 1..321: note = Synthetic
  source 1..321: mol_type = other DNA
gacatccagatgacccagtctccagcctccctatctgtatctgtgggagaaactgtcaccatcacatgtcgagcaagtgagaatatttacagtaatttagcatggtatcagaagaaacagggaaaatctcctcagctcctggtctatgctgcaacaaacttagcagatggtgtgccatcaaggttcagtggcagtggatcaggcacacagtattccctcaagatcaacagcctgcagtctgaagattttgggaattattactgtcaacatttttggggtactccgtacacgttcggaggggggaccaagctggaaataaaa

SEQ ID NO: 24 - protein, 5 aa, synthetic construct
  REGION 1..5: note = Synthetic
DYIMS

SEQ ID NO: 25 - protein, 17 aa, synthetic construct
  REGION 1..17: note = Synthetic
TVSSGGDNTYYPDSVKG

SEQ ID NO: 26 - protein, 9 aa, synthetic construct
  REGION 1..9: note = Synthetic
YGGLGAMDY

SEQ ID NO: 27 - protein, 11 aa, synthetic construct
  REGION 1..11: note = Synthetic
RASGNIHNYLT

SEQ ID NO: 28 - protein, 7 aa, synthetic construct
  REGION 1..7: note = Synthetic
NAKTLAD

SEQ ID NO: 29 - protein, 9 aa, synthetic construct
  REGION 1..9: note = Synthetic
QHFWSVPYT

SEQ ID NO: 30 - protein, 118 aa, synthetic construct
  REGION 1..118: note = Synthetic
EVQLVESGGGLVKPGGSLKLSCAASGFTFNDYIMSWVRQTPEKRLEWVATVSSGGDNTYYPDSVKGRFTISRDNAKNNLYLQMSSLRSEDTALYYCTRYGGLGAMDYWGQGTSVTVSS

SEQ ID NO: 31 - protein, 107 aa, synthetic construct
  REGION 1..107: note = Synthetic
DIVMTQSPASLSASVGETVTITCRASGNIHNYLTWYQQKQGKSPQLLVYNAKTLADGVPSRFSGSGSGPQYSLKINSLQPEDFGSYYCQHFWSVPYTFGGGTRLEIK

SEQ ID NO: 32 - DNA, 350 bp, synthetic construct
  misc_feature 1..350: note = Synthetic
  source 1..350: mol_type = other DNA
gaagtgcagctggtggagtctgggggaggcttagtgaagcctggagggtccctgaaactctcctgtgcagcctctggattcactttcaatgactatatcatgtcttgggttcgccagactccggagaagaggctggagtgggtcgcaaccgttagtagtggtggtgataacacctactatccagatagtgtgaagggtcgattcaccatctccagagacaatgccaagaacaacctgtacctgcaaatgagcagtctgaggtctgaggacacggccttgtattactgtacaagatatggggggttaggggctatggactactggggtcaaggaacctcagtcaccgtctc

SEQ ID NO: 33 - DNA, 321 bp, synthetic construct
  misc_feature 1..321: note = Synthetic
  source 1..321: mol_type = other DNA
gacattgtgatgacccagtctccagcctccctatctgcatctgtgggagaaactgtcaccatcacatgtcgagcaagtgggaatattcacaattatttaacatggtatcagcagaaacagggaaaatctcctcagctcctggtctataatgcaaaaaccttagcagatggtgtgccatcaaggttcagtggcagtggatcaggaccacaatattctctcaagatcaacagcctgcagcctgaagattttgggagttattactgtcaacatttttggagtgttccgtacacgttcggaggggggaccagactggaaataaaa

SEQ ID NO: 34-60 - skipped (27 entries)

SEQ ID NO: 61 - protein, 118 aa, synthetic construct
  REGION 1..118: note = Synthetic
EVQLLESGGGLVQPGGSLRLSCAASGFTFNDYIMSWVRQAPGKGLEWVATVSSGGDNTYYPDSVKGRFTISRDNSKNTLYLQMNSLRAEDTAVYYCTRYGGLGAMDYWGQGTLVTVSS

SEQ ID NO: 62 - protein, 107 aa, synthetic construct
  REGION 1..107: note = Synthetic
DIQMTQSPSSLSASVGERVTITCRASGNIHNYLTWYQQKPGKSPKLLVYNAKTLADGVPSRFSGSGSGPDYTLTISSLQPEDFATYYCQHFWSVPYTFGQGTKLEIK

SEQ ID NO: 63 - DNA, 354 bp, synthetic construct
  misc_feature 1..354: note = Synthetic
  source 1..354: mol_type = other DNA
gaagtgcagctgctcgagagcgggggcggcctcgtgcaacctggcgggagcctgagactgagctgcgccgctagcggcttcaccttcaacgactacatcatgagctgggtgagacaagcccccggcaagggcctggagtgggtggccaccgtgagcagcgggggcgacaacacctactaccccgacagcgtgaagggcagattcaccatcagcagagacaacagcaagaacaccctgtacctgcagatgaacagcctgagagccgaggacaccgccgtgtactactgcacaagatacggcggcctgggcgccatggactactggggccaaggcaccctggtgaccgtcagctcc

SEQ ID NO: 64 - DNA, 321 bp, synthetic construct
  misc_feature 1..321: note = Synthetic
  source 1..321: mol_type = other DNA
gacattcagatgacacagagccctagcagcctgagcgctagcgtgggcgagagagtgaccatcacctgcagagctagcggcaacatccacaactacctgacctggtatcagcagaagcccggcaagagccccaagctgctggtgtacaacgccaagaccctggccgacggcgtgcctagcagattcagcggcagcggcagcggccccgactacaccctgaccatcagcagcctgcagcccgaggacttcgccacctactactgtcagcacttctggagcgtgccctacaccttcggccaaggcaccaagctggagatcaag

SEQ ID NO: 65 - protein, 7 aa, synthetic construct
  REGION 1..7: note = Synthetic
NAKTLAS

SEQ ID NO: 66 - protein, 107 aa, synthetic construct
  REGION 1..107: note = Synthetic
DIQMTQSPSSLSASVGDRVTITCRASGNIHNYLTWYQQKPGKAPKLLIYNAKTLASGVPSRFSGSGSGTDFTLTISSLQPEDFATYYCQHFWSVPYTFGQGTKLEIK

SEQ ID NO: 67 - DNA, 321 bp, synthetic construct
  misc_feature 1..321: note = Synthetic
  source 1..321: mol_type = other DNA
gacattcagatgacacagagccctagcagcctgagcgctagcgtgggcgacagagtgaccatcacctgcagagctagcggcaacatccacaactacctgacctggtatcagcagaagcccggcaaggcccccaagctgctgatctacaacgccaagaccctggccagcggcgtgcctagcagattcagcggcagcggcagcggcaccgacttcaccctgaccatcagcagcctgcagcccgaggacttcgccacctactactgtcagcacttctggagcgtgccctacaccttcggccaaggcaccaagctggagatcaag

SEQ ID NO: 68 - protein, 107 aa, synthetic construct
  REGION 1..107: note = Synthetic
DIQMTQSPSSLSASVGDRVTITCRASGNIHNYLTWYQQKPGKAPKLLIYNAKTLASGVPSRFSGSGSGTDYTLTISSLQPEDFATYYCQHFWSVPYTFGQGTKLEIK

SEQ ID NO: 69 - DNA, 321 bp, synthetic construct
  misc_feature 1..321: note = Synthetic
  source 1..321: mol_type = other DNA
gacattcagatgacacagagccctagcagcctgagcgctagcgtgggcgacagagtgaccatcacctgcagagctagcggcaacatccacaactacctgacctggtatcagcagaagcccggcaaggcccccaagctgctgatctacaacgccaagaccctggccagcggcgtgcctagcagattcagcggcagcggcagcggcaccgactacaccctgaccatcagcagcctgcagcccgaggacttcgccacctactactgtcagcacttctggagcgtgccctacaccttcggccaaggcaccaagctggagatcaag

SEQ ID NO: 70 - protein, 20 aa, synthetic construct
  REGION 1..20: note = Synthetic
GGGGSGGGGSGGGGSGGGGS

SEQ ID NO: 71 - protein, 15 aa, synthetic construct
  REGION 1..15: note = Synthetic
GGGGSGGGGSGGGGS

SEQ ID NO: 72 - protein, 10 aa, synthetic construct
  REGION 1..10: note = Synthetic
GGGGSGGGGS

SEQ ID NO: 73-100 - skipped (28 entries)

SEQ ID NO: 101 - protein, 254 aa, synthetic construct
  REGION 1..254: note = Synthetic
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

SEQ ID NO: 102 - protein, 254 aa, synthetic construct
  REGION 1..254: note = Synthetic
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 103 - protein, 233 aa, synthetic construct
  REGION 1..233: note = Synthetic
MWQLLLPTALLLLVSAGMRTEDLPKAVVFLEPQWYRVLEKDSVTLKCQGAYSPEDNSTQWFHNENLISSQASSYFIDAATVDDSGEYRCQTNLSTLSDPVQLEVHVGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 104 - protein, 233 aa, synthetic construct
  REGION 1..233: note = Synthetic
MWQLLLPTALLLLVSAGMRTEDLPKAVVFLEPQWYSVLEKDSVTLKCQGAYSPEDNSTQWFHNESLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 105 - protein, 233 aa, synthetic construct
  REGION 1..233: note = Synthetic
MWQLLLPTALLLLVSAGMRTEDLPKAVVFLEPQWYSVLEKDSVTLKCQGAYSPEDNSTQWFHNENLISSQASSYFIDAATVNDSGEYRCQTNLSTLSDPVQLEVHIGWLLLQAPRWVFKEEDPIHLRCHSWKNTALHKVTYLQNGKDRKYFHHNSDFHIPKATLKDSGSYFCRGLVGSKNVSSETVNITITQGLAVSTISSFSPPGYQVSFCLVMVLLFAVDTGLYFSVKTNI

SEQ ID NO: 106 - protein, 254 aa, synthetic construct
  REGION 1..254: note = Synthetic
MWQLLLPTALLLLVSAGMRAEDLPKAVVFLEPQWYRVLEKDRVTLKCQGAYSPEDNSTRWFHNESLISSQTSSYFIAAARVNNSGEYRCQTSLSTLSDPVQLEVHIGWLLLQAPRWVFKEEESIHLRCHSWKNTLLHKVTYLQNGKGRKYFHQNSDFYIPKATLKDSGSYFCRGLIGSKNVSSETVNITITQDLAVSSISSFFPPGYQVSFCLVMVLLFAVDTGLYFSMKKSIPSSTRDWEDHKFKWSKDPQDK

SEQ ID NO: 107 - protein, 231 aa, synthetic construct
  REGION 1..231: note = Synthetic
EPKSSDKTHTCPPCPAPPAAGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 241 aa, synthetic construct
  REGION 1..241: note = Synthetic
EVQLVQSGAEVKKPGESLKVSCKASGYTFTSYYMHWVRQAPGQGLEWMGIINPSGGSTSYAQKFQGRVTMTRDTSTSTVYMELSSLRSEDTAVYYCARGSAYYYDFADYWGQGTLVTVSSGGGGSGGGGSGGGGSQPVLTQPSSVSVAPGQTATISCGGHNIGSKNVHWYQQRPGQSPVLVIYQDNKRPSGIPERFSGSNSGNTATLTISGTQAMDEADYYCQVWDNYSVLFGGGTKLTVL

SEQ ID NO: 109 - protein, 5 aa, synthetic construct
  REGION 1..5: note = Synthetic
SYAVG

SEQ ID NO: 110 - protein, 17 aa, synthetic construct
  REGION 1..17: note = Synthetic
AIRRSDGTTDYVDSVKG

SEQ ID NO: 111 - protein, 11 aa, synthetic construct
  REGION 1..11: note = Synthetic
SDSDYEPLGDY

SEQ ID NO: 112 - protein, 120 aa, synthetic construct
  REGION 1..120: note = Synthetic
QVQLVESGGGVVQPGGSLTLSCEASGRTFGSYAVGWFRQALGRGREFVAAIRRSDGTTDYVDSVKGRFTISRDNAKNTVYLQMNSLKPEDTAVYYCAASDSDYEPLGDYWGQGTQVTVSS

SEQ ID NO: 113 - DNA, 360 bp, synthetic construct
  misc_feature 1..360: note = Synthetic
  source 1..360: mol_type = other DNA
caggtgcagctcgtggagtctgggggcggcgtggtgcagcctggggggtctctgacactctcctgtgaagcctctggacgcaccttcggcagctatgccgtgggctggttccgccaggctcttgggagggggcgtgagtttgtagcagctattaggcggagtgatggtactacagactatgtagactccgtgaagggccgattcaccatctccagagacaacgccaagaacacggtgtatctgcaaatgaacagcctgaaacctgaggacacggccgtttattactgtgcagcatcggatagcgactatgaacccttaggggactactggggccaggggacccaggtcaccgtctcctca

SEQ ID NO: 114 - protein, 17 aa, synthetic construct
  REGION 1..17: note = Synthetic
AIRRSDATTDYVDSVKG

SEQ ID NO: 115 - protein, 120 aa, synthetic construct
  REGION 1..120: note = Synthetic
EVQLVESGGGLVQPGGSLRLSCAASGRTFGSYAVGWFRQAPGKGREWVAAIRRSDATTDYVDSVKGRFTISRDNAKNSVYLQMNSLRPEDTAVYYCAASDSDYEPLGDYWGQGTLVTVSS

SEQ ID NO: 116 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcaagggcagagagtgggtggccgccatcagaagaagcgacgccaccaccgactacgtggacagcgtgaagggcagattcaccatcagcagagacaacgccaagaacagcgtgtacctgcagatgaacagcctgagacccgaggacaccgccgtgtactactgcgccgcctccgacagcgactacgagcctctgggcgattactggggccaaggcaccctggtgaccgtgagcagc

SEQ ID NO: 117 - protein, 120 aa, synthetic construct
  REGION 1..120: note = Synthetic
EVQLVESGGGLVQPGGSLRLSCAASGRTFGSYAVGWFRQAPGRGREFVAAIRRSDGTTDYVDSVKGRFTISRDNAKNTVYLQMNSLKPEDTAVYYCAASDSDYEPLGDYWGQGTLVTVSS

SEQ ID NO: 118 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcagaggcagagagttcgtggccgccatcagaagaagcgacggcaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagc

SEQ ID NO: 119 - protein, 120 aa, synthetic construct
  REGION 1..120: note = Synthetic
EVQLVESGGGLVQPGGSLRLSCAASGRTFGSYAVGWFRQAPGKGREFVAAIRRSDATTDYVDSVKGRFTISRDNAKNTVYLQMNSLKPEDTAVYYCAASDSDYEPLGDYWGQGTLVTVSS

SEQ ID NO: 120 - DNA, 360 bp, synthetic construct
  misc_feature 1..360: note = Synthetic
  source 1..360: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcaagggcagagagttcgtggccgccatcagaagaagcgacgccaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagc

SEQ ID NO: 121 - protein, 449 aa, synthetic construct
  REGION 1..449: note = Synthetic
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 216 aa, synthetic construct
  REGION 1..216: note = Synthetic
DIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123-142 - skipped (20 entries)

SEQ ID NO: 143 - protein, 482 aa, synthetic construct
  REGION 1..482: note = Synthetic
EVQLVESGGGLVQPGGSLRLSCAASGRTFGSYAVGWFRQAPGKGREFVAAIRRSDATTDYVDSVKGRFTISRDNAKNTVYLQMNSLKPEDTAVYYCAASDSDYEPLGDYWGQGTLVTVSSGGGGSGGGGSEVQLVESGGGLVQPGGSLRLSCAASGRTFGSYAVGWFRQAPGKGREFVAAIRRSDATTDYVDSVKGRFTISRDNAKNTVYLQMNSLKPEDTAVYYCAASDSDYEPLGDYWGQGTLVTVSSEPKSSDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - DNA, 1449 bp, synthetic construct
  misc_feature 1..1449: note = Synthetic
  source 1..1449: mol_type = other DNA
gaggtgcagctcgtggaatccgggggcgggctggtgcaacccggcgggagcctgagactgagctgcgccgctagcggcagaaccttcggcagctacgccgtgggctggttcagacaagcccccggcaagggcagagagttcgtggccgccatcagaagaagcgacgccaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagcggcggtggcggctctggtggcggcggctctgaggtgcagctcgtggaatccgggggcgggctggtgcaacccggcgggagcctgagactgagctgcgccgctagcggcagaaccttcggcagctacgccgtgggctggttcagacaagcccccggcaagggcagagagttcgtggccgccatcagaagaagcgacgccaccaccgactacgtggacagcgtgaagggcagattcaccatcagcagagacaacgccaagaacaccgtgtacctgcagatgaacagcctgaagcccgaggacaccgccgtgtactactgcgccgcctccgacagcgactacgagcctctgggcgattactggggccaaggcaccctggtgaccgtgagcagcgagccaaagtcctctgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 145 - protein, 448 aa, synthetic construct
  REGION 1..448: note = Synthetic
EVQLLESGGGLVQPGGSLRLSCAASGFTFNDYIMSWVRQAPGKGLEWVATVSSGGDNTYYPDSVKGRFTISRDNSKNTLYLQMNSLRAEDTAVYYCTRYGGLGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 146 - DNA, 1347 bp, synthetic construct
  misc_feature 1..1347: note = Synthetic
  source 1..1347: mol_type = other DNA
gaagtgcagctgctcgagagcgggggcggcctcgtgcaacctggcgggagcctgagactgagctgcgccgctagcggcttcaccttcaacgactacatcatgagctgggtgagacaagcccccggcaagggcctggagtgggtggccaccgtgagcagcgggggcgacaacacctactaccccgacagcgtgaagggcagattcaccatcagcagagacaacagcaagaacaccctgtacctgcagatgaacagcctgagagccgaggacaccgccgtgtactactgcacaagatacggcggcctgggcgccatggactactggggccaaggcaccctggtgaccgtcagctccgctagcaccaaggggccctcggtcttccccctggcaccctcctccaagagtacttctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctctacatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgtcctgcgccgtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacccagaagagcctctccctgtctccgggtaaatga

SEQ ID NO: 147 - protein, 214 aa, synthetic construct
  REGION 1..214: note = Synthetic
DIQMTQSPSSLSASVGDRVTITCRASGNIHNYLTWYQQKPGKAPKLLIYNAKTLASGVPSRFSGSGSGTDFTLTISSLQPEDFATYYCQHFWSVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - DNA, 645 bp, synthetic construct
  misc_feature 1..645: note = Synthetic
  source 1..645: mol_type = other DNA
gacattcagatgacacagagccctagcagcctgagcgctagcgtgggcgacagagtgaccatcacctgcagagctagcggcaacatccacaactacctgacctggtatcagcagaagcccggcaaggcccccaagctgctgatctacaacgccaagaccctggccagcggcgtgcctagcagattcagcggcagcggcagcggcaccgacttcaccctgaccatcagcagcctgcagcccgaggacttcgccacctactactgtcagcacttctggagcgtgccctacaccttcggccaaggcaccaagctggagatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttga

SEQ ID NO: 149 - protein, 4 aa, synthetic construct
  REGION 1..4: note = Synthetic
GGGS

SEQ ID NO: 150 - protein, 4 aa, synthetic construct
  REGION 1..4: note = Synthetic
SGGG

SEQ ID NO: 151 - protein, 5 aa, synthetic construct
  REGION 1..5: note = Synthetic
GGGGS

SEQ ID NO: 152 - protein, 7 aa, synthetic construct
  REGION 1..7: note = Synthetic
GGGGSGS

SEQ ID NO: 153 - protein, 7 aa, synthetic construct
  REGION 1..7: note = Synthetic
GGGGSGS

SEQ ID NO: 154 - protein, 8 aa, synthetic construct
  REGION 1..8: note = Synthetic
GGGGSGGS

SEQ ID NO: 155 - protein, 10 aa, synthetic construct
  REGION 1..10: note = Synthetic
GGGGSGGGGS

SEQ ID NO: 156 - protein, 15 aa, synthetic construct
  REGION 1..15: note = Synthetic
GGGGSGGGGSGGGGS

SEQ ID NO: 157 - protein, 16 aa, synthetic construct
  REGION 1..16: note = Synthetic
AKTTPKLEEGEFSEAR

SEQ ID NO: 158 - protein, 17 aa, synthetic construct
  REGION 1..17: note = Synthetic
AKTTPKLEEGEFSEARV

SEQ ID NO: 159 - protein, 9 aa, synthetic construct
  REGION 1..9: note = Synthetic
AKTTPKLGG

SEQ ID NO: 160 - protein, 10 aa, synthetic construct
  REGION 1..10: note = Synthetic
SAKTTPKLGG

SEQ ID NO: 161 - protein, 17 aa, synthetic construct
  REGION 1..17: note = Synthetic
AKTTPKLEEGEFSEARV

SEQ ID NO: 162 - protein, 6 aa, synthetic construct
  REGION 1..6: note = Synthetic
SAKTTP

SEQ ID NO: 163 - protein, 10 aa, synthetic construct
  REGION 1..10: note = Synthetic
SAKTTPKLGG

SEQ ID NO: 164 - protein, 6 aa, synthetic construct
  REGION 1..6: note = Synthetic
RADAAP

SEQ ID NO: 165 - protein, 9 aa, synthetic construct
  REGION 1..9: note = Synthetic
RADAAPTVS

SEQ ID NO: 166 - protein, 12 aa, synthetic construct
  REGION 1..12: note = Synthetic
RADAAAAGGPGS

SEQ ID NO: 167 - protein, 27 aa, synthetic construct
  REGION 1..27: note = Synthetic
RADAAAAGGGGSGGGGSGGGGSGGGGS

SEQ ID NO: 168 - protein, 6 aa, synthetic construct
  REGION 1..6: note = Synthetic
SAKTTP

SEQ ID NO: 169 - protein, 10 aa, synthetic construct
  REGION 1..10: note = Synthetic
SAKTTPKLGG

SEQ ID NO: 170 - protein, 18 aa, synthetic construct
  REGION 1..18: note = Synthetic
SAKTTPKLEEGEFSEARV

SEQ ID NO: 171 - protein, 5 aa, synthetic construct
  REGION 1..5: note = Synthetic
ADAAP

SEQ ID NO: 172 - protein, 12 aa, synthetic construct
  REGION 1..12: note = Synthetic
ADAAPTVSIFPP

SEQ ID NO: 173 - protein, 5 aa, synthetic construct
  REGION 1..5: note = Synthetic
TVAAP

SEQ ID NO: 174 - protein, 12 aa, synthetic construct
  REGION 1..12: note = Synthetic
TVAAPSVFIFPP

SEQ ID NO: 175 - protein, 6 aa, synthetic construct
  REGION 1..6: note = Synthetic
QPKAAP

SEQ ID NO: 176 - protein, 13 aa, synthetic construct
  REGION 1..13: note = Synthetic
QPKAAPSVTLFPP

SEQ ID NO: 177 - protein, 6 aa, synthetic construct
  REGION 1..6: note = Synthetic
AKTTPP

SEQ ID NO: 178 - protein, 13 aa, synthetic construct
  REGION 1..13: note = Synthetic
AKTTPPSVTPLAP

SEQ ID NO: 179 - protein, 6 aa, synthetic construct
  REGION 1..6: note = Synthetic
AKTTAP

SEQ ID NO: 180 - protein, 13 aa, synthetic construct
  REGION 1..13: note = Synthetic
AKTTAPSVYPLAP

SEQ ID NO: 181 - protein, 6 aa, synthetic construct
  REGION 1..6: note = Synthetic
ASTKGP

SEQ ID NO: 182 - protein, 13 aa, synthetic construct
  REGION 1..13: note = Synthetic
ASTKGPSVFPLAP

SEQ ID NO: 183 - protein, 15 aa, synthetic construct
  REGION 1..15: note = Synthetic
GENKVEYAPALMALS

SEQ ID NO: 184 - protein, 15 aa, synthetic construct
  REGION 1..15: note = Synthetic
GPAKELTPLKEAKVS

SEQ ID NO: 185 - protein, 15 aa, synthetic construct
  REGION 1..15: note = Synthetic
GHEAAAVMQVQYPAS

SEQ ID NO: 186 - protein, 117 aa, synthetic construct
  REGION 1..117: note = Synthetic
EVQLVESGGGLVQPGGSMRLSCVASGFPFSNYWMNWVRQAPGKGLEWVGEIRLKSNNYTTHYAESVKGRFTISRDDSKNSLYLQMNSLKTEDTAVYYCTRHYYFDYWGQGTLVTVSS

SEQ ID NO: 187 - protein, 112 aa, synthetic construct
  REGION 1..112: note = Synthetic
DIVMTQSPLSNPVTPGEPASISCRSSKSLLHSNGITYFFWYLQKPGQSPQLLIYQMSNLASGVPDRFSGSGSGTDFTLRISRVEAEDVGVYYCAQNLELPPTFGQGTKVEIK

SEQ ID NO: 188 - protein, 119 aa, synthetic construct
  REGION 1..119: note = Synthetic
QVQLQQSGAEVKKPGASVKVSCEASGYTFPSYVLHWVKQAPGQGLEWIGYINPYNDGTQYNEKFKGKATLTRDTSINTAYMELSRLRSDDTAVYYCARGFGGSYGFAYWGQGTLVTVSS

SEQ ID NO: 189 - protein, 108 aa, synthetic construct
  REGION 1..108: note = Synthetic
DIQLTQSPSSLSASVGDRVTMTCSASSSVSSSYLYWYQQKPGKAPKLWIYSTSNLASGVPARFSGSGSGTDFTLTISSLQPEDSASYFCHQWNRYPYTFGGGTRLEIK

SEQ ID NO: 190 - protein, 118 aa, synthetic construct
  REGION 1..118: note = Synthetic
QVQLVQSGAEVKKPGASVKVSCKASGYTFSAYWIEWVRQAPGKGLEWVGEILPGSNNSRYNEKFKGRVTVTRDTSTNTAYMELSSLRSEDTAVYYCARSYDFAWFAYWGQGTLVTVSS

SEQ ID NO: 191 - protein, 113 aa, synthetic construct
  REGION 1..113: note = Synthetic
DIQMTQSPSSLSASVGDRVTITCKSSQSLLYSSNQKIYLAWYQQKPGKAPKLLIYWASTRESGVPSRFSGSGSGTDFTFTISSLQPEDIATYYCQQYYRYPRTFGQGTKVEIK

SEQ ID NO: 192 - protein, 122 aa, synthetic construct
  REGION 1..122: note = Synthetic
DVQVQESGPDLVKPSQSLSLTCTVTGHSITRGSSWHWIRQFPGNKLEWMGYIHYGGGTSYNPSLKSRISITRDTSKNQFFLQLNSVTTEDTATFFCARYSYDITYRWFFDVWGAGTTVIVSS

SEQ ID NO: 193 - protein, 107 aa, synthetic construct
  REGION 1..107: note = Synthetic
DILLTQSPAILSVSPGERVSFSCRASQNIGTSIHWYQQRKNGSPRLLIKYASESISGIPSRFSGSGSGTDFTLSINSVESEDMADYYCQQNNNWPLTFGAGTKLELK

SEQ ID NO: 194 - protein, 119 aa, synthetic construct
  REGION 1..119: note = Synthetic
EVQLVQSGAEVKKPGESLKISCKGSGYAFSNFWMNWVRQMPGKGLEWMGQIYPGDGDTNYNGKFKGQVTISADKSISTAYLQWSSLKASDTAMYYCARSYYRSAWFAYWGQGTLVTVSL

SEQ ID NO: 195 - protein, 107 aa, synthetic construct
  REGION 1..107: note = Synthetic
EIVLTQSPDFQSVTPKEKVTITCRASQSIGTSIHWYQQKPDQSPKLLIKYASESISGVPSRFSGSGSGTDFTLTINSLEAEDAATYYCQQSNNWPLTFGQGTKVEIK